(19) United States
(12) Patent Application Publication
(10) Pub. No.: US 2009/0029981 A1
(43) Pub. Date: Jan. 29, 2009

(54) SUBSTITUTED 4-PHENYLPIPERIDINES

(76) Inventors: Peter Herold, Allschwil (CH); Robert Mah, Allschwil (CH); Vincenzo Tschinke, Allschwil (CH); Dirk Behnke, Allschwil (CH); Nathalie Jotterand, Allschwil (CH); Aleksandar Stojanovic, Allschwil (CH); Stefan Stutz, Allschwil (CH); Stjepan Jelakovic, Allschwil (CH); Michael Quirmbach, Basel (CH)

Correspondence Address: WENDEROTH, LIND & PONACK, L.L.P., 2033 K STREET N. W., SUITE 800, WASHINGTON, DC, 20006-1021, US

(21) Appl. No.: 12/087,904

(22) PCT Filed: Jan. 18, 2007
(86) PCT No.: PCT/EP2007/050481

§ 371 Date: Jul. 17, 2008

(30) Foreign Application Priority Data
Jan. 19, 2006 (CH) 87/06

(51) Int. Cl. A61K 31/538; A61P 9/00; C07D 413/12; C07D 413/14

(52) U.S. Cl. 514/230.5; 544/105; 544/73

(57) ABSTRACT

The application relates to substituted 4-phenylpiperidines of the general formula and their salts, preferably their pharmaceutically acceptable salts, in which R², R³, W and X have the meanings explained in the description, a process for their preparation and the use of these compounds as medicines, especially as renin inhibitors.

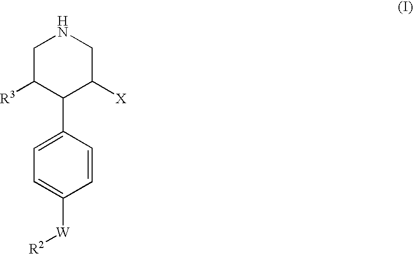

### FIELD OF THE INVENTION

The present invention relates to novel substituted 4-phenylpiperidines, process for their preparation and the use of the compounds as medicines, especially as renin inhibitors.

### BACKGROUND OF THE INVENTION

Piperidine derivatives for use as medicines are disclosed for example in WO97/09311. However, in terms in particular of the renin inhibition, there continues to be a need for highly potent active ingredients. The priority in this connection is improving the pharmacokinetic properties. These properties, which are directed at better bioavailability, are for example absorption, metabolic stability, solubility or lipophilicity.

### DETAILED DESCRIPTION OF THE INVENTION

The invention therefore relates firstly to substituted 4-phenylpiperidines of the general formula

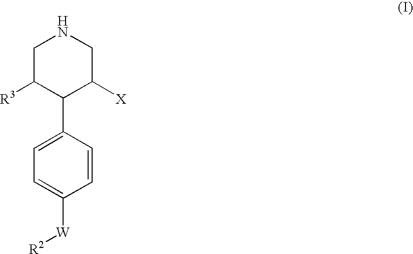

in which
(A) R¹ is heterocyclyl substituted by oxo or oxide or as indicated under (B) or (C), in particular azepanyl, benzo[1,3]dioxolyl, benzofuranyl, benzoimidazolyl, 4H-benzo[1,4]oxazinyl, benzooxazolyl, 4H-benzo[1,4]thiazinyl, quinolinyl, chromenyl, dihydro-benzo[e][1,4]diazepinyl, dihydrobenzofuranyl, 3,4-dihydro-2H-benzo[1,4]oxazinyl, dihydro-3H-benzo[1,4]oxazinyl, dihydrobenzo[d][1,3]oxazinyl, 3,4-dihydro-2H-benzo[1,4]thiazinyl, dihydro-2H-1λ6-benzo[1,4]thiazinyl, dihydro-1H-quinazolinyl, 1a,7b-dihydro-1H-cyclopropa[c]chromenyl, dihydroimidazolyl, 1,3-dihydroindolyl, 2,3-dihydroindolyl, dihydro-1H-pyrido[2,3-b][1,4]oxazinyl, indazolyl, indolyl, 3H-isobenzofuranyl, [1,5]naphthyridyl, oxazolyl, phthalazinyl, piperidinyl, pyrazolyl, 1H-pyrido[2,3-b][1,4]oxazinyl, pyridyl, 1H-pyrrolizinyl, 1H-pyrrolo[2,3-b]pyridyl, pyrrolyl, tetrahydrobenzo[e][1,4]diazepinyl, 3H-thieno[2,3-d]pyrimidinyl, tetrahydro-quinoxalinyl, 1,1a,2,7b-tetrahydrocyclopropa[c]chromenyl, tetrahydropyranyl or triazinyl; or
(B) R¹ is aryl which is substituted by 1-4-acetamidinyl-C₁₋₈alkyl, acyl-C₁₋₈alkoxy-C₁₋₈alkyl, (N-acyl)-C₁₋₈alkoxy-C₁₋₈alkylamino, C₁₋₈alkoxy, C₁₋₈alkoxy-C₁₋₈alkoxy, C₁₋₈alkoxy-C₁₋₈alkoxy-C₁₋₈alkyl, C₁₋₈alkoxy-C₁₋₈alkyl, (N—C₁₋₈alkoxy)-C₁₋₈alkylaminocarbonyl-C₁₋₈alkoxy, (N—C₁₋₈alkoxy)-C₁₋₈alkylaminocarbonyl-C₁₋₈alkyl, C₁₋₈alkoxy-C₁₋₈alkyl-carbamoyl, C₁₋₈alkoxy-C₁₋₈alkylcarbonyl, C₁₋₈alkoxy-C₁₋₈alkylcarbonylamino, 1-C₁₋₈alkoxy-C₁₋₈alkylimidazol-2-yl, 2-C₁₋₈alkoxy-C₁₋₈alkyl-4-oxoimidazol-1-yl, 1-C₁₋₈alkoxy-C₁₋₈-alkyltetrazol-5-yl, 5-C₁₋₈alkoxy-C₁₋₈-alkyltetrazol-1-yl, 6-alkoxy-aminocarbonyl-C₁₋₈alkoxy, C₁₋₈alkoxyaminocarbonyl-C₁₋₈alkyl, C₁₋₈alkoxycarbonyl, C₁₋₈alkoxycarbonyl-C₁₋₈alkoxy, C₁₋₈alkoxycarbonyl-C₁₋₈alkyl, C₁₋₈alkoxycarbonylamino-C₁₋₈alkoxy, C₁₋₈alkoxycarbonylamino-C₁₋₈alkyl, C₁₋₈alkyl, (N—C₁₋₈alkyl)-C₁₋₈alkoxy-C₁₋₈alkylcarbamoyl, (N—C₁₋₈alkyl)-C₁₋₈alkoxy-C₁₋₈alkylcarbonylamino, (N—C₁₋₈alkyl)-C₁₋₈alkoxycarbonylamino, (N—C₁₋₈alkyl)-C₀₋₈alkylcarbonylamino-C₁₋₈alkoxy, (N—C₁₋₈alkyl)-C₀₋₈alkylcarbonylamino-C₁₋₈alkyl, (N—C₁₋₈alkyl)-C₁₋₈alkylsulfonylamino-C₁₋₈alkoxy, (N—C₁₋₈alkyl)-C₁₋₈alkylsulfonylamino-C₁₋₈alkyl, C₁₋₈alkylamidinyl, C₁₋₈alkyl-aminocarbonyl-C₁₋₈alkoxy, di-C₁₋₈alkylaminocarbonyl-C₁₋₈alkoxy, C₁₋₈alkylamino-carbonyl-C₁₋₈alkoxy-C₁₋₈alkyl, C₁₋₈alkylaminocarbonyl-C₁₋₈alkyl, C₁₋₈alkylamino-carbonylamino-C₁₋₈alkoxy, C₁₋₈alkylaminocarbonylamino-C₁₋₈alkyl, di-C₁₋₈alkylamino-carbonyl-C₁₋₈alkyl, C₁₋₈alkylamino-C₂₋₈alkoxy, di-C₁₋₈alkylamino-C₂₋₈alkoxy, C₁₋₈alkyl-amino-C₁₋₈alkyl, di-C₁₋₈alkylamino-C₁₋₈alkyl, C₁₋₈alkylcarbamoyl, di-C₁₋₈alkylcarbamoyl, C₀₋₈alkylcarbonylamino-C₁₋₈alkoxy, C₀₋₈alkylcarbonylamino, C₀₋₈alkylcarbonylamino-C₁₋₈alkyl, C₁₋₈alkylcarbonyloxy-C₁₋₈alkoxy, C₁₋₈alkylcarbonyloxy-C₁₋₈alkyl, C₁₋₈alkyl-sulfonyl, C₁₋₈alkylsulfonyl-C₁₋₈alkoxy, C₁₋₈alkylsulfonyl-C₁₋₈alkyl, C₁₋₈alkylsulfonylamino-C₁₋₈alkoxy, C₁₋₈alkylsulfonylamino-C₁₋₈alkyl, carbamoyl, carbamoyl-C₁₋₈alkoxy, carbamoyl-C₁₋₈alkyl, carboxy-C₁₋₈alkoxy, carboxy-C₁₋₈alkoxy-C₁₋₈alkyl, carboxy-C₁₋₈alkyl, cyano, cyano-C₁₋₈alkoxy, cyano-C₁₋₈alkyl, C₃₋₈cycloalkyl-C₁₋₈alkoxy, C₃₋₈cycloalkyl-C₁₋₈alkyl, C₃₋₈cycloalkylcarbonylamino-C₁₋₈alkoxy, C₃₋₈cycloalkyl-carbonylamino-C₁₋₈alkyl, O,N-dimethylhydroxylamino-C₁₋₈alkyl, halogen, hydroxy-C₁₋₈alkoxy-C₁₋₈alkoxy, hydroxy-C₁₋₈alkoxy-C₁₋₈alkyl, hydroxy-C₁₋₈alkyl, (N-hydroxy)-C₁₋₈alkylaminocarbonyl-C₁₋₈alkoxy, (N-hydroxy)-C₁₋₈alkylaminocarbonyl-C₁₋₈alkyl, (N-hydroxy)aminocarbonyl-C₁₋₈alkoxy, (N-hydroxy)aminocarbonyl-C₁₋₈alkyl, 2-oxooxazolidinyl-C₁₋₈alkoxy, 2-oxooxazolidinyl-C₁₋₈alkyl, O-methyloximyl-C₁₋₈alkyl, polyhalo-C₁₋₈alkoxy or polyhalo-C₁₋₈alkyl; or
(C) R¹ is aryl which is substituted by 3-acetamidomethylpyrrolidinyl, 3-C₁₋₈alkoxy-C₁₋₈alkyl-pyrrolidinyl, 3,4-dihydroxypyrrolidinyl, 2,6-dimethylmorpholinyl, 3,5-dimethyl-morpholinyl, dioxanyl, dioxolanyl, 4,4-dioxothiomorpholinyl, dithianyl, dithiolanyl, 2-hydroxymethylpyrrolidinyl, 4-hydroxypiperidinyl, 3-hydroxypyrrolidinyl, imidazolyl-alkoxy, imidazolylalkyl, 2-methylimidazolylalkoxy, 2-methylimidazolylalkyl, 3-methyl-[1,2,4]-oxadiazol-5-ylalkoxy, 5-methyl-[1,2,4]-oxadiazol-3-ylalkoxy, 3-methyl-[1,2,4]-oxadiazol-5-ylalkyl, 5-methyl-[1,2,4]-oxadiazol-3-ylalkyl, 4-methylpiperazinyl, 5-methyltetrazol-1-ylalkoxy, 5-methyltetrazol-1-ylalkyl, morpholinyl, [1,2,4]-oxadiazol-5-ylalkoxy, [1,2,4]-oxadiazol-5-ylalkyl, oxazol-4-ylalkoxy, oxazol-4-ylalkyl, 2-oxo-[1,3]oxazinyl, 2-oxooxazolidinyl, 2-oxoimidazolidinyl, 2-oxopyrrolidinyl, 4-oxo-piperidinyl, 2-oxopyrrolidinylalkoxy, 2-oxopyrrolidinylalkyl, 2-oxotetrahydropyrimidinyl 4-oxothiomorpholinyl, piperazinyl, piperidinyl, pyrrolidinyl, pyrrolyl, [1,2,4]-triazol-1-yl-alkoxy, [1,2,4]-triazol-4-ylalkoxy, [1,2,4]-triazol-1-ylalkyl, [1,2,4]-triazol-4-ylalkyl, tetrazol-1-ylalkoxy, tetrazol-2-ylalkoxy, tetrazol-5-ylalkoxy, tetrazol-1-ylalkyl, tetrazol-2-ylalkyl, tetrazol-5-ylalkyl, thiazol-4-ylalkoxy, thiazol-4-ylalkyl or thiomorpholinyl; or
(D) R¹ is aryl when X is —O—CHR⁶—CO—NR⁴—R¹ or —O—CHR⁶—CO—NR⁴-Z, where Z is Alk-R¹ where Alk is C₁₋₈alkylene; or
(E) R¹ is aryl when X is —O-Z, where Z is Alk-NR⁴—R¹ or X is −Z, where Z is −Alk-NR⁴—R¹, where Alk is C₁₋₈alkylene;
R² a) is absent when W is cyano; or
   b) is C₁₋₈alkyl, C₂₋₈alkenyl, C₂₋₈alkynyl, C₁₋₈alkoxy-C₁₋₈alkyl, C₁₋₈alkoxy-C₃₋₈cycloalkyl-C₁₋₈-alkyl, C₁₋₈alkylsulfanyl-C₁₋₈alkyl, C₁₋₈alkylsulfonyl-C₁₋₈alkyl when W is —O— or —S—;
R³ a) is halogen- and/or hydroxy-substituted C₁₋₈alkoxy, halogen- and/or hydroxy-substituted C₁₋₈alkoxy-C₁₋₈alkoxy, branched C₁₋₈alkoxy-C₁₋₈alkoxy, optionally N-mono- or N,N-di-C₁₋₈-alkylated amino-C₁₋₈alkoxy, optionally N—C₁₋₈alkylated C₁₋₈alkoxy-C₁₋₈alkylamino-C₁₋₈alkoxy, optionally N-mono- or N,N-di-C₁₋₈alkylated amino-C₀₋₈-alkylcarbonyl-C₁₋₈alkoxy, hydroxy-C₀₋₈-alkylcarbonyl-C₀₋₈alkoxy, C₁₋₈alkoxy-C₀₋₈alkylcarbonyl-C₀₋₈alkoxy, C₁₋₈-alkylcarbonylamino-C₁₋₈alkoxy, cyano-C₁₋₈alkoxy, substituted C₃₋₈cycloalkyl-C₀₋₈alkoxy, heterocyclyl-C₀₋₈alkoxy, optionally N—C₁₋₈alkylated heterocyclyl-C₀₋₈alkylamino-C₀₋₈alkylcarbonyl-C₀₋₈alkoxy, C₁₋₈alkyl-sulfonyl-C₁₋₈alkoxy, C₂₋₈alkynyloxy, heterocyclyl-C₂₋₈alkynyloxy, optionally N-mono- or N,N-di-C₁₋₈alkylated amino-C₂₋₈alkynyloxy, N-mono- or N,N-di-C₁₋₈alkylated aminocarbonyl-C₂₋₈alkynyloxy, heterocyclylcarbonyl-C₀₋₈alkoxy, optionally N-mono- or N,N-di-C₁₋₈alkylated amino-C₁₋₈alkyl, optionally N—C₁₋₈alkylated C₁₋₈alkoxy-C₁₋₈alkylamino-C₁₋₈alkyl, optionally N-mono- or N,N-di-C₁₋₈alkylated and optionally hydroxy-substituted amino-C₀₋₈alkyl-carbonyl-C₀₋₈alkyl, optionally N—C₁₋₈alkylated heterocyclyl-C₀₋₈alkylamino-C₀₋₈alkylcarbonyl-C₀₋₈alkyl, optionally halogen- or hydroxy-substituted C₁₋₈alkoxy-C₁₋₈alkyl, optionally halogen- or hydroxy-substituted hydroxy-C₁₋₈alkyl, optionally N—C₁₋₈alkylated hydroxy-C₁₋₈alkylamino-C₁₋₈alkyl, heterocyclylcarbonyl-C₀₋₈alkyl, heterocyclylcarbonyl-C₀₋₈alkylamino-C₁₋₈alkyl, heterocyclyl-C₁₋₈alkyl, C₁₋₈alkoxycarbonylamino-C₁₋₈alkyl, optionally halogen-substituted heterocyclyl-C₀₋₈alkylcarbonylamino-C₁₋₈alkyl, optionally halogen-substituted C₃₋₈cycloalkyl-C₀₋₈alkylcarbonylamino-C₁₋₈alkyl or optionally halogen-substituted C₁₋₈alkylcarbonylamino-C₁₋₈alkyl; or additionally
   b) is hydroxy, unsubstituted C₁₋₈alkoxy, unsubstituted, unbranched C₁₋₈alkoxy-C₁₋₈-alkoxy or unsubstituted C₃₋₈cycloalkyl-C₀₋₈alkoxy if —W—R² is not C₁₋₈alkoxy;
R⁴ is acyl, C₁₋₈alkoxy-C₁₋₈alkyl, C₁₋₈alkyl, aryl-C₁₋₈alkyl, C₃₋₈cycloalkyl-C₀₋₈alkyl, or hydrogen;
R⁵ is C₁₋₈alkoxycarbonyl-C₁₋₈alkyl, C₁₋₈alkyl, carboxy-C₁₋₈alkyl or hydrogen;
R⁶ is acyl, C₂₋₈alkenyl, C₁₋₈alkyl, aryl-C₁₋₈alkyl or hydrogen;
X is Z, —O-Z or —S-Z, where the bond originating from an oxygen or sulfur atom leads to a saturated C atom of the group Z, or is a group —CHR⁶-Z, —CHOR⁴-Z, —O—CO-Z, —O—CO—R¹, —CO-Z, —C═NOR⁵-Z, —O—CHR⁶-Z, —O—CHR⁶—CO—NR⁴-Z, —O—CHR⁶—CO—NR⁴—R¹, or —O—CHR⁶—R¹;
W is —O—, —S— or cyano;
Z is C₁₋₈-Alk-R¹, C₂₋₈alkenylene-R¹, hydroxy-substituted −Alk-R¹, —O—R¹, —S—R¹, —O-Alk-R¹, —S-Alk-R¹, −Alk-O—R¹, −Alk-S—R¹ or −Alk-NR⁴—R¹, where Alk is C₁₋₈alkylene; and where
   (a) X is —CH—R⁶-Z if Z is —O—R¹ or —S—R¹
   (b) X is —CH—R⁶-Z if Z is —O-Alk-R¹ or —S-Alk-R¹; and
   (c) Z is C₂₋₈alkenylene-R¹, −Alk-O—R¹, −Alk-S—R¹ or −Alk-NR⁴—R¹ if X is Z;
      and their salts, preferably their pharmaceutically acceptable salts.

C₁₋₈Alkyl and alkoxy radicals may be linear or branched. Examples of C₁₋₈alkyl and alkoxy radicals are methyl, ethyl, n-propyl, isopropyl, n-butyl, isobutyl, sec-butyl, tert-butyl, pentyl, hexyl, and methoxy, ethoxy, propoxy, isopropoxy, butoxy, isobutoxy, sec-butoxy and tert-butoxy. C₁₋₈Alkylenedioxy radicals are preferably methylenedioxy, ethylenedioxy and propylenedioxy. Examples of C₁₋₈alkanoyl radicals are acetyl, propionyl and butyryl. Cycloalkyl is a saturated, cyclic hydrocarbon radical having 3 to 12 carbon atoms, for example cyclopropyl, cyclobutyl, cyclopentyl, cyclohexyl, cycloheptyl, bicyclo[2.2.1]heptyl, cyclooctyl, bicyclo[2.2.2]octyl and adamantyl. Cycloalkyl may be unsubstituted or substituted one or more times, e.g. substituted once or twice by C₁₋₈alkanoyl, C₂₋₈alkenyl, C₂₋₈alkynyl, C₁₋₈alkoxy, C₁₋₈alkoxy-C₁₋₈alkoxy, C₁₋₈alkoxy-C₁₋₈alkyl, C₁₋₈alkoxycarbonylamino, C₁₋₈alkyl, C₀₋₈alkylcarbonylamino, C₁₋₈alkylcarbonyloxy, C₁₋₈alkylenedioxy, optionally N-mono- or N,N-di-C₁₋₈alkylated amino, aryl, optionally N-mono- or N,N-di-C₁₋₈alkylated carbamoyl, optionally esterified carboxy, cyano, C₃₋₈cycloalkoxy, halogen, heteroaryl, heterocyclyl, hydroxy, oxo, polyhalo-C₁₋₈alkoxy or polyhalo-C₁₋₈alkyl. C₁₋₈Alkylene radicals may be linear or branched and are, for example, methylene, ethylene, propylene, 2-methylpropylene, 2-methylbutylene, 2-methylpropyl-2-ene, butyl-2-ene, butyl-3-ene, propyl-2-ene, tetra-, penta- and hexa-methylene; C₂₋₈alkenylene radicals are, for example, vinylene and propenylene; C₂₋₈alkynylene radicals is, for example, ethynylene; acyl radicals are alkanoyl radicals, preferably C₁₋₈alkanoyl radicals, or aroyl radicals such as benzoyl. Aryl refers to mono- or polynuclear aromatic radicals which may be substituted one or more times, such as, for example, phenyl, substituted phenyl, naphthyl, substituted naphthyl, tetrahydronaphthyl or substituted tetrahydronaphthyl. Examples of substituents on such aryl radicals are C₁₋₈alkyl, polyhalo-C₁₋₈alkoxy, polyhalo-C₁₋₈alkyl, nitro, amino, C₁₋₈alkenyl, C₁₋₈alkoxy, C₁₋₈alkyl-carbonyloxy, hydroxy, halogen, cyano, carbamoyl, carboxy and C₁₋₈alkylenedioxy, and optionally halogen-, C₁₋₈alkyl-, C₁₋₈alkoxy- or dihydroxy-C₁₋₈alkylaminocarbonyl-substituted phenyl, phenoxy, phenylthio, phenyl-C₁₋₈alkyl or phenyl-C₁₋₈alkoxy. Further examples of substituents on aryl or heterocyclyl radicals are C₁₋₈alkoxycarbonylphenyl, hydroxy-C₁₋₈alkyl-phenyl, benzyloxy, pyridylcarbonylamino-C₁₋₈alkyl, C₂₋₈alkenyloxy, C₁₋₈alkoxy-C₁₋₈alkoxy, C₁₋₈alkoxy-C₁₋₈alkoxy-C₁₋₈alkyl, methoxybenzyloxy, hydroxybenzyloxy, phenethyloxy, methylenedioxybenzyloxy, dioxolanyl-C₁₋₈alkoxy, cyclopropyl-C₁₋₈alkyl, cyclopropyl-C₁₋₈alkoxy, hydroxy-C₁₋₈alkoxy, carbamoyloxy-C₁₋₈alkoxy, pyridylcarbamoyloxy-C₁₋₈alkoxy, benzoyloxy-C₁₋₈alkoxy, C₁₋₈alkoxycarbonyl, C₀₋₈alkylcarbonylamino, C₀₋₈alkylcarbonylamino-C₁₋₈alkyl, C₀₋₈alkylcarbonylamino-C₁₋₈alkoxy, (N—C₁₋₈alkyl)-C₀₋₈alkylcarbonylamino-C₁₋₈alkyl, (N—C₁₋₈alkyl)-C₀₋₈alkylcarbonylamino-C₁₋₈alkoxy, C₃₋₈-cycloalkylcarbonylamino-C₁₋₈alkyl, C₃₋₈cycloalkylcarbonylamino-C₁₋₈alkoxy, C₁₋₈alkoxy-C₁₋₈alkyl, hydroxy-C₁₋₈alkyl, hydroxy-C₁₋₈alkoxy-C₁₋₈alkyl, hydroxy-C₁₋₈alkoxy-C₁₋₈alkoxy, C₁₋₈alkoxycarbonylamino-C₁₋₈alkyl, C₁₋₈alkoxycarbonylamino-C₁₋₈alkoxy, C₁₋₈alkylaminocarbonylamino-C₁₋₈alkyl, C₁₋₈alkyl-aminocarbonylamino-C₁₋₈alkoxy, C₁₋₈alkylaminocarbonyl-C₁₋₈alkyl, C₁₋₈alkylaminocarbonyl-C₁₋₈alkoxy, C₁₋₈alkylaminocarbonyl-C₁₋₈alkoxy-C₁₋₈alkyl, di-C₁₋₈alkylaminocarbonyl-C₁₋₈alkyl, di-C₁₋₈alkylaminocarbonyl-C₁₋₈alkoxy, C₁₋₈alkylcarbonyloxy-C₁₋₈alkyl, C₁₋₈alkylcarbonyloxy-C₁₋₈alkoxy, cyano-C₁₋₈alkyl, cyano-C₁₋₈alkoxy, 2-oxooxazolidinyl-C₁₋₈alkyl, 2-oxooxazolidinyl-C₁₋₈alkoxy, C₁₋₈alkoxycarbonyl-C₁₋₈alkyl, C₁₋₈alkoxycarbonyl-C₁₋₈alkoxy, C₁₋₈alkylsulfonyl-amino-C₁₋₈alkyl, C₁₋₈alkylsulfonylamino-C₁₋₈alkoxy, (N—C₁₋₈alkyl)-C₁₋₈alkylsulfonylamino-C₁₋₈alkyl, (N—C₁₋₈alkyl)-C₁₋₈alkylsulfonylamino-C₁₋₈alkoxy, C₁₋₈alkylamino-C₁₋₈alkyl, C₁₋₈alkyl-amino-C₂₋₈alkoxy, di-C₁₋₈alkylamino-C₁₋₈alkyl, di-C₁₋₈alkylamino-C₂₋₈alkoxy, C₁₋₈alkylsulfonyl-C₁₋₈alkyl, C₁₋₈alkylsulfonyl-C₁₋₈alkoxy, carboxy-C₁₋₈alkyl, carboxy-C₁₋₈alkoxy, carboxy-C₁₋₈alkoxy-C₁₋₈alkyl, C₁₋₈alkoxy-C₁₋₈alkylcarbonyl, acyl-C₁₋₈alkoxy-C₁₋₈alkyl, (N—C₁₋₈alkyl)-C₁₋₈alkoxycarbonylamino, (N-hydroxy)-C₁₋₈alkylaminocarbonyl-C₁₋₈alkyl, (N-hydroxy)-C₁₋₈alkylaminocarbonyl-C₁₋₈alkoxy, (N-hydroxy)aminocarbonyl-C₁₋₈alkyl, (N-hydroxy)amino-carbonyl-C₁₋₈alkoxy, C₁₋₈alkoxyaminocarbonyl-C₁₋₈alkyl, 6-alkoxyaminocarbonyl-C₁₋₈alkoxy, (N—C₁₋₈alkoxy)-C₁₋₈alkylaminocarbonyl-C₁₋₈alkyl, (N—C₁₋₈alkoxy)-C₁₋₈alkylaminocarbonyl-C₁₋₈alkoxy, (N-acyl)-C₁₋₈alkoxy-C₁₋₈alkylamino, C₁₋₈alkoxy-C₁₋₈alkylcarbamoyl, (N—C₁₋₈alkyl)-C₁₋₈alkoxy-C₁₋₈alkylcarbamoyl, C₁₋₈alkoxy-C₁₋₈alkylcarbonyl, C₁₋₈alkoxy-C₁₋₈alkylcarbonyl-amino, (N—C₁₋₈alkyl)-C₁₋₈alkoxy-C₁₋₈alkylcarbonylamino, 1-C₁₋₈alkoxy-C₁₋₈alkylimidazol-2-yl, 1-C₁₋₈alkoxy-C₁₋₈alkyltetrazol-5-yl, 5-C₁₋₈alkoxy-C₁₋₈alkyltetrazol-1-yl, 2-C₁₋₈alkoxy-C₁₋₈alkyl-4-oxoimidazol-1-yl, carbamoyl-C₁₋₈alkyl, carbamoyl-C₁₋₈alkoxy, C₁₋₈alkylcarbamoyl, di-C₁₋₈alkylcarbamoyl, C₁₋₈alkylsulfonyl, C₁₋₈alkylamidinyl, acetamidinyl-C₁₋₈alkyl, O-methyl-oximyl-C₁₋₈alkyl, O,N-dimethylhydroxylamino-C₁₋₈alkyl, C₃₋₈cycloalkyl-C₁₋₈alkanoyl, aryl-C₁₋₈alkanoyl, heterocyclyl-C₁₋₈alkanoyl; and optionally halogen-, C₁₋₈alkyl-, C₁₋₈alkoxy- or dihydroxy-C₁₋₈alkylaminocarbonyl-substituted pyridyl, pyridyloxy, pyridylthio, pyridylamino, pyridyl-C₁₋₈alkyl, pyridyl-C₁₋₈alkoxy, pyrimidinyl, pyrimidinyloxy, pyrimidinylthio, pyrimidinyl-amino, pyrimidinyl-C₁₋₈alkyl, pyrimidinyl-C₁₋₈alkoxy, thienyl, thienyl-C₁₋₈alkyl, thienyl-C₁₋₈alkoxy, furyl, furyl-C₁₋₈alkyl, furyl-C₁₋₈alkoxy.

The term heterocyclyl refers to mono-, bi- or polycyclic, saturated and unsaturated hetero-cyclic radicals having 1 to 4 nitrogen and/or 1 or 2 sulfur or oxygen atoms, which may be substituted one or more times, in particular once, twice or three times. The term heterocyclyl further encompasses the above oxo-substituted radicals. Heterocyclyl radicals which comprise a nitrogen atom may be linked either via the N atom or via a C atom to the remainder of the molecule.

Examples of unsaturated heterocyclyl radicals are benzo[1,3]dioxolyl, benzofuranyl, benzoimidazolyl, benzooxazolyl, benzothiazolyl, benzo[b]thienyl, quinazolinyl, quinolyl, quinoxalinyl, chromenyl, dihydrobenzofuranyl, 1,3-dihydrobenzoimidazolyl, 3,4-dihydro-2H-benzo[1,4]oxazinyl, dihydro-3H-benzo[1,4]oxazinyl, 1,4-dihydrobenzo[d][1,3]oxazinyl, dihydro-2H-benzo[1,4]thiazinyl, 3,4-dihydro-1H-quinazolinyl, 3,4-dihydro-1H-quinolinyl, 2,3-dihydroindolyl, dihydro-1H-pyrido[2,3-b][1,4]oxazinyl, 1,1-dioxodihydro-2H-benzo[1,4]thiazinyl, furyl, imidazolyl, imidazo[1,5-a]pyridinyl, imidazo[1,2-a]pyrimidinyl, indazolyl, indolyl, isobenzofuranyl, isoquinolyl, [1,5]naphthyridyl, oxazolyl, 1-oxidopyridyl, 2-oxobenzoimidazolyl, 3-oxo-4H-benzo[1,4]oxazinyl, 2-oxobenzooxazolyl, 3-oxo-4H-benzo[1,4]thiazinyl, 2-oxo-1H-quinolinyl, 2-oxo-chromenyl, 2-oxodihydrobenzo[e][1,4]-diazepinyl, 2-oxo-1,3-dihydrobenzoimidazole, 2-oxodihydrobenzo[d][1,3]oxazinyl, 2-oxo-3,4-dihydro-1H-quinazolinyl, 2-oxo-3,4-dihydro-1H-quinolinyl, 4-oxo-dihydroimidazolyl, 2-oxo-1,3-dihydroindolyl, 1-oxo-3H-isobenzofuranyl, 2-oxo-1H-pyrido[2,3-b][1,4]oxazinyl, 2-oxo-1,3,4,5-tetrahydrobenzo[b]azepinyl, 2-oxotetrahydrobenzo[e][1,4]diazepinyl, 4-oxo-3H-thieno[2,3-d]pyrimidinyl, 5-oxo-4H-[1,2,4]triazinyl, C₁₋₈alkylenedioxy-substituted phenyl, phthalazinyl, pyranyl, pyrazinyl, pyrazolyl, pyridyl, pyrimidinyl, 1H-pyrrolizinyl, pyrrolo[3,2-c]pyridinyl, pyrrolo[2,3-c]pyridinyl, pyrrolo[3,2-b]pyridinyl, 1H-pyrrolo[2,3-b]pyridyl, pyrrolyl, 1,3,4,5-tetrahydrobenzo[b]azepinyl, tetrahydroquinolinyl, tetrahydroquinoxalinyl, tetrahydroisoquinolinyl, thiazolyl, thienyl, triazinyl, triazolyl, 1,1,3-trioxodihydro-2H-1/A6-benzo[1,4]thiazinyl, [1,2,3]triazolo[1,5-a]pyridinyl or [1,2,4]triazolo[4,3-a]pyridinyl.

The term saturated heterocyclyl refers to 3-16-membered, mono-, bi- or polycyclic saturated heterocyclic radicals having 1 to 4 nitrogen and/or 1 or 2 sulfur or oxygen atoms. Preference is given to 3-8-membered, particularly preferably 5- or 6-membered, monocyclic radicals which optionally have a 3-8-membered fused-on ring which may be carbocyclic or hetero-cyclic. A further preferred group of heterocyclic radicals are bi- or polycyclic heterocycles which optionally have a spirocyclic or bridged ring. Preferred heterocyclic radicals have in each ring 1 nitrogen, oxygen or sulfur atom, 1-2 nitrogen atoms and 1-2 oxygen atoms or 1-2 nitrogen atoms and 1-2 sulfur atoms, with at least one, preferably 1-7, carbon atoms being present in each ring.

Examples of saturated heterocyclyl radicals are azepanyl, azetidinyl, aziridinyl, 3,4-dihydroxypyrrolidinyl, 2,6-dimethylmorpholinyl, 3,5-dimethylmorpholinyl, dioxanyl, [1,4]dioxepanyl, dioxolanyl, 4,4-di-oxothiomorpholinyl, dithianyl, dithiolanyl, 2-hydroxymethylpyrrolidinyl, 4-hydroxypiperidinyl, 3-hydroxypyrrolidinyl, 4-methylpiperazinyl, 1-methylpiperidinyl, 1-methylpyrrolidinyl, morpholinyl, oxathianyl, oxepanyl, 2-oxoazepanyl, 2-oxoimidazolidinyl, 2-oxooxazolidinyl, 2-oxopiperidinyl, 4-oxopiperidinyl, 2-oxopyrrolidinyl, 2-oxotetrahydropyrimidinyl, 4-oxothiomorpholinyl, piperazinyl, piperidinyl, pyrrolidinyl, tetrahydrofuranyl, tetrahydropyranyl, tetrahydrothiophenyl, tetrahydrothiopyranyl, thiepanyl or thiomorpholinyl.

Examples of bi- or polycyclic heterocyclyl radicals are 2,5-dioxabicyclo[4.1.0]heptanyl, 2-oxa-bicyclo[2.2.1]heptanyl, 2-oxabicyclo[4.1.0]heptanyl, 3-oxabicyclo[4.1.0]heptanyl, 7-oxa-bicyclo[2.2.1]heptanyl, 2-oxabicyclo[3.1.0]hexanyl, 3-oxabicyclo[3.1.0]hexanyl, 1-oxa-spiro[2.5]octanyl, 6-oxaspiro[2.5]octanyl, 3-oxabicyclo[3.3.1]nonanyl, 2-oxo-1a,7b-dihydro-1H-cyclopropa[c]chromenyl or 1,1a,2,7b-tetrahydrocyclopropa[c]chromenyl.

Heterocyclyl may be unsubstituted or substituted one or more times, e.g. once or twice, by C₁₋₈alkanoyl, C₂₋₈alkenyl, C₂₋₈alkynyl, C₁₋₈alkoxy, C₁₋₈alkoxy-C₁₋₈alkoxy, C₁₋₈alkoxy-C₁₋₈alkyl, C₁₋₈alkoxycarbonylamino, C₁₋₈alkyl, C₀₋₈alkylcarbonylamino, C₁₋₈alkylcarbonyloxy, C₁₋₈alkylenedioxy, optionally N-mono- or N,N-di-C₁₋₈alkylated amino, aryl, optionally N-mono- or N,N-di-C₁₋₈alkylated carbamoyl, optionally esterified carboxy, cyano, C₃₋₈cycloalkoxy, halogen, heteroaryl, heterocyclyl, hydroxy, nitro, oxide, oxo, polyhalo-C₁₋₈alkoxy or polyhalo-C₁₋₈alkyl.

The aryl and heterocyclyl radicals in the case of R¹ may additionally be substituted also by heterocyclylalkyl, heterocyclylalkoxy, heterocyclylalkoxyalkyl or heterocyclyl such as, for example, piperidinoalkyl, piperidinoalkoxy, piperidinoalkoxyalkyl, morpholinoalkyl, morpholinoalkoxy, morpholinoalkoxyalkyl, piperazinoalkyl, piperazinoalkoxy, piperazino-alkoxyalkyl, [1,2,4]-triazol-1-ylalkyl, [1,2,4]-triazol-1-ylalkoxy, [1,2,4]-triazol-4-yl-alkyl, [1,2,4]-triazol-4-ylalkoxy, [1,2,4]-oxadiazol-5-ylalkyl, [1,2,4]-oxadiazol-5-ylalkoxy, 3-methyl-[1,2,4]-oxadiazol-5-ylalkyl, 3-methyl-[1,2,4]-oxadiazol-5-ylalkoxy, 5-methyl-[1,2,4]-oxadiazol-3-ylalkyl, 5-methyl-[1,2,4]-oxadiazol-3-ylalkoxy, tetrazol-1-ylalkyl, tetrazol-1-ylalkoxy, tetrazol-2-ylalkyl, tetrazol-2-ylalkoxy, tetrazol-5-ylalkyl, tetrazol-5-ylalkoxy, 5-methyl-tetrazol-1-ylalkyl, 5-methyltetrazol-1-ylalkoxy, thiazol-4-ylalkyl, thiazol-4-ylalkoxy, oxazol-4-ylalkyl, oxazol-4-ylalkoxy, 2-oxopyrrolidinylalkyl, 2-oxopyrrolidinylalkoxy, imidazolylalkyl, imidazolylalkoxy, 2-methylimidazolylalkyl, 2-methylimidazolylalkoxy N-methylpiperazinoalkyl, N-methyl-piperazinoalkoxy, N-methylpiperazinoalkoxyalkyl, and also alkylaminoalkyl, alkylaminoalkoxy, alkylaminoalkoxyalkyl, mono- and polyhydroxyalkyl, mono- and polyhydroxyalkoxy, mono- and polyhydroxyalkoxyalkyl, mono- and polyhydroxyalkoxyalkoxy, carbamoylalkyloxy, C₁₋₈alkoxy, amino-C₁₋₈alkoxy, hydroxy-C₁₋₈alkoxy, dioxolanyl, dioxanyl, dithiolanyl, dithianyl, pyrrolidinyl, piperidinyl, piperazinyl, pyrrolyl, 4-methylpiperazinyl, morpholinyl, thiomorpholinyl, 2-hydroxymethylpyrrolidinyl, 3-hydroxypyrrolidinyl, 3,4-dihydroxypyrrolidinyl, 3-acetamidomethylpyrrolidinyl, 3-C₁₋₈alkoxy-C₁₋₈alkylpyrrolidinyl, 4-hydroxypiperidinyl, 4-oxopiperidinyl, 3,5-dimethylmorpholinyl, 4,4-dioxothiomorpholinyl, 4-oxothiomorpholinyl, 2,6-dimethylmorpholinyl, 2-oxoimidazolidinyl, 2-oxooxazolidinyl, 2-oxopyrrolidinyl, 2-oxo[1,3]oxazinyl, 2-oxo-tetrahydropyrimidinyl and the like or by the radical —O—CH₂CH(OH)CH₂NRₓ, where NRₓ is a mono- or di-C₁₋₈alkylamino, piperidino, morpholino, piperazino or N-methylpiperazino radical.

Halogen- and/or hydroxy-substituted C₁₋₈alkoxy may be for example hydroxy-C₁₋₈alkoxy or else polyhydroxy-C₁₋₈alkoxy.

The term polyhydroxyalkyl refers to C₁₋₈alkyl radicals which may be substituted by 2-8 hydroxy groups, such as, for example, glyceryl, arabityl, sorbityl etc. An analogous statement applies to radicals derived therefrom, such as polyhydroxy-C₁₋₈alkoxy.

The compounds of the formula (I) have at least three asymmetric carbon atoms and may therefore exist in the form of optically pure diastereomers, mixtures of diastereomers, diastereomeric racemates, mixtures of diastereomeric racemates or as meso compounds. The invention encompasses all these forms. Mixtures of diastereomers, diastereomeric racemates or mixtures of diastereomeric racemates can be fractionated by conventional methods, e.g. by column chromatography, thin-layer chromatography, HPLC and the like.

Salts of compounds with salt-forming groups are in particular acid addition salts, salts with bases or, if a plurality of salt-forming groups is present, optionally also mixed salts or inner salts.

Salts are primarily the pharmaceutically acceptable or non-toxic salts of compounds of the formula (I).

Such salts are formed for example by compounds of the formula (I) having an acidic group, e.g. a carboxy or sulfo group, and are for example their salts with suitable bases, such as non-toxic metal salts derived from metals of group Ia, Ib, IIa and IIb of the Periodic Table of the Elements, e.g. alkali metal, in particular lithium, sodium or potassium salts, alkaline earth metal salts, for example magnesium or calcium salts, furthermore zinc salts or ammonium salts, also salts formed with organic amines such as optionally hydroxy-substituted mono-, di- or trialkylamines, especially mono-, di- or tri-lower-alkylamines, or with quaternary ammonium bases, e.g. methyl-, ethyl-, diethyl- or triethylamine, mono-, bis- or tris(2-hydroxy-lower-alkyl)amines such as ethanol-, diethanol- or triethanolamine, tris(hydroxy-methyl)methylamine or 2-hydroxy-tertiary-butylamine, N,N-di-lower-alkyl-N-(hydroxy-lower-alkyl)amine, such as N,N-dimethyl-N-(2-hydroxyethyl)amine, or N-methyl-D-glucamine, or quaternary ammonium hydroxides such as tetrabutylammonium hydroxide. The compounds of the formula I having a basic group, e.g. an amino group, can form acid addition salts, e.g. with suitable inorganic acids, e.g. hydrohalic acid such as hydrochloric acid, hydrobromic acid, sulfuric acid with replacement of one or both protons, phosphoric acid with replacement of one or more protons, e.g. orthophosphoric acid or metaphosphoric acid, or pyrophosphoric acid with replacement of one or more protons, or with organic carboxylic, sulfonic or phosphonic acids or N-substituted sulfamic acids, e.g. acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid, isonicotinic acid, furthermore amino acids such as, for example, the α-amino acids mentioned hereinabove, and methanesulfonic acid, ethanesulfonic acid, 2-hydroxyethanesulfonic acid, ethane-1,2-disulfonic acid, benzene-sulfonic acid, 4-toluenesulfonic acid, naphthalene-2-sulfonic acid, 2- or 3-phosphoglycerate, glucose 6-phosphate, N-cyclohexylsulfamic acid (to form cyclamates) or with other acidic organic compounds such as ascorbic acid. Compounds of the formula (I) having acidic and basic groups may also form inner salts.

Pharmaceutically unsuitable salts may also be used for isolation and purification.

Preferred compounds according to the invention are those of the general formula (IA) and the salts thereof, preferably the pharmaceutically acceptable salts thereof

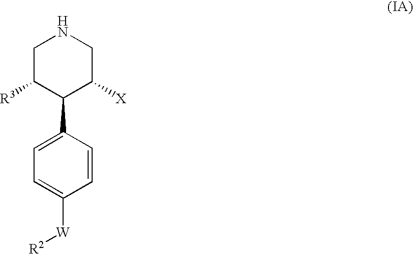

in which R², R³, W and X have the meaning indicated above for the compounds of the formula (I).

A further preferred group of compounds of the formula (I), and particularly preferably of the formula (IA), and the salts thereof, preferably the pharmaceutically acceptable salts thereof, are compounds in which

R¹ is heterocyclyl substituted by oxo or oxide or as indicated under (B) or (C), where heterocyclyl is particularly preferably selected from azepanyl, benzo[1,3]dioxolyl, benzofuranyl, benzoimidazolyl, 4H-benzo[1,4]oxazinyl, benzooxazolyl, 4H-benzo[1,4]thiazinyl, quinolinyl, chromenyl, dihydrobenzo[e][1,4]diazepinyl, dihydrobenzofuranyl, 3,4-dihydro-2H-benzo[1,4]oxazinyl, dihydro-3H-benzo[1,4]oxazinyl, dihydrobenzo[d][1,3]oxazinyl, 3,4-dihydro-2H-benzo[1,4]thiazinyl, dihydro-2H-1λ6-benzo[1,4]thiazinyl, dihydro-1H-quinazolinyl, 1a,7b-dihydro-1H-cyclopropa[c]chromenyl, dihydroimidazolyl, 1,3-dihydroindolyl, 2,3-dihydroindolyl, dihydro-1H-pyrido[2,3-b][1,4]oxazinyl, indazolyl, indolyl, 3H-isobenzofuranyl, [1,5]naphthyridyl, oxazolyl, phthalazinyl, piperidinyl, pyrazolyl, 1H-pyrido[2,3-b][1,4]oxazinyl, pyridyl, 1H-pyrrolizinyl, 1H-pyrrolo[2,3-b]pyridyl, pyrrolyl, tetrahydrobenzo[e][1,4]diazepinyl, 3H-thieno[2,3-d]pyrimidinyl, tetrahydroquinoxalinyl, 1,1a,2,7b-tetrahydro-cyclopropa[c]chromenyl, tetrahydropyranyl or triazinyl.

Particularly preferred radicals R¹ are azepanyl, benzo[1,3]dioxolyl, benzofuranyl, benzo-imidazolyl, 4H-benzo[1,4]oxazinyl, benzooxazolyl, 4H-benzo[1,4]thiazinyl, quinolinyl, chromenyl, dihydrobenzo[e][1,4]diazepinyl, dihydrobenzofuranyl, 3,4-dihydro-2H-benzo[1,4]oxazinyl, dihydro-3H-benzo[1,4]oxazinyl, dihydrobenzo-[d][1,3]oxazinyl, 3,4-dihydro-2H-benzo[1,4]thiazinyl, dihydro-2H-1λ6-benzo[1,4]thiazinyl, dihydro-1H-quinazolinyl, 1a,7b-dihydro-1H-cyclopropa[c]chromenyl, dihydroimidazolyl, 1,3-dihydroindolyl, 2,3-dihydro-indolyl, dihydro-1H-pyrido[2,3-b][1,4]oxazinyl, indazolyl, indolyl, 3H-isobenzofuranyl, [1,5]naphthyridyl, oxazolyl, phthalazinyl, piperidinyl, pyrazolyl, 1H-pyrido[2,3-b][1,4]oxazinyl, pyridyl, 1H-pyrrolizinyl, 1H-pyrrolo[2,3-b]pyridyl, pyrrolyl, tetrahydrobenzo[e][1,4]diazepinyl, 3H-thieno[2,3-d]pyrimidinyl, tetrahydroquinoxalinyl, 1,1a,2,7b-tetrahydro-cyclopropa[c]chromenyl, tetrahydropyranyl or triazinyl substituted by 1-3 C₁₋₈alkanoyl, C₁₋₈alkoxy, C₁₋₈alkoxy-C₁₋₈alkoxy, C₁₋₈alkoxy-C₁₋₈alkoxy-C₁₋₈alkyl, C₁₋₈alkoxy-C₁₋₈alkyl, (N—C₁₋₈alkoxy)-C₁₋₈alkylaminocarbonyl-C₁₋₈alkoxy, (N—C₁₋₈alkoxy)-C₁₋₈alkylaminocarbonyl-C₁₋₈alkyl, C₁₋₈alkoxy-C₁₋₈alkylcarbonyl, C₁₋₈alkoxycarbonylamino-C₁₋₈alkoxy, C₁₋₈alkoxy-carbonylamino-C₁₋₈alkyl, C₁₋₈alkyl, (N—C₁₋₈alkyl)-C₀₋₈alkylcarbonylamino-C₁₋₈alkoxy, (N—C₁₋₈alkyl)-C₀₋₈alkylcarbonylamino-C₁₋₈alkyl, C₀₋₈alkylcarbonylamino-C₁₋₈alkoxy, C₀₋₈alkyl-carbonylamino-C₁₋₈alkyl, halogen, oxide, oxo, polyhalo-C₁₋₈alkoxy, polyhalo-C₁₋₈alkyl, [1,2,4]-triazol-1-ylalkyl, [1,2,4]-triazol-4-ylalkyl, tetrazol-1-ylalkyl, tetrazol-2-ylalkyl, tetrazol-5-ylalkyl or thiazol-4-ylalkyl.

R¹ is very particularly preferably chromenyl, 3,4-dihydro-2H-benzo[1,4]oxazinyl, 3,4-dihydro-2H-benzo[1,4]thiazinyl or 1,3-dihydroindolyl substituted by 1-3 C₁₋₈alkoxy, C₁₋₈alkoxy-C₁₋₈alkoxy, C₁₋₈alkoxy-C₁₋₈alkoxy-C₁₋₈alkyl, C₁₋₈alkoxy-C₁₋₈alkyl, C₁₋₈alkoxy-C₁₋₈alkylcarbonyl, C₁₋₈alkoxycarbonylamino-C₁₋₈alkoxy, C₁₋₈alkoxycarbonylamino-C₁₋₈alkyl, C₁₋₈alkyl, (N—C₁₋₈alkyl)-C₀₋₈alkylcarbonylamino-C₁₋₈alkoxy, (N—C₁₋₈alkyl)-C₀₋₈alkylcarbonylamino-C₁₋₈alkyl, C₀₋₈alkylcarbonylamino-C₁₋₈alkoxy, C₀₋₈alkylcarbonylamino-C₁₋₈alkyl, halogen, oxo, polyhalo-C₁₋₈alkoxy or polyhalo-C₁₋₈alkyl.

A further preferred group of compounds of the formula (I), or particularly preferably of the formula (IA), and the salts thereof, preferably the pharmaceutically acceptable salts thereof, are compounds in which
R¹ has the meaning as indicated for (A) or (B), particularly preferably as indicated for (A);
R² has the meaning as indicated for (a) or (b);
R³ has the meaning as indicated for (a) or (b);
R⁴ is C₁₋₈alkyl or hydrogen;
R⁵ is C₁₋₈alkyl or hydrogen;
R⁶ is C₁₋₈alkyl or hydrogen;
X is —CHR⁶-Alk-R¹, −Alk-NR⁴—R¹, −Alk-O—R¹, −Alk-S—R¹, C₂₋₈-Alkenylen-R¹, —CH(OR⁴)-Alk-R¹, —CHR⁶-Alk-R¹, —CHR⁶—O—R¹, —CHR⁶—O—-Alk-R¹, —CHR⁶—S—R¹, —CHR⁶—S-Alk-R¹, —CO-Alk-R¹, —C(═NOR⁵)-Alk-R¹, —O-Alk-NR⁴—R¹, —O-Alk-R¹, —O-Alk-O—R¹, —O—CO—R¹, —O—CO-Alk-R¹, —O—CHR⁶—R¹, —O—CHR⁶-Alk-R¹, —O—CHR⁶—CO—NR⁴—R¹ or —O—CHR⁶—CO—NR⁴-Alk-R¹, where Alk is C₁₋₈alkylene; and
W is —O—, —S— or cyano.

A further preferred group of compounds of the formula (I), or particularly preferably of the formula (IA), and the salts thereof, preferably the pharmaceutically acceptable salts thereof, are compounds in which
R¹ has the meaning as indicated for (A) or (B), particularly preferably as indicated for (A);
R² has the meaning as indicated for (a) or (b);
R³ has the meaning as indicated for (a) or (b);
R⁴ is C₁₋₈alkyl or hydrogen;
R⁵ is C₁₋₈alkyl or hydrogen;
R⁶ is C₁₋₈alkyl or hydrogen;
X is —CHR⁶-Alk-R¹, —CH(OR⁴)-Alk-R¹, —O-Alk-R¹, —O—CHR⁶—R¹ or —O—CHR⁶—CO—NR⁴—R¹, where Alk is C₁₋₈alkylene; and
W is —O—, —S— or cyano.

Preference is furthermore given to compounds of the formulae (I) and (IA) and the salts thereof, preferably the pharmaceutically acceptable salts thereof, in which X is preferably —CHR⁶-Alk-R¹, —CH(OR⁴)-Alk-R¹, —O-Alk-R¹, —O—CHR⁶—R¹ or —O—CHR⁶—CO—NR⁴—R¹.

Preference is furthermore given to compounds of the formulae (I) and (IA) and the salts thereof, preferably the pharmaceutically acceptable salts thereof, in which
R³ a) is halogen- and/or hydroxy-substituted C₁₋₈alkoxy, halogen- and/or hydroxy-substituted C₁₋₈alkoxy-C₁₋₈alkoxy, branched C₁₋₈alkoxy-C₁₋₈alkoxy, optionally N-mono- or N,N-di-C₁₋₈alkylated amino-C₁₋₈alkoxy, optionally N-mono- or N,N-di-C₁₋₈alkylated amino-C₀₋₈alkyl-carbonyl-C₁₋₈alkoxy, substituted C₃₋₈cycloalkyl-C₀₋₈alkoxy, optionally C₁₋₈alkoxy or hydroxy-substituted heterocyclyl-C₀₋₈alkoxy, heterocyclylcarbonyl-C₀₋₈alkoxy, heterocyclylcarbonyl-C₀₋₈alkyl, optionally halogen-substituted heterocyclyl-C₀₋₈alkyl-carbonylamino-C₁₋₈alkyl, optionally halogen-substituted C₃₋₈cycloalkyl-C₀₋₈alkylcarbonyl-amino-C₁₋₈alkyl or optionally halogen-substituted C₁₋₈alkylcarbonylamino-C₁₋₈alkyl; or additionally
   b) hydroxy, unsubstituted C₁₋₈alkoxy, unsubstituted, unbranched C₁₋₈alkoxy-C₁₋₈alkoxy or unsubstituted C₃₋₈cycloalkyl-C₀₋₈alkoxy if —W—R² is not C₁₋₈alkoxy.
R³ is very particularly preferably
   a) hydroxy-substituted C₁₋₈alkoxy, optionally hydroxy-substituted C₁₋₈alkoxy-C₁₋₈alkoxy, branched C₁₋₈alkoxy-C₁₋₈alkoxy or C₁₋₈alkylcarbonylamino-C₁₋₈alkyl; or additionally
   b) hydroxy, unsubstituted C₁₋₈alkoxy or unsubstituted, unbranched C₁₋₈alkoxy-C₁₋₈alkoxy if —W—R² is not C₁₋₈alkoxy.

Preference is furthermore given to compounds of the formulae (I) and (IA) and the salts thereof, preferably the pharmaceutically acceptable salts thereof, in which
R² is C₁₋₈alkyl, C₂₋₈alkenyl, C₁₋₈alkoxy-C₁₋₈alkyl, C₁₋₈alkoxy-C₃₋₈cycloalkyl-C₁₋₈alkyl, C₃₋₈-cyclo-alkyl-C₀₋₈alkoxy-C₁₋₈alkyl-, C₁₋₈alkylsulfanyl-C₁₋₈alkyl, C₁₋₈alkylsulfonyl-C₁₋₈alkyl;
R³ is hydroxy-substituted C₁₋₈alkoxy, optionally hydroxy-substituted C₁₋₈alkoxy-C₁₋₈alkoxy, hydroxy or C₁₋₈alkylcarbonylamino-C₁₋₈alkyl; and
W is —S—.

Preference is furthermore given to compounds of the formulae (I) and (IA) and the salts thereof, preferably the pharmaceutically acceptable salts thereof, in which
R² is C₁₋₈alkyl, C₂₋₈alkenyl, C₁₋₈alkoxy-C₁₋₈alkyl, C₁₋₈alkoxy-C₃₋₈cycloalkyl-C₁₋₈alkyl, C₃₋₈cycloalkyl-C₀₋₈alkoxy-C₁₋₈alkyl-, C₁₋₈alkylsulfanyl-C₁₋₈alkyl, C₁₋₈alkylsulfonyl-C₁₋₈alkyl;
R³ a) hydroxy-substituted C₁₋₈alkoxy, hydroxy-substituted C₁₋₈alkoxy-C₁₋₈alkoxy, branched C₁₋₈alkoxy-C₁₋₈alkoxy or C₁₋₈alkylcarbonylamino-C₁₋₈alkyl; or additionally
   b) hydroxy, unsubstituted C₁₋₈alkoxy or unsubstituted, unbranched C₁₋₈alkoxy-C₁₋₈alkoxy if R² is not C₁₋₈alkyl;
      and
W is —O—.

Particularly preferred radicals R² are C₁₋₈alkyl, C₂₋₈alkenyl, C₁₋₈alkoxy-C₁₋₈alkyl, C₃₋₈cycloalkyl-C₀₋₈alkoxy-C₁₋₈alkyl, C₁₋₈alkylsulfanyl-C₁₋₈alkyl or C₁₋₈alkylsulfonyl-C₁₋₈alkyl when W is —O— or —S—;

Very particular preference is given to compounds and the salts thereof, preferably the pharmaceutically acceptable salts thereof, of the formulae (I) and (IA) in which
R¹ is substituted chromenyl or 3,4-dihydro-2H-benzo[1,4]oxazinyl;
R² is C₁₋₈alkyl, C₂₋₈alkenyl, C₁₋₈alkoxy-C₁₋₈alkyl, C₁₋₈alkoxy-C₃₋₈cycloalkyl-C₁₋₈alkyl, C₃₋₈cycloalkyl-C₀₋₈alkoxy-C₁₋₈alkyl, C₁₋₈alkylsulfanyl-C₁₋₈alkyl;
R³ a) hydroxy-substituted C₁₋₈alkoxy, hydroxy-substituted C₁₋₈alkoxy-C₁₋₈alkoxy, branched C₁₋₈alkoxy-C₁₋₈alkoxy or C₁₋₈alkylcarbonylamino-C₁₋₈alkyl; or additionally
   b) hydroxy, unsubstituted C₁₋₈alkoxy or unsubstituted, unbranched C₁₋₈alkoxy-C₁₋₈alkoxy if R² is not C₁₋₈alkyl;
R⁶ is C₁₋₈alkyl or hydrogen;
X is —CHR⁶-Alk-R¹ or —O-Alk-R¹, where Alk is C₁₋₈alkylene; and
W is —O—.

The groups of compounds mentioned above are not to be regarded as closed; on the contrary, it is possible for parts of these groups of compounds to be interchanged or replaced by the definitions or preferences given, or omitted, in a worthwhile manner, e.g. to replace general by more specific definitions. The definitions and preferences mentioned apply within the scope of general chemical principles such as, for example, the usual valencies of atoms.

The compounds of the formula (I) can be prepared in an analogous manner to preparation processes disclosed in the literature. Similar preparation processes are described for example in WO 97/09311. Details of the specific preparation variants can be found in the examples.

The compounds of the formula (I) can also be prepared in optically pure form. Separation into antipodes can take place by methods known per se, either preferably at an early stage in the synthesis by salt formation with an optically active acid such as, for example, (+)- or (−)-mandelic acid and separation of the diastereomeric salts by fractional crystallization or preferably at a rather late stage by derivatizing with a chiral auxiliary component such as, for example, (+)- or (−)-camphanoyl chloride, and separation of the diastereomeric products by chromatography and/or crystallization and subsequent cleavage of the linkage to the chiral auxiliary. The pure diastereomeric salts and derivatives can be analysed to determine the absolute configuration of the contained piperidine by conventional spectroscopic methods, with X-ray spectroscopy on single crystals representing a particularly suitable method.

The compounds of the formula (I) and (IA) also include compounds in which one or more atoms are replaced by their stable, non-radioactive isotopes; for example a hydrogen atom by deuterium.

Prodrug derivatives of the compounds described herein are derivatives thereof which on in vivo use liberate the original compound by a chemical or physiological process. A prodrug may for example be converted into the original compound when a physiological pH is reached or by enzymatic conversion. Possible examples of prodrug derivatives are esters of freely available carboxylic acids, S- and O-acyl derivatives of thiols, alcohols or phenols, the acyl group being defined as above. Preferred derivatives are pharmaceutically acceptable ester derivatives which are converted by solvolysis in physiological medium into the original carboxylic acid, such as, for example, lower alkyl esters, cycloalkyl esters, lower alkenyl esters, benzyl esters, mono- or disubstituted lower alkyl esters such as lower (amino, mono- or dialkylamino, carboxy, lower alkoxycarbonyl)—alkyl esters or such as lower α-(alkanoyloxy, alkoxycarbonyl or dialkylaminocarbonyl)—alkyl esters; conventionally, pivaloyloxymethyl esters and similar esters are used as such.

Because of the close relationship between a free compound, a prodrug derivative and a salt compound, a particular compound in this invention also includes its prodrug derivative and salt form, where this is possible and appropriate. The definitions mentioned apply within the scope of general chemical principles such as, for example, the usual valencies of atoms.

The compounds of the formula (I) or (IA), and their pharmaceutically acceptable salts have an inhibitory effect on the natural enzyme renin. The latter passes from the kidneys into the blood and there brings about the cleavage of angiotensinogen to form the decapeptide angiotensin I which is then cleaved in the lung, the kidneys and other organs to the octa-peptide angiotensin II. Angiotensin II raises the blood pressure both directly by arterial constriction, and indirectly by releasing the hormone aldosterone, which retains sodium ions, from the adrenals, which is associated with an increase in the extracellular fluid volume. This increase is attributable to the effect of angiotensin II itself or of the heptapeptide angiotensin III formed therefrom as cleavage product. Inhibitors of the enzymatic activity of renin bring about a reduction in the formation of angiotensin I and, as a consequence thereof, the formation of a smaller amount of angiotensin II. The reduced concentration of this active peptide hormone is the direct cause of the blood pressure-lowering effect of renin inhibitors.

The effect of renin inhibitors is detected inter alia experimentally by means of in vitro tests where the reduction in the formation of angiotensin I is measured in various systems (human plasma, purified human renin together with synthetic or natural renin substrate). The following in vitro test of Nussberger et al. (1987) J. Cardiovascular Pharmacol., Vol. 9, pp. 39-44, is used inter alia. This test measures the formation of angiotensin I in human plasma. The amount of angiotensin I formed is determined in a subsequent radio-immunoassay. The effect of inhibitors on the formation of angiotensin I is tested in this system by adding various concentrations of these substances. The IC₅₀ is defined as the concentration of the particular inhibitor which reduces the formation of angiotensin I by 50%. The compounds of the present invention show inhibitory effects in the in vitro systems at minimal concentrations of about 10⁻⁶ to about 10⁻¹⁰ mol/l. Illustrative of the invention, the compounds of examples 1, 2, 5, 8, 61, 68, 70, 71, 73, 74, 87 und 91 inhibit the formation of angiotensin I with IC₅₀ values in the range of about 3.8-814·10⁻⁹ mol/l.

The blood pressure-lowering effect of the compounds described herein can be tested in vivo by the following protocol:

The investigations take place in 5- to 6-week old male doubly transgenic rats (dTGR) which express both human angiotensinogen and human renin and consequently develop hyper-tension. This doubly transgenic rat strain was generated by crossing two strains, the first transgenic for human angiotensinogen with the endogenous promoter and the second transgenic for human renin with the endogenous promoter. Neither of the transgenic strains was previously hypertensive. The doubly transgenic rats, whether male or female, develop severe hypertension (average systolic arterial blood pressure approximately 200 mm Hg) and survive for a median of 55 days. The fact that human renin can be investigated in a rat is a unique feature of this model. Age-matched Sprague-Dawley rats serve as non-hypertensive control animals. The animals are divided into treatment groups and receive each day active compound, comparison substance or vehicle (control) for some weeks. The dose used for oral administration may range from 0.5 to 100 mg/kg of body weight. Throughout the study, the animals received standard feed and tap water ad libitum. The systolic and diastolic blood pressure and the heart rate are measured telemetrically by means of implanted transducers in the abdominal aorta, the animals being able to move freely and unrestrictedly. The effect of the compounds described herein on renal damage (proteinuria) can be tested in vivo by the following protocol:

The investigations take place in 4-week old, male doubly transgenic rats (dTGR) as described above. The animals are divided into treatment groups and receive active compound, comparison substance or vehicle (control) for 7 weeks. The dose used for oral administration may range from 0.5 to 100 mg/kg of body weight. Throughout the study, the animals receive standard feed and tap water ad libitum. The animals are placed periodically in metabolism cages in order to determine the 24-hour albumin excretion in the urine, diuresis, natriuresis and urine osmolality. At the end of the study, the animals are sacrificed and the kidneys and hearts can be removed for weight determination and immunohistological investigations (fibrosis, macrophages/T cell infiltration, etc.).

Renin inhibitors bring about a fall in blood pressure in salt-depleted animals. Human renin differs from renin of other species. Inhibitors of human renin are tested using primates (marmosets, Callithrix jacchus) because human renin and primate renin are substantially homologous in the enzymatically active region. The following in vivo test is employed inter alia: the test compounds are tested on normotensive marmosets of both sexes with a body weight of about 350 g, which are conscious, unrestrained and in their normal cages. Blood pressure and heart rate are measured with a catheter in the descending aorta and are recorded radiometrically. Endogenous release of renin is stimulated by combining a low-salt diet for 1 week with a single intramuscular injection of furosemide (5-(aminosulfonyl)-4-chloro-2-[(2-furanylmethyl)amino]benzoic acid) (5 mg/kg). 16 hours after the furosemide injection, the test substances are administered either directly into the femoral artery by means of a hypodermic needle or as suspension or solution by gavage into the stomach, and their effect on blood pressure and heart rate is evaluated. The compounds of the present invention have a blood pressure-lowering effect in the described in vivo test with i.v. doses of about 0.003 to about 0.3 mg/kg and with oral doses of about 0.3 to about 30 mg/kg.

The pharmacokinetic properties of the compounds described herein can be tested in vivo by the following protocol:

The investigations take place in precatheterized (carotid artery) male rats (300 g±20%) which are able to move unrestrictedly throughout the protocol. The compound is administered intravenously and orally (gavage) to separate animals. The dose used for oral administration may range from 0.5 to 50 mg/kg of body weight; the dose for intravenous administration may range from 0.5 to 20 mg/kg of body weight. Blood samples are taken through the catheter before administration of the compound and during the subsequent 24 hours automatically by means of an AccuSampler (DiLab Europe, Lund, Sweden). The plasma levels of the compounds are then measured using a validated LC-MS analytical method. Subsequently, the pharmacokinetic analysis is undertaken on the basis of the plasma concentration-time plots after determination of the average concentrations at the respective time points. The analysis includes the following parameters: maximum concentration (Cₘₐₓ), time to reach the maximum concentration (tₘₐₓ), area under the curve from 0 hours to the time of the last quantifiable concentration (AUC₀₋ₜ), area under the curve from 0 hours to infinity (AUC_{-inf}), elimination constant (K), terminal half-life (t_{1/2}), absolute oral bioavailability (F), clearance (CL) and volume of distribution during the terminal phase (Vd).

The compounds of the formula (I), and preferably of the formula (IA), and their pharmaceutically acceptable salts can be used as medicines, e.g. in the form of pharmaceutical products. The pharmaceutical products can be administered enterally, such as orally, e.g. in the form of tablets, lacquered tablets, sugar-coated tablets, hard and soft gelatine capsules, solutions, emulsions or suspensions, nasally, e.g. in the form of nasal sprays, rectally, e.g. in the form of suppositories, or transdermally, e.g. in the form of ointments or patches. How-ever, administration is also possible parenterally, such as intramuscularly or intravenously, e.g. in the form of solutions for injection.

Tablets, lacquered tablets, sugar-coated tablets and hard gelatine capsules can be produced by processing the compounds of the formula (I), or preferably of the formula (IA), and their pharmaceutically acceptable salts with pharmaceutically inert inorganic or organic excipients. Excipients of these types which can be used for example for tablets, sugar-coated tablets and hard gelatine capsules are lactose, maize starch or derivatives thereof, talc, stearic acid or salts thereof etc.

Excipients suitable for soft gelatine capsules are, for example, vegetable oils, waxes, fats, semisolid and liquid polyols etc.

Excipients suitable for producing solutions and syrups are, for example, water, polyols, sucrose, invert sugar, glucose etc.

Excipients suitable for solutions for injection are, for example, water, alcohols, polyols, glycerol, vegetable oils, bile acids, lecithin etc.

Excipients suitable for suppositories are, for example, natural or hardened oils, waxes, fats, semiliquid or liquid polyols etc.

The pharmaceutical products may in addition comprise preservatives, solubilizers, viscosity-increasing substances, stabilizers, wetting agents, emulsifiers, sweeteners, colorants, aromatizers, salts to alter the osmotic pressure, buffers, coating agents or antioxidants. They may also comprise other substances of therapeutic value.

The present invention further provides the use of the compounds of the formula (I), or preferably of the formula (IA), and their pharmaceutically acceptable salts in the treatment or prevention of high blood pressure, heart failure, glaucoma, myocardial infarction, renal failure, restenoses and stroke.

The compounds of the formula (I), and preferably of the formula (IA), and their pharmaceutically acceptable salts can also be administered in combination with one or more agents having cardiovascular activity, e.g. α- and β-blockers such as phentolamine, phenoxy-benzamine, prazosin, terazosin, tolazine, atenolol, metoprolol, nadolol, propranolol, timolol, carteolol etc.; vasodilators such as hydralazine, minoxidil, diazoxide, nitroprusside, flosequinan etc.; calcium antagonists such as aminone, bencyclan, diltiazem, fendiline, flunarizine, nicardipine, nimodipine, perhexyline, verapamil, gallopamil, nifedipine etc.; ACE inhibitors such as cilazapril, captopril, enalapril, lisinopril etc.; potassium activators such as pinacidil; antiserotoninergics such as ketanserine; thromboxane synthetase inhibitors; neutral endopeptidase inhibitors (NEP inhibitors); angiotensin II antagonists; and diuretics such as hydrochlorothiazide, chlorothiazide, acetazolamide, amiloride, bumetanide, benzthiazide, ethacrynic acid, furosemide, indacrinone, metolazone, spironolactone, triamterene, chlorthalidone etc.; sympatholytics such as methyldopa, clonidine, guanabenz, reserpine; and other agents suitable for the treatment of high blood pressure, heart failure or vascular disorders associated with diabetes or renal disorders such as acute or chronic renal failure in humans and animals. Such combinations can be used separately or in products which comprise a plurality of components.

Further substances which can be used in combination with the compounds of the formulae (I), (IA) or their pharmaceutically acceptable salts are the compounds of classes (i) to (ix) on page 1 of WO 02/40007 (and the preferences and examples detailed further therein) and the substances mentioned on pages 20 and 21 of WO 03/027091.

Thus the present invention further provides pharmaceutical combinations in the form of a product or of a kit composed of individual components consisting a) of a compound of the general formula (I) or of its pharmaceutical acceptable salt, according to any one of claims 1 to 6, and b) at least one pharmaceutical form as active ingredient having a cardiovascular effect.

The dosage may vary within wide limits and must of course be adapted to the individual circumstances in each individual case. In general, a daily dose appropriate for oral administration ought to be from about 3 mg to about 3 g, preferably about 10 mg to about 1 g, e.g. approximately 300 mg per adult person (70 kg), divided into preferably 1-3 single doses, which may be for example of equal size, although the stated upper limit may also be exceeded if this proves to be indicated, and children usually receive a reduced dose appropriate for their age and body weight.

### EXAMPLES

The following examples illustrate the present invention. All temperatures are stated in degrees Celsius and pressures in mbar. Unless mentioned otherwise, the reactions take place at room temperature. The abbreviation “Rf=xx (A)” means for example that the Rf xx was found in solvent system A. The ratio of amounts of solvents to one another is always indicated in proportions by volume. Chemical names for final products and intermediates were generated with the aid of the AutoNom 2000 (Automatic Nomenclature) program.

| | | | R_{f} | Rt |
|---|---|---|---|---|
| No. | Structure | Appearance | (System) | (Method) |
| 1 | 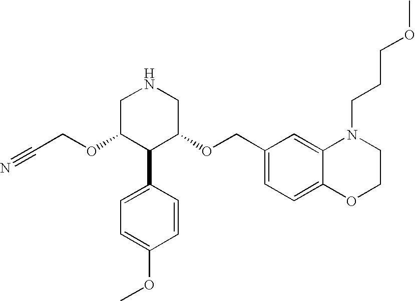 | yellowish resin | 0.31 (C) | 3.62 (I) |
| 2 | 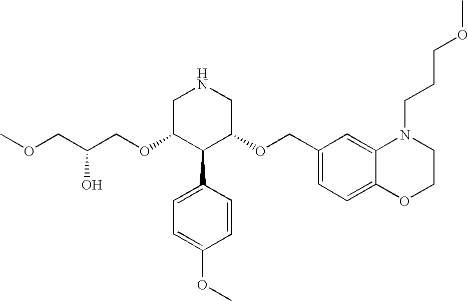 | yellowish oil | 0.17 (A) | 3.33 (I) |
| 3 | 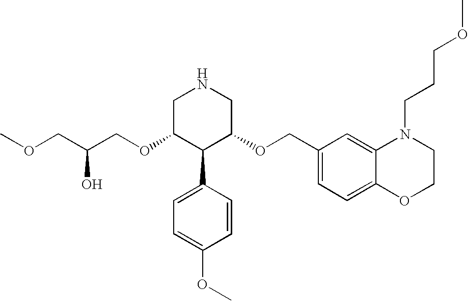 | yellowish oil | 0.17 (A) | 3.30 (I) |
| 5 | 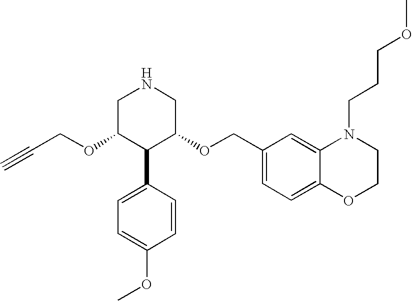 | yellowish resin | 0.21 (C) | 3.72 (I) |
| 6 | 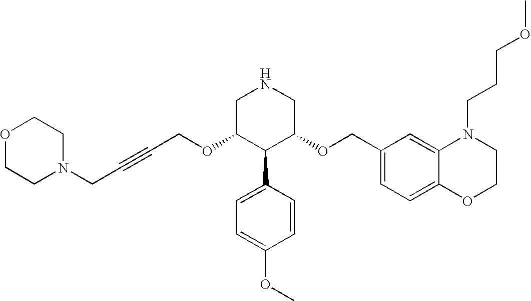 | yellowish resin | 0.28 (C) | 3.15 (I) |
| 7 | 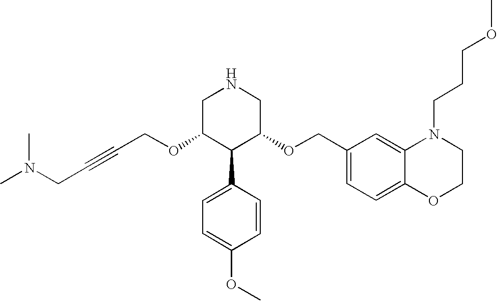 | violet oil | 0.09 (J) | 3.13 (I) |
| 8 | 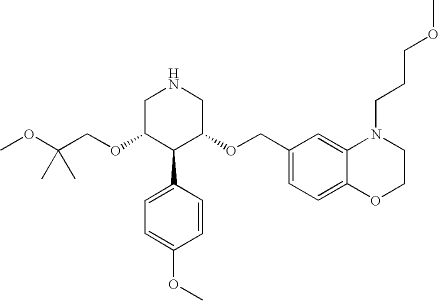 | yellowish oil | 0.1 (C) | 3.79 (I) |
| 9 | 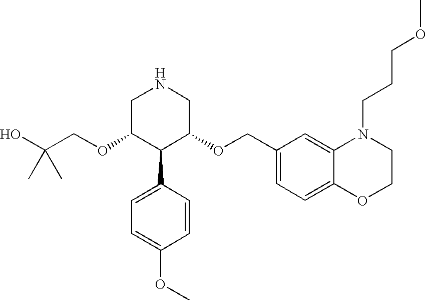 | yellowish oil | 0.22 (C) | 3.51 (I) |
| 10 | 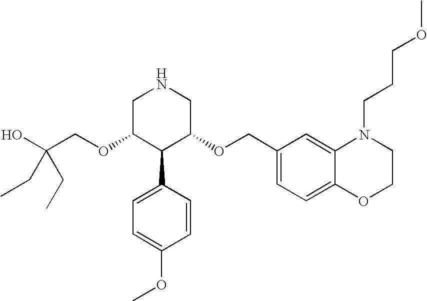 | yellow oil | 0.15 (C) | 3.85 (I) |
| 11 | 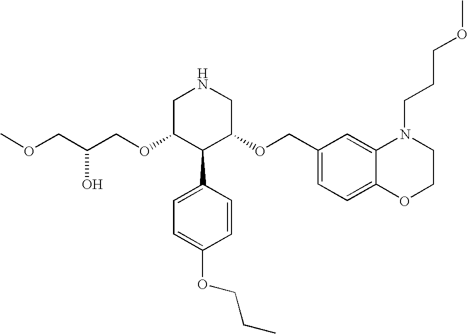 | colourless oil | 0.12 (C) | 3.83 (I) |
| 12 | 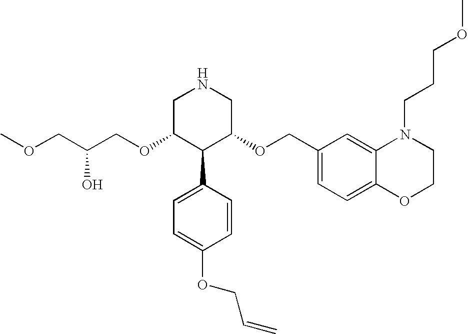 | yellowish oil | 0.31 (A) | 3.66 (I) |
| 13 | 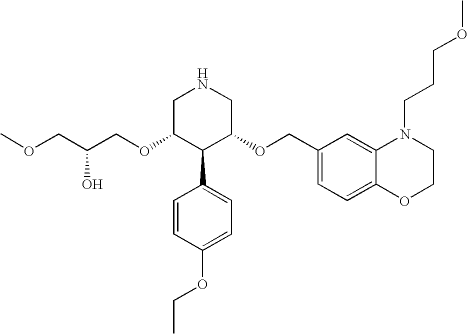 | pale yellow cloudy resin | 0.194 (A) | 3.55 (I) |
| 14 | 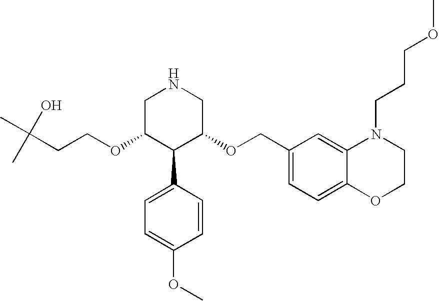 | yellow oil | 0.13 (C) | 3.51 (I) |
| 16 | 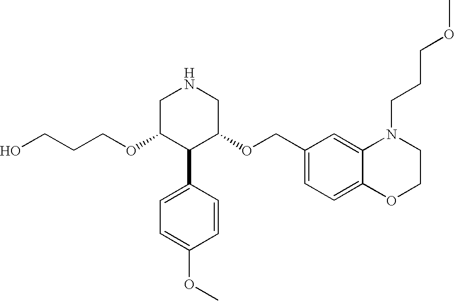 | yellowish oil | 0.08 (C) | 3.29 (I) |
| 17 | 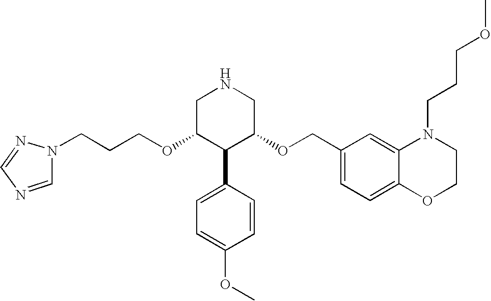 | cloudy oil | 0.08 (J) | 3.25 (I) |
| 18 | 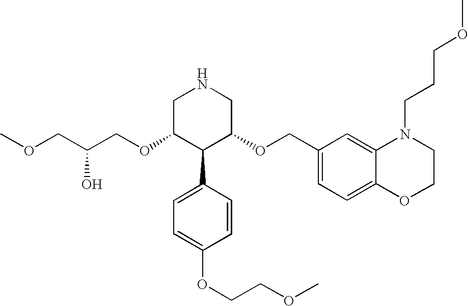 | yellowish oil | 0.33 (A) | 3.31 (I) |
| 19 | 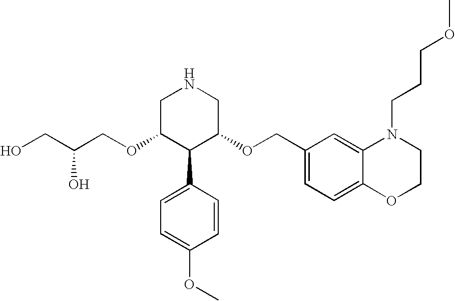 | colourless wax | 0.22 (A) | 3.11 (I) |
| 20 | 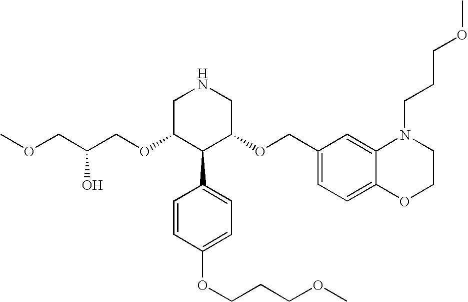 | yellow resin | 0.25 (A) | 3.50 (I) |
| 21 | 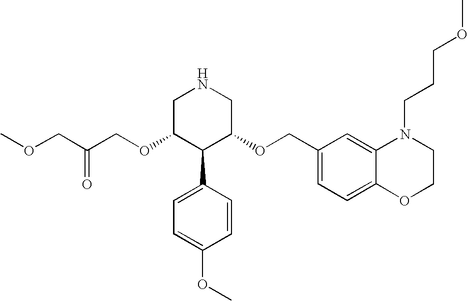 | dark yellow oil | 0.22 (A) | 3.49 (I) |
| 22 | 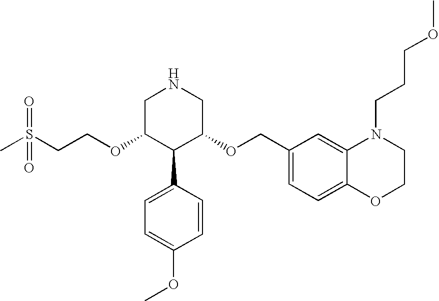 | yellowish resin | 0.08 (C) | 3.40 (I) |
| 23 | 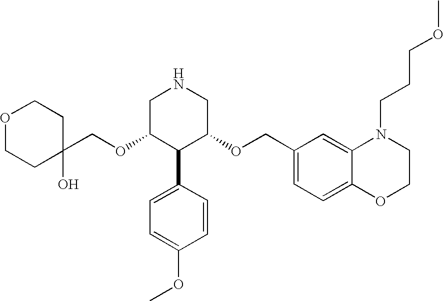 | yellowish resin | 0.19 (A) | 3.32 (I) |
| 24 | 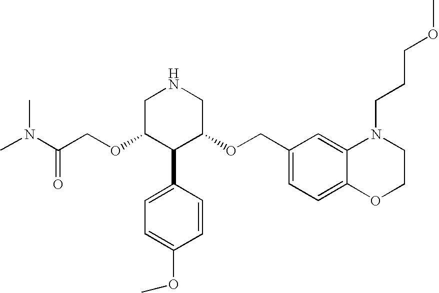 | yellowish, cloudy oil | 0.1 (C) | 3.41 (I) |
| 25 | 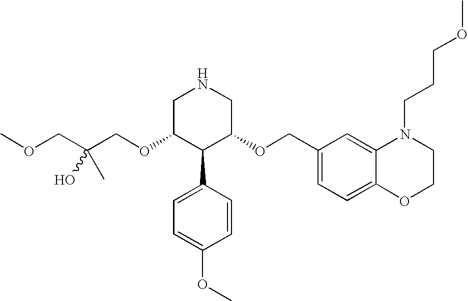 | yellowish oil | 0.27 (A) | 3.52 (I) |
| 26 | 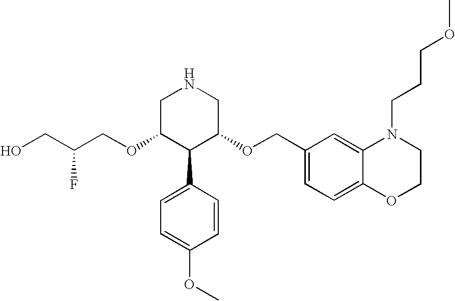 | yellow wax | 0.25 (A) | 3.36 (I) |
| 27 | 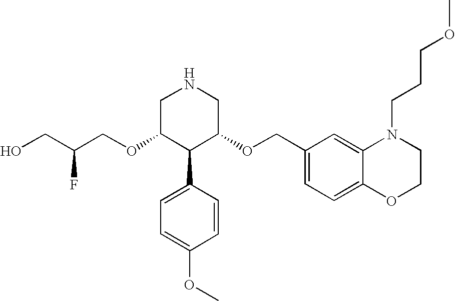 | yellow wax | 0.26 (A) | 3.35 (I) |
| 28 | 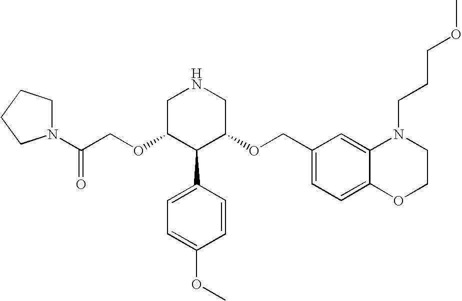 | yellow oil | 0.61 (H) | 3.56 (I) |
| 29 | 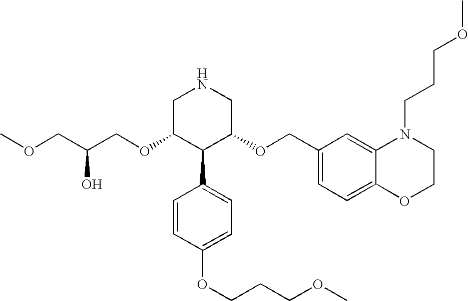 | black oil | 0.25 (A) | 3.47 (I) |
| 30 | 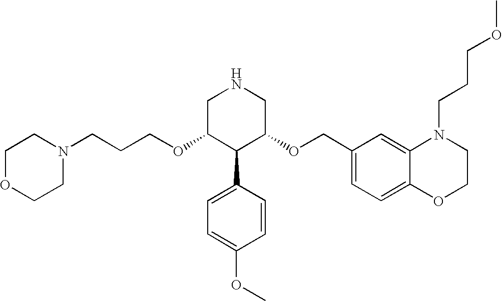 | yellowish resin | 0.37 (A) | 3.05 (I) |
| 31 | 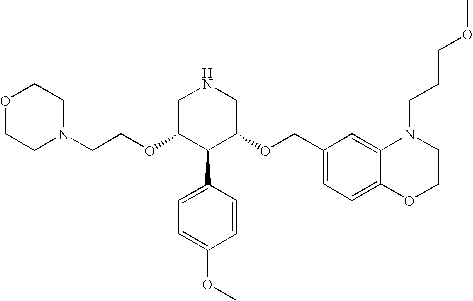 | yellowish resin | 0.37 (A) | 3.04 (I) |
| 32 | 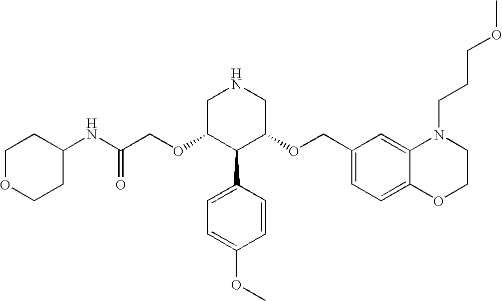 | yellow resin | 0.31 (C) | 3.38 (I) |
| 33 | 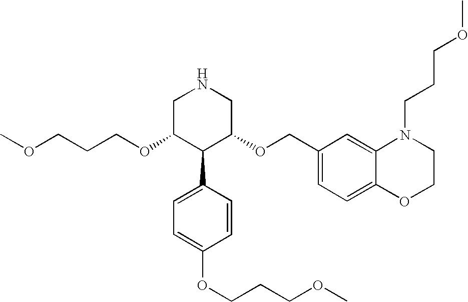 | yellowish liquid | 0.48 (A) | 3.80 (I) |
| 34 | 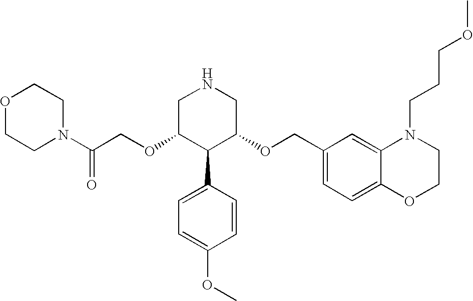 | colourless oil | 0.25 (A) | 3.38 (I) |
| 35 | 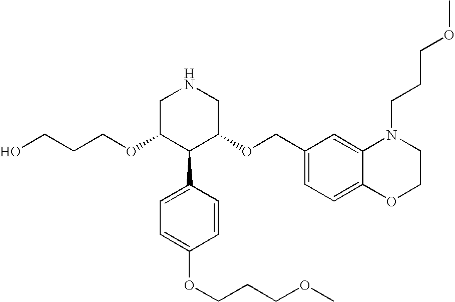 | yellow oil | 0.26 (A) | 3.43 (I) |
| 36 | 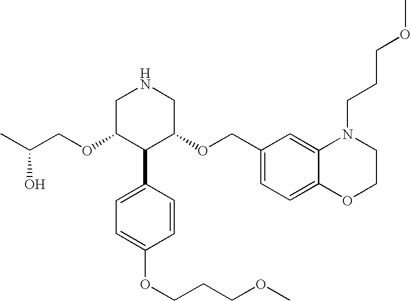 | colourless oil | 0.26 (A) | 3.51 (I) |
| 37 | 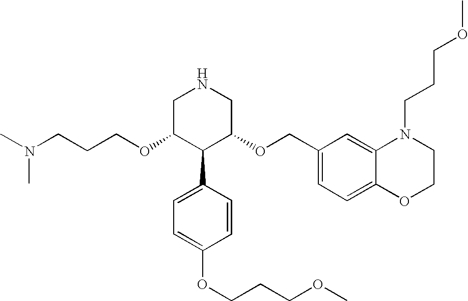 | pale yellowish oil | 0.05 (C) | 3.13 (II) |
| 38 | 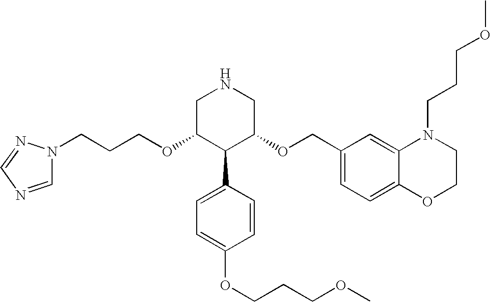 | pale yellowish oil | 0.28 (A) | 3.38 (I) |
| 39 | 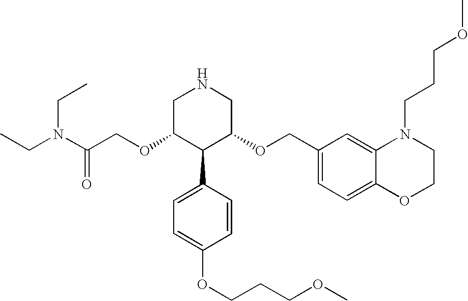 | colourless oil | 0.24 (A) | 3.82 (I) |
| 40 | 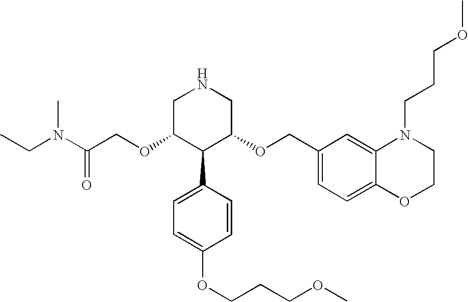 | colourless oil | 0.21 (A) | 3.69 (I) |
| 41 | 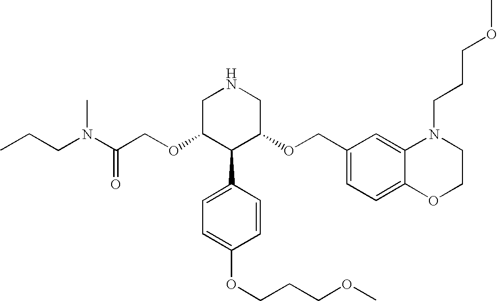 | colourless oil | 0.24 (A) | 3.85 (I) |
| 42 | 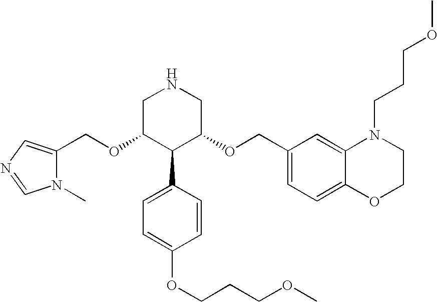 | colourless oil | 0.21 (A) | 3.11 (I) |
| 43 | 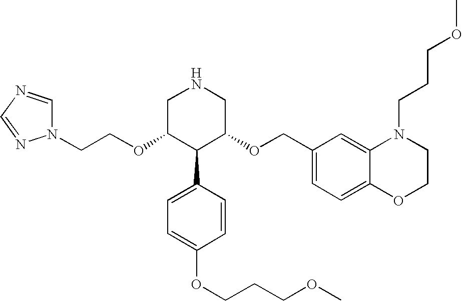 | pale yellow resin | 0.27 (A) | 3.30 (I) |
| 44 | 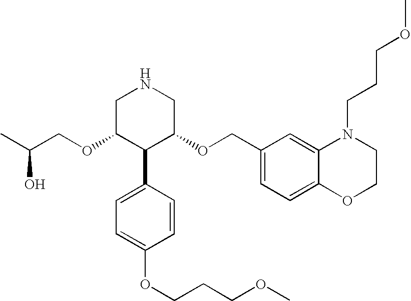 | colourless oil | 0.24 (A) | 3.52 (I) |
| 45 | 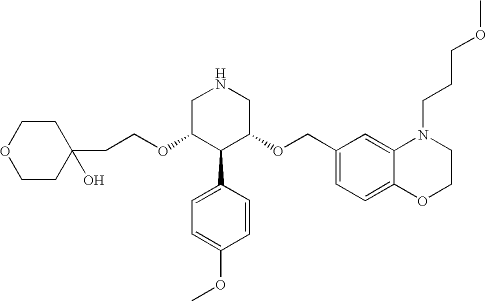 | yellowish oil | 0.25 (A) | 3.32 (I) |
| 46 | 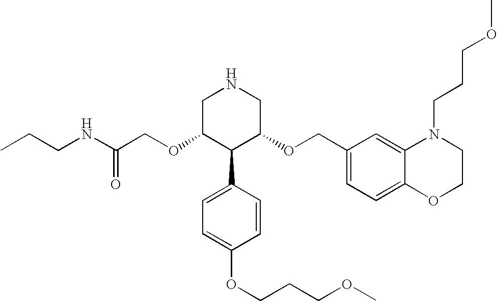 | yellowish resin | 0.44 (G) | 3.79 (II) |
| 47 | 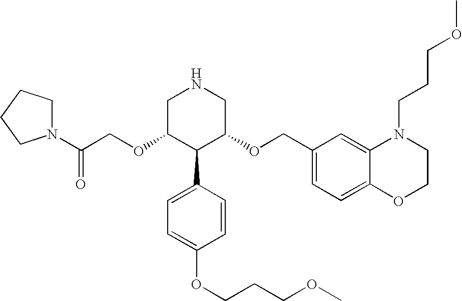 | colourless oil | 0.21 (A) | 3.67 (I) |
| 48 | 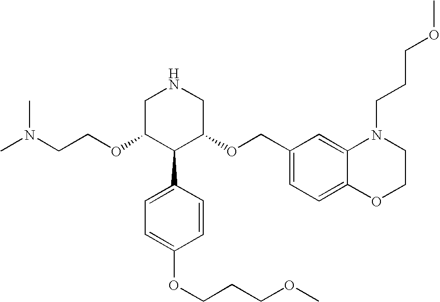 | brown resin | 0.33 (H) | 3.12 (I) |
| 49 | 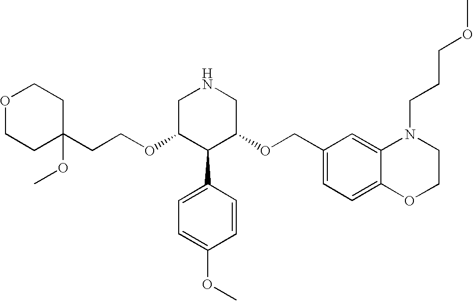 | yellowish oil | 0.1 (C) | 3.57 (I) |
| 50 | 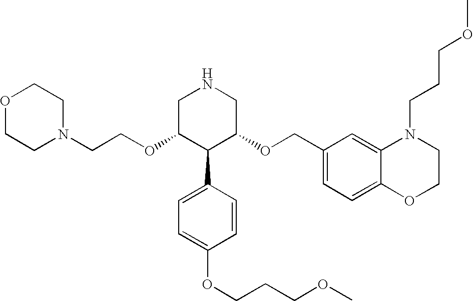 | yellowish oil | 0.29 (I) | 3.19 (I) |
| 51 | 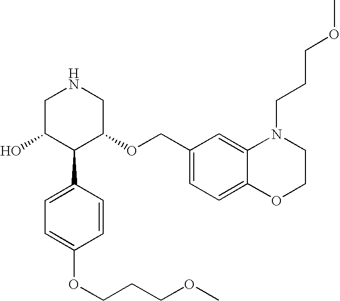 | yellow resin | 0.20 (A) | 3.40 (I) |
| 52 | 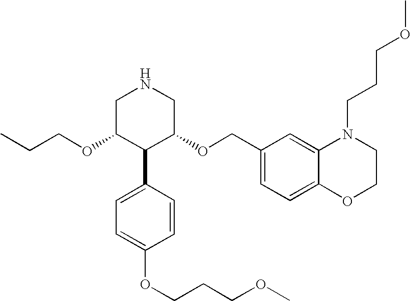 | pale yellow resin | 0.38 (A) | 4.06 (I) |
| 53 | 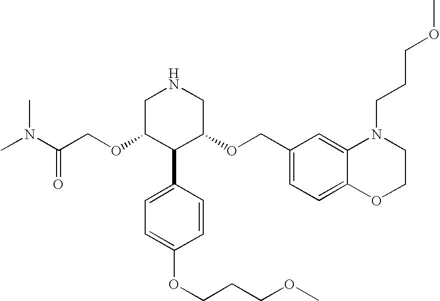 | yellowish oil | 0.31 (I) | 3.57 (I) |
| 54 | 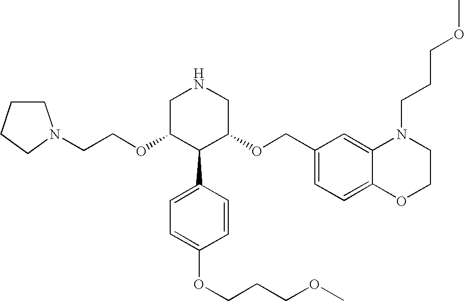 | yellowish oil | 0.12 (I) | 3.24 (I) |
| 55 | 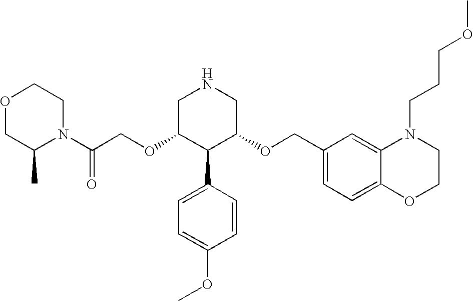 | yellow resin | 0.48 (G) | 3.51 (I) |
| 56 | 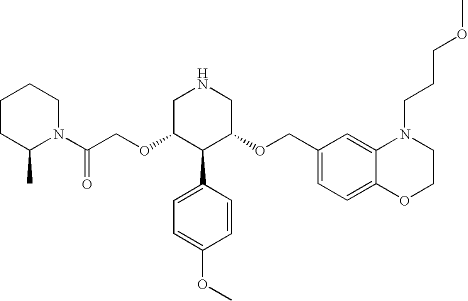 | orange resin | 0.42 (G) | 3.94 (I) |
| 57 | 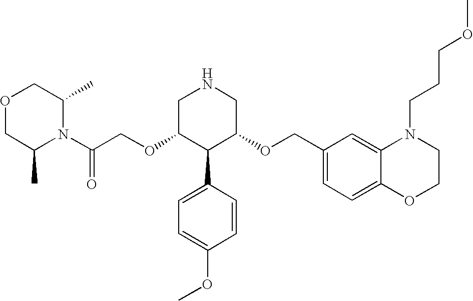 | orange resin | 0.32 (G) | 3.59 (I) |
| 58 | 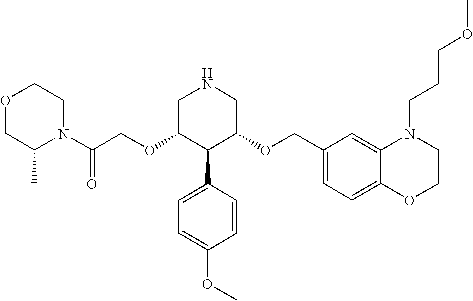 | yellow oil | 0.27 (A) | 3.48 (I) |
| 59 | 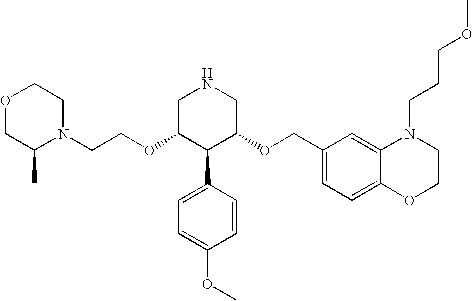 | yellow oil | 0.17 (A) | 3.10 (I) |
| 60 | 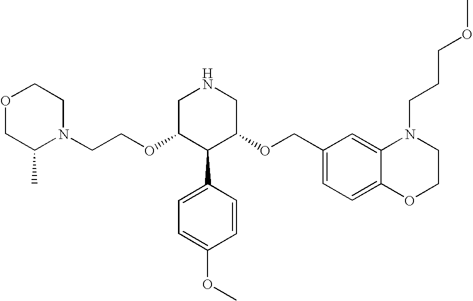 | yellow oil | 0.16 (A) | 3.14 (I) |
| 61 | 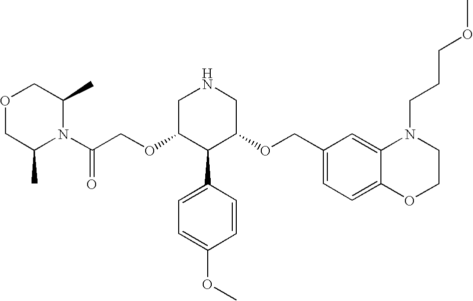 | yellow oil | 0.31 (A) | 3.61 (I) |
| 62 | 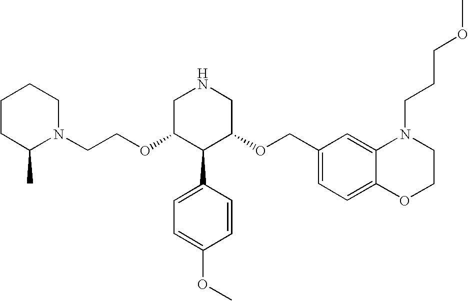 | yellow oil | 0.13 (H) | 3.27 (I) |
| 63 | 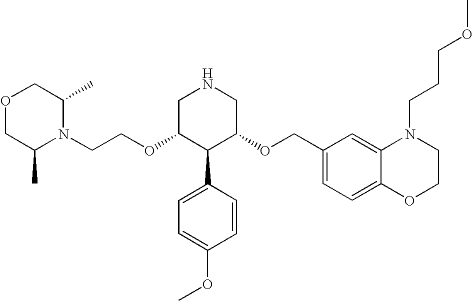 | yellowish resin | 0.25 (D) | 3.17 (I) |
| 64 | 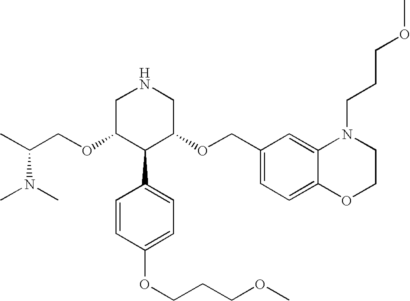 | yellow oil | 0.11 (A) | 3.17 (I) |
| 65 | 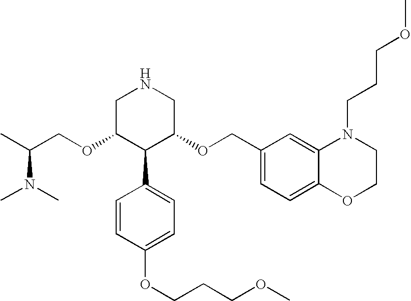 | yellow oil | 0.11 (A) | 3.18 (I) |
| 66 | 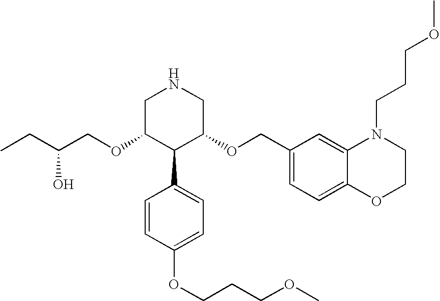 | pale yellow resin | 0.16 (A) | 3.70 (I) |
| 67 | 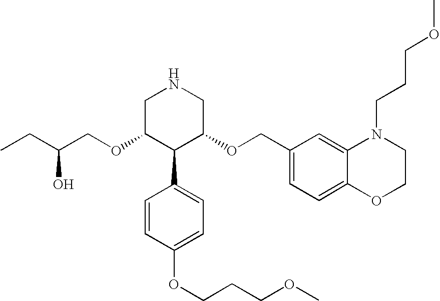 | pale yellow resin | 0.165 (A) | 3.67 (I) |
| 68 | 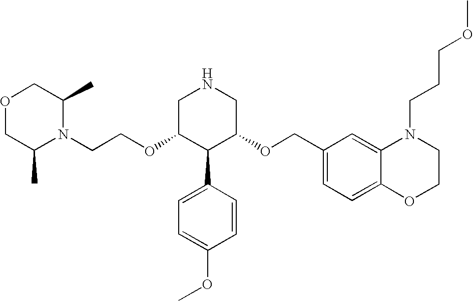 | yellowish resin | 0.17 (G) | 3.18 (I) |
| 69 | 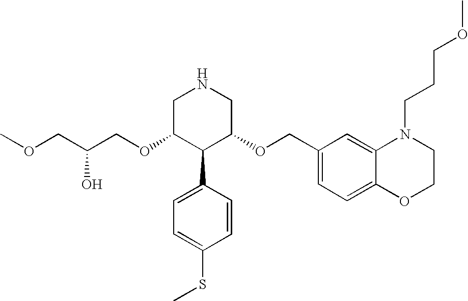 | yellow oil | 0.27 (A) | 3.58 (I) |
| 70 | 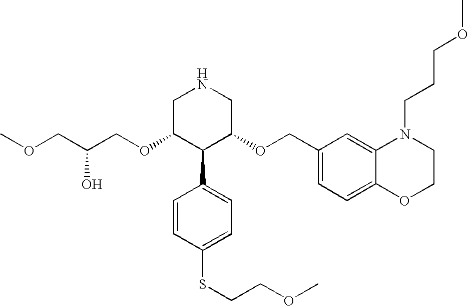 | yellow oil | 0.45 (D) | 3.53 (I) |
| 71 | 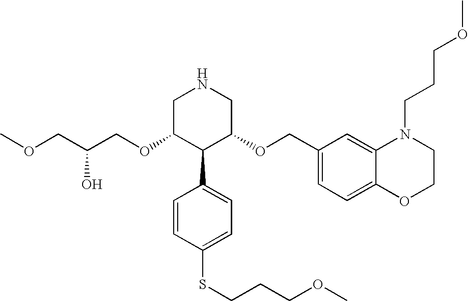 | yellow oil | 0.44 (D) | 3.71 (I) |
| 72 | 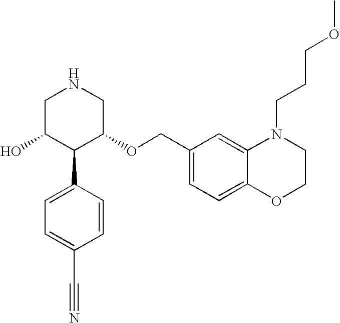 | colourless oil | 0.31 (A) | 3.14 (I) |
| 73 | 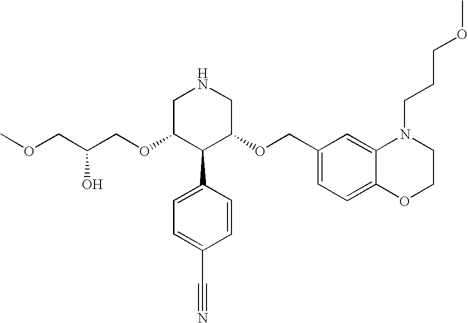 | colourless oil | 0.32 (A) | 3.24 (I) |
| 74 | 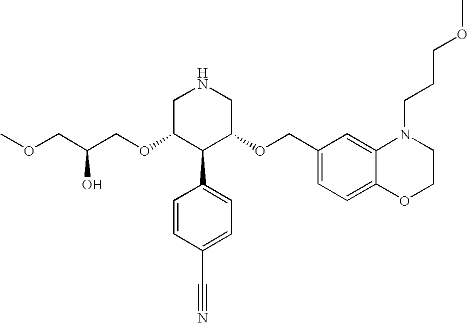 | yellow wax | 0.27 (A) | 3.22 (I) |
| 75 | 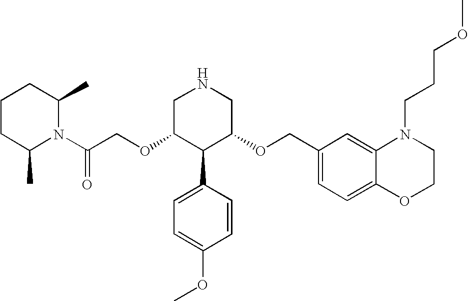 | yellowish resin | 0.38 (D) | 4.09 (I) |
| 76 | 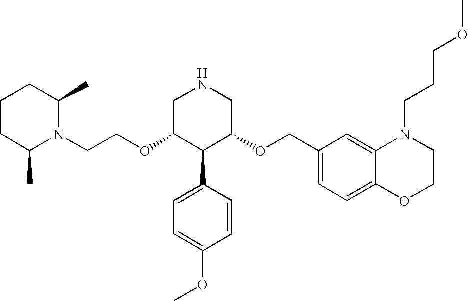 | yellow resin | 0.08 (A) | 3.34 |
| 77 | 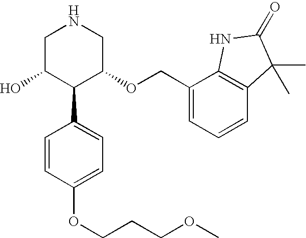 | white foam | 0.21 (A) | 3.17 (I) |
| 78 | 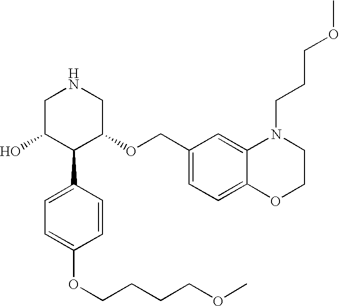 | yellowish oil | 0.08 (C) | 3.56 (I) |
| 79 | 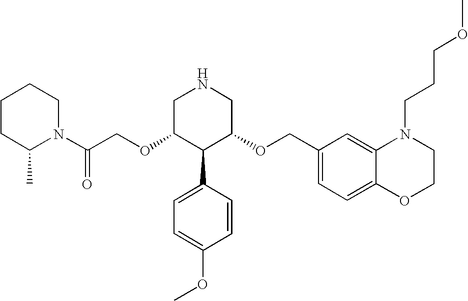 | yellowish resin | 0.35 (G) | 3.93 (I) |
| 80 | 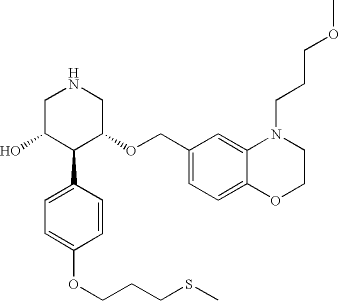 | yellowish resin | 0.18 (G) | 3.79 (I) |
| 81 | 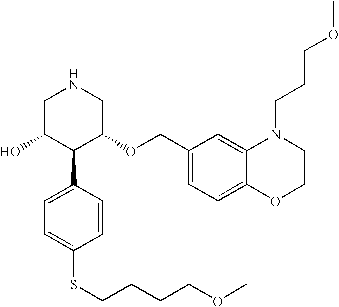 | yellowish oil | 0.22 (A) | 3.76 (I) |
| 82 | 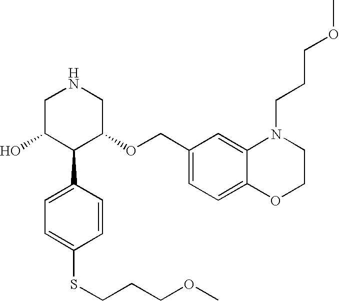 | yellowish resin | 0.26 (A) | 3.63 (I) |
| 83 | 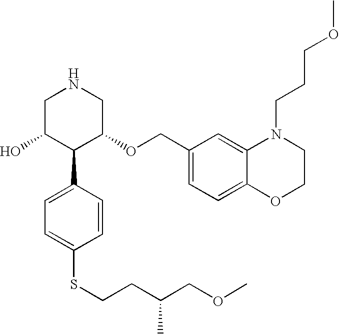 | yellowish oil | 0.22 (A) | 4.01 (I) |
| 87 | 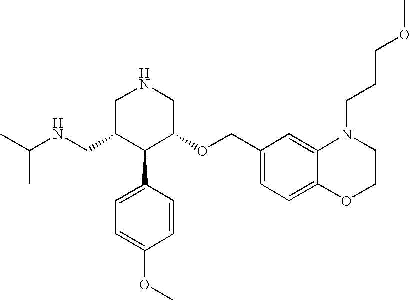 | beige foam | 0.04 (H) | 3.09 (I) |
| 91 | 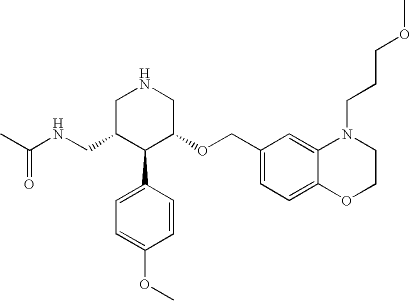 | yellowish foam | 0.71 (K) | 3.30 (I) |
| 92 | 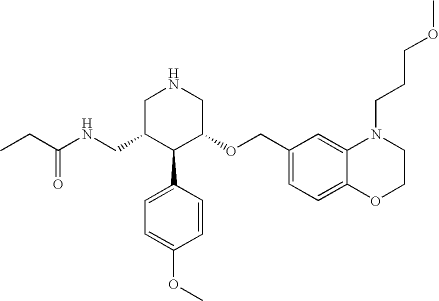 | pale yellow oil | 0.21 (I) | 3.43 (I) |
| 94 | 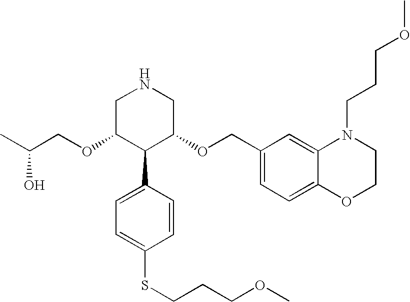 | yellow oil | 0.19 (A) | 3.75 (I) |
| 95 | 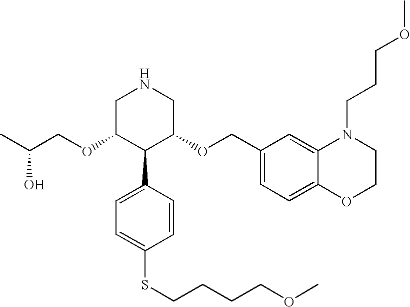 | yellow oil | 0.18 (A) | 3.87 (I) |
| 96 | 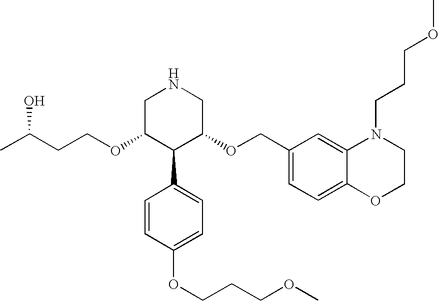 | yellow oil | 0.19 (A) | 3.58 (I) |
| 97 | 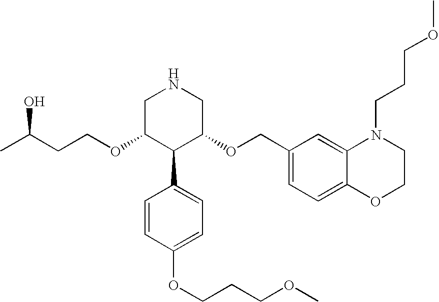 | violet resin | 0.40 (G) | 3.55 (I) |
| 98 | 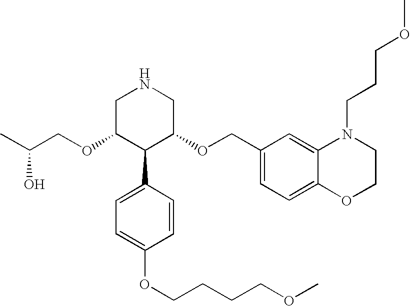 | yellowish oil | 0.27 (A) | 3.65 (I) |
| 99 | 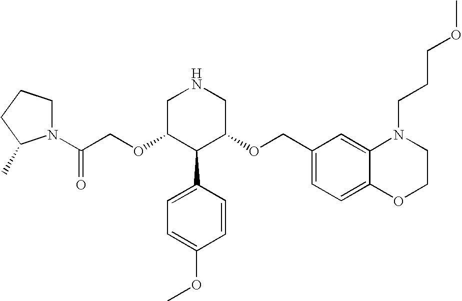 | yellowish resin | 0.39 (G) | 3.74 (I) |
| 100 | 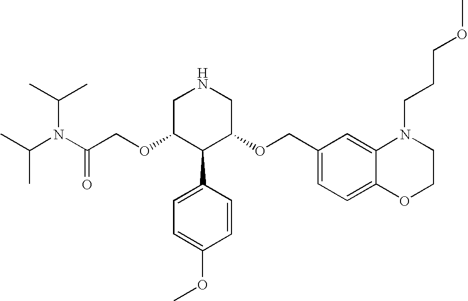 | yellowish resin | 0.44 (G) | 4.12 (I) |
| 101 | 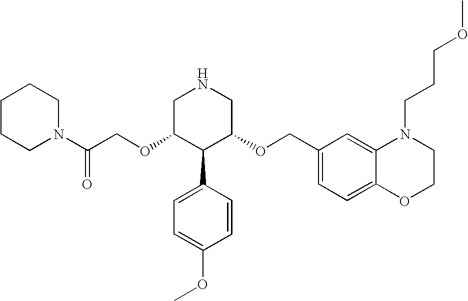 | yellowish oil | 0.40 (G) | 3.80 (I) |
| 102 | 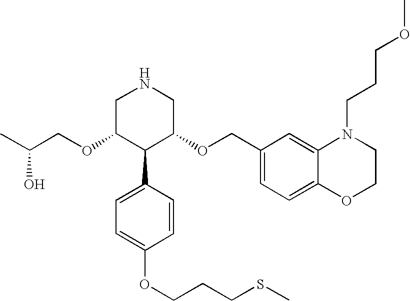 | yellow oil | 0.19 (A) | 3.89 (I) |
| 103 | 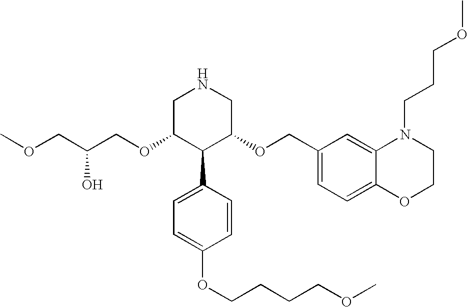 | colourless oil | 0.32 (A) | 3.65 (I) |
| 104 | 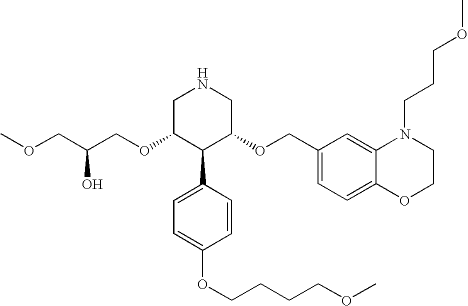 | colourless oil | 0.32 (A) | 3.63 (I) |
| 105 | 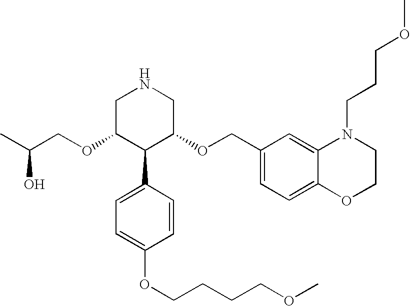 | yellow oil | 0.30 (A) | 3.68 (I) |
| 106 | 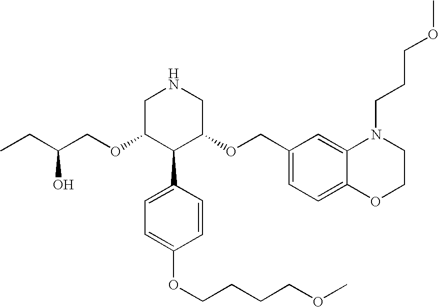 | yellow oil | 0.21 (A) | 3.84 (I) |
| 107 | 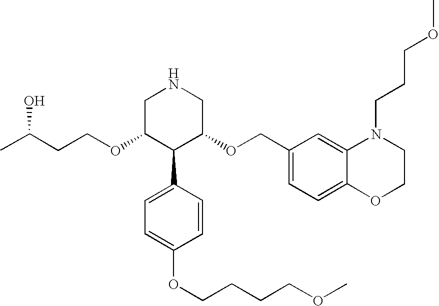 | beige oil | 02.6 (A) | 3.71 (I) |
| 108 | 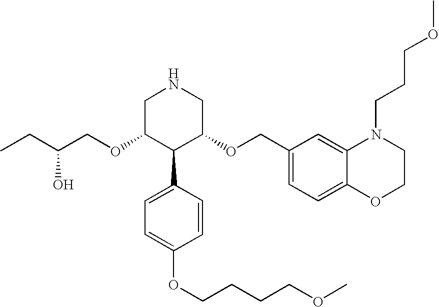 | yellowish oil | 0.25 (A) | 3.86 (I) |
| 109 | 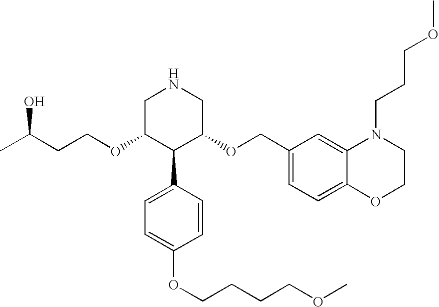 | yellowish oil | 0.45 (A) | 3.70 (I) |
| 110 | 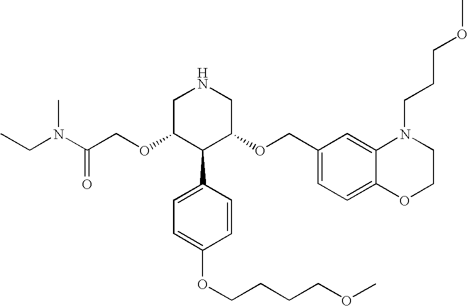 | colourless oil | 0.23 (A) | 3.86 (I) |
| 111 | 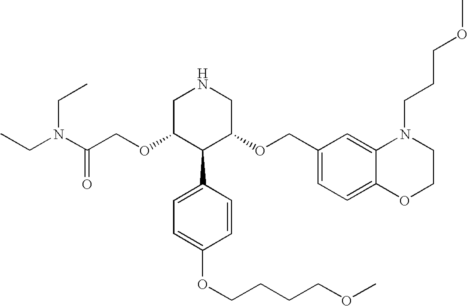 | colourless oil | 0.23 (A) | 3.98 (I) |
| 112 | 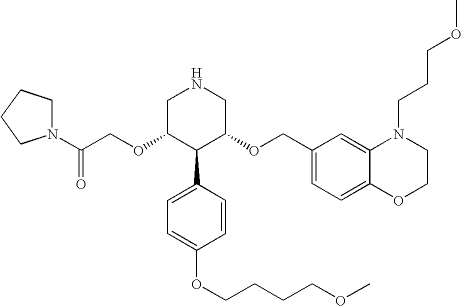 | colourless oil | 0.22 (A) | 3.87 (I) |
| 113 | 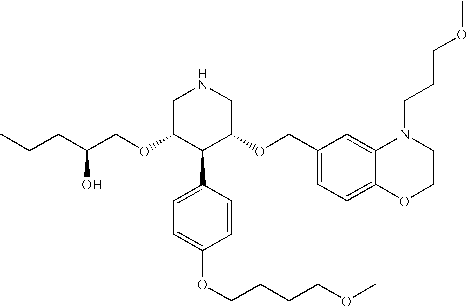 | beige oil | 0.20 (A) | 4.01 (I) |
| 114 | 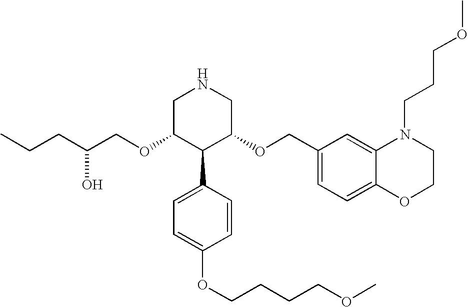 | yellowish oil | 0.41 (A) | 4.04 (I) |
| 116 | 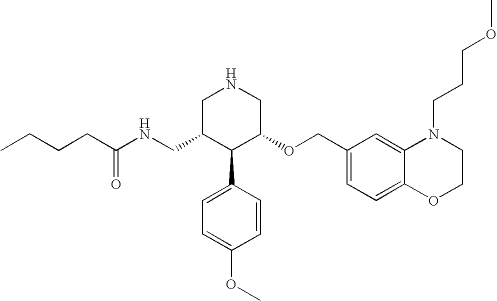 | pale yellow oil | 0.15 (A) | 3.81 (I) |
| 117 | 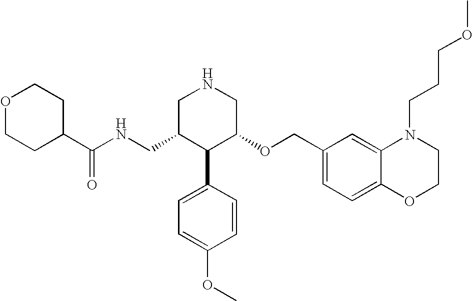 | pale yellow oil | 0.16 (I) | 3.38 (I) |
| 119 | 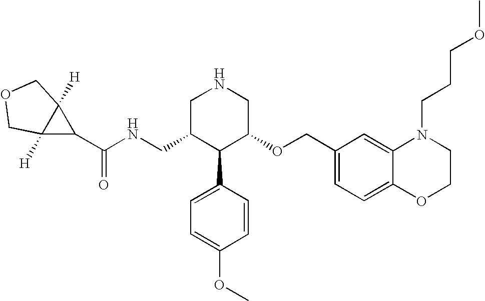 | white foam | 0.15 (I) | 3.42 (I) |
| 122 | 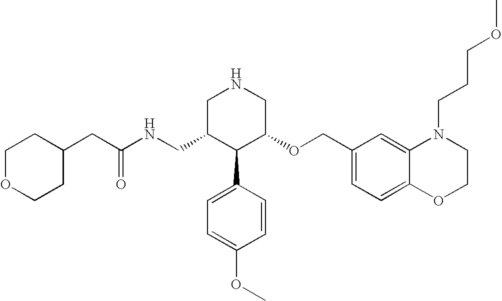 | colourless oil | 0.41 (A) | 3.40 (I) |
| 125 | 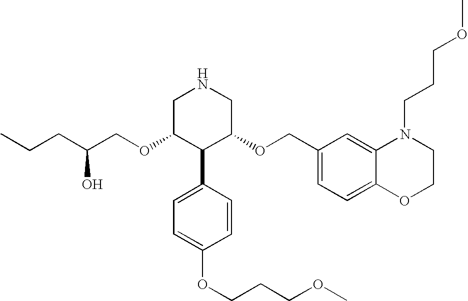 | colorless oil | 0.28 (A) | 3.82 (I) |
| 126 | 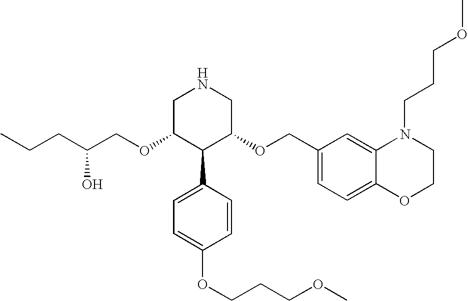 | colorless oil | 0.28 (A) | 3.84 (I) |
| 127 | 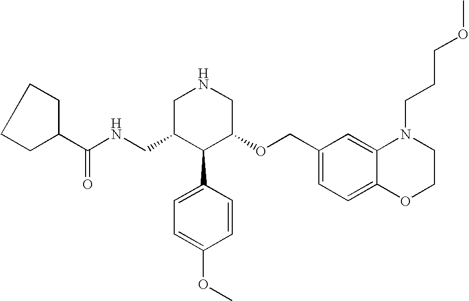 | pale yellow oil | 0.17 (I) | 3.80 (I) |
| 128 | 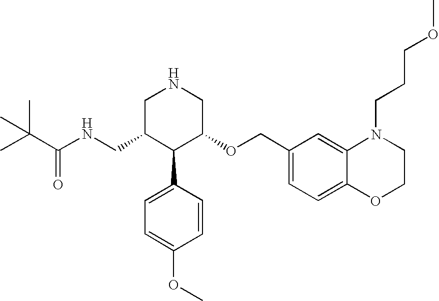 | pale yellow oil | 0.12 (I) | 3.81 (I) |
| 129 | 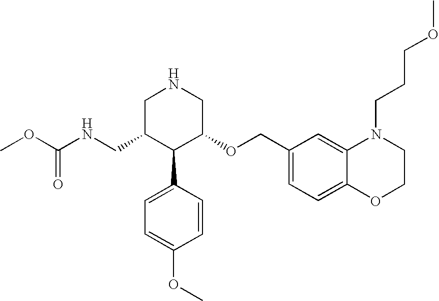 | pale yellow oil | 0.32 (I) | 3.53 (I) |
| 130 | 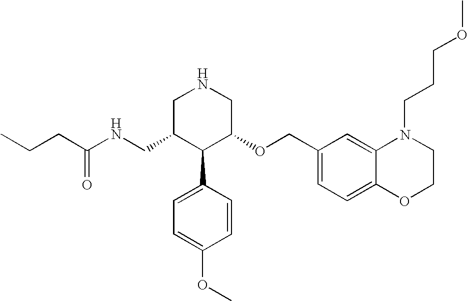 | pale yellow oil | 0.19 (I) | 3.57 (I) |
| 131 | 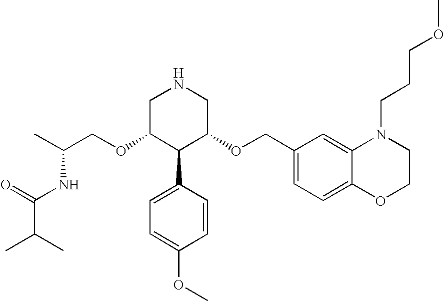 | pale yellow oil | 0.22 (A) | 3.71 (I) |
| 132 | 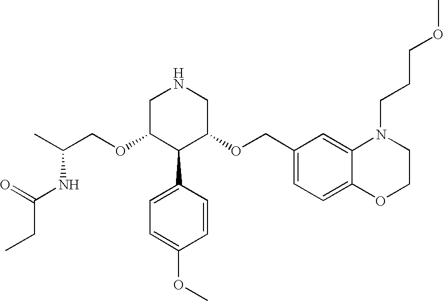 | pale yellow oil | 0.19 (A) | 3.57 (I) |
| 133 | 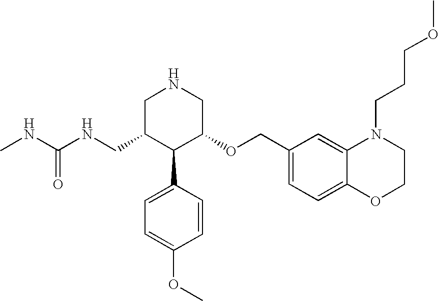 | pale yellow foam | 0.10 (I) | 3.29 (I) |
| 134 | 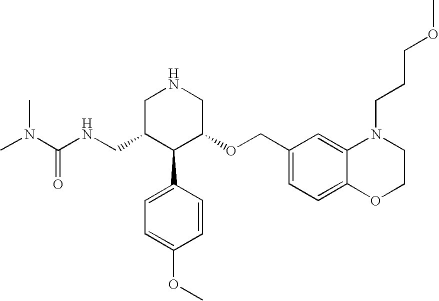 | pale yellow foam | 0.12 (I) | 3.44 (I) |
| 135 | 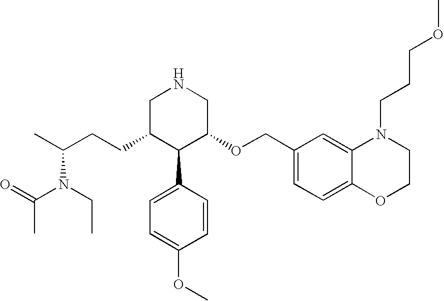 | pale yellow oil | 0.21 (A) | 3.67 (I) |
| 136 | 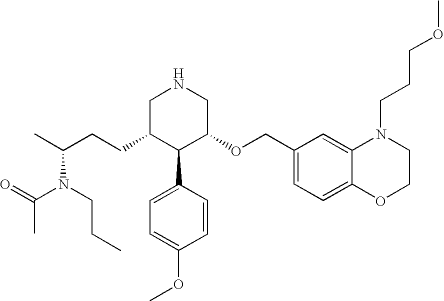 | pale yellow oil | 0.27 (A) | 3.85 (I) |
| 137 | 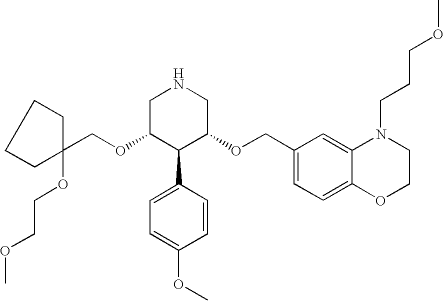 | yellow oil | 0.46 (A) | 4.10 (I) |
| 138 | 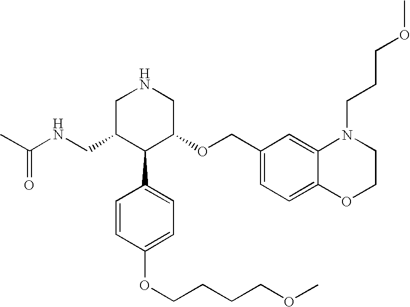 | pale yellow oil | 0.35 (A) | 3.61 (I) |
| 139 | 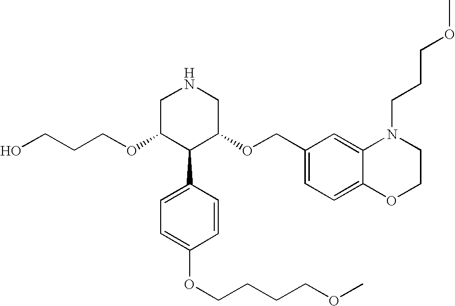 | pink oil | 0.35 (A) | 3.63 (I) |

Mobile phase systems for thin-layer chromatography:
A Dichloromethane-methanol-25% conc. ammonia = 200:20:1
B Dichloromethane-methanol-25% conc. ammonia = 200:20:0.5
C Dichloromethane-methanol-25% conc. ammonia = 200:10:1
D Dichloromethane-methanol-25% conc. ammonia = 90:10:1
E Dichloromethane-methanol-25% conc. ammonia = 60:10:1
F Dichloromethane-methanol-25% conc. ammonia = 200:30:1
G Dichloromethane-methanol = 9:1
H Dichloromethane-methanol-25% conc. ammonia = 200:40:1
I Dichloromethane-methanol-25% conc. ammonia = 100:10:1
J Dichloromethane-methanol = 20:1
K Dichloromethane-methanol-25% conc. ammonia = 40:10:1

HPLC gradient on Hypersil BDS C-18 (5 μm); column: 4×125 mm
(I) 90% water*/10% acetonitrile* to 0% water*/100% acetonitrile* in 5 minutes+2.5 minutes (1.5 ml/min)
(II) 95% water*/5% acetonitrile* to 0% water*/100% acetonitrile* in 40 minutes (0.8 ml/min)

HPLC gradient on Zorbax SB-C18 (3.5 μm); column: 2.1×30 mm
(III) 97.5% water*/2.5% acetonitrile* to 5% water*/95% acetonitrile* in 5.5 minutes+2.4 minutes (0.5 ml/min)
   * contains 0.1% trifluoroacetic acid

The following abbreviations are used:
Rf ratio of distance migrated by a substance to the distance of the solvent front from the starting point in thin-layer chromatography
Rt retention time of a substance in HPLC (in minutes)
M.p. melting point (temperature)

### General Method A: (N-BOC Deprotection)

15 ml of methanol and 2.5 ml of 2N HCl are successively added to a solution of 1 mmol “N-BOC derivative” in 5 ml of chloroform, and the mixture is stirred at 60° C. for 18 hours. The reaction mixture is cooled to room temperature, poured into 1M aqueous sodium bicarbonate solution (40 ml) and extracted with tert-butyl methyl ether (2×60 ml). The organic phases are washed with brine (1×60 ml), dried with sodium sulfate and evaporated. The title compound is obtained from the residue by flash chromatography (SiO₂ 60F).

### General Method B: (Hydrogenation)

A solution of 1 mmol of “substrate” in 15 ml of tetrahydrofuran/methanol 1:1 is hydrogenated in the presence of 100-200 mg of Pd/C 10% at 15-20° C. for 2-20 hours. The reaction mixture is clarified by filtration and the filtrate is evaporated. The title compound is obtained from the residue by flash chromatography (SiO₂ 60F).

### General Method C: (9-BBN Reduction)

A solution of 1 mmol of “lactam” in 3 ml of tetrahydrofuran is mixed with 3.2-6.4 mmol of 9-BBN (0.5M in tetrahydrofuran) and stirred under reflux for 1-2 hours (conversion checked by HPLC). The reaction mixture is cooled to room temperature and, after addition of 3.2-6.4 mmol of ethanolamine, evaporated. The residue is stirred in ethyl acetate/heptane 1:1 (30 ml) at 0° C. overnight and clarified by filtration, and the filtrate is evaporated. The title compound is obtained from the residue by flash chromatography (SiO₂ 60F).

### General Method D: (O-alkylation)

1.1 mmol of sodium hydride (60% dispersion in oil) are added to a solution of 1 mmol of “alcohol”, 1.0-2.0 mmol of “benzyl halide” in 2.0 ml of N,N-dimethylformamide while stirring at −10° C. The reaction mixture is stirred at −10° C. for 1 hour and at room temperature for 18 hours. The mixture is poured into 1 M aqueous sodium bicarbonate solution (50 ml) and extracted with tert-butyl methyl ether (2×50 ml). The organic phases are washed successively with water (1×50 ml) and brine (1×60 ml), dried with sodium sulfate and evaporated. The title compound is obtained from the residue by flash chromatography (SiO₂ 60F).

### General Method E: (Chlorination)

A solution of 40 mmol of “benzyl alcohol” in 6.40 ml of pyridine and 100 ml of dichloro-methane is slowly added dropwise to a solution, precooled to 0-5° C., of 7.65 ml of thionyl chloride in 20 ml of dichloromethane. The reaction mixture is stirred at 0° C. and then at room temperature for 1 hour each, and then poured into 200 ml of ice-water. The mixture is extracted with dichloromethane (2×200 ml). The organic phases are washed successively with 1 M aqueous sodium bicarbonate solution (2×200 ml) and brine, dried with sodium sulfate and evaporated. The title compound is obtained from the residue by flash chromatography (SiO₂ 60F).

### General Method F: (Phenol Alkylation 1)

A mixture of 20 mmol of “phenol” in 60 ml of N,N-dimethylformamide with 4.15 g of potassium carbonate and 30 mmol of “halide” or “tosylate” is stirred at 100° C. for 24 hours. The reaction mixture is then evaporated. The residue is mixed with 1 M aqueous sodium bicarbonate solution (40 ml) and extracted with ethyl acetate (2×60 ml). The organic phases are washed with brine (1×60 ml), dried with sodium sulfate and evaporated. The title compound is obtained from the residue by flash chromatography (SiO₂ 60F).

### General Method G: (Phenol Alkylation II)

A suspension of 1 mmol of “tosylate”, 2 mmol “phenol”, 2 mmol of potassium carbonate and 20 ml of acetonitrile is stirred at 90° C. for 24 h. The reaction mixture is then evaporated. The residue is mixed with saturated aqueous sodium bicarbonate solution and extracted with ethyl acetate (2×). The organic phases are washed with brine, dried with sodium sulfate and evaporated. The title compound is obtained from the residue by flash chromatography (SiO₂ 60F).

### General Method H: (Tosylation)

A solution of 12 mmol of p-toluenesulfonyl chloride in 15 ml of dichloromethane is added dropwise to a solution of 10 mmol of “alcohol”, 15 mmol of triethylamine, 1 mmol of 4-dimethylaminopyridine in 90 ml of dichloromethane at 0° C. The reaction mixture is stirred at room temperature for 2-18 hours. The reaction mixture is diluted with dichloromethane and then washed with water and brine, dried with sodium sulfate and evaporated. The title compound is obtained from the residue by flash chromatography (SiO₂ 60F).

### General Method 1: (Phenol Alkylation III)

A suspension of 1 mmol of “phenol”, 1.0-1.5 mmol of “tosylate” or “bromide”, 1.5 mmol of caesium carbonate and 2 ml of acetonitrile is stirred at 80° C. for 2 hours. The reaction mixture is cooled, poured into water and extracted with ethyl acetate (2×). The organic phases are washed with brine, dried with sodium sulfate and evaporated. The title compound is obtained from the residue by flash chromatography (SiO₂ 60F).

### General Method J (Alcohol Desilylation)

A solution of 1 mmol of “silyl ether” in 5 ml of tetrahydrofuran is mixed with 1.5-2.0 mmol of tetrabutylammonium fluoride (1 M solution in tetrahydrofuran), and the solution is stirred at room temperature for 1-2 hours. The reaction solution is then diluted with water and extracted 2× with tert-butyl methyl ether. The combined organic phases are dried with sodium sulfate and evaporated. The title compound is obtained from the residue by flash chromatography (SiO₂ 60F).

### General Method K (Borane Reduction)

A solution of 1 mmol of “lactam” in 3 ml of tetrahydrofuran is mixed with 3.0-6.0 mmol of borane-tetrahydrofuran complex (1 M in tetrahydrofuran) and stirred at room temperature for 1-3 hours (conversion checked by HPLC or TLC). The reaction mixture is cooled to room temperature, mixed with 3.0-6.0 mmol of methanol and evaporated. The title compound is obtained from the residue by flash chromatography (SiO₂ 60F).

### General Method L (N-Tos Deprotection 1)

0.44 mmol of sodium dihydrogen phosphate and 0.90 mmol of sodium amalgam (10% Na) are successively added at room temperature to a solution of 0.09 mmol of “tosylamide” in 10 ml of methanol. The reaction mixture is stirred for 2-18 hours, diluted with water and extracted with ethyl acetate. The organic phase is separated off and washed with brine, dried with sodium sulfate and evaporated. The title compound is obtained from the residue by flash chromatography (SiO₂ 60F).

### General Method M (O-alkylation II)

1 mmol of methylmagnesium bromide (35% solution in diethyl ether) is added to a solution of 1 mmol of “secondary alcohol” in 5 ml of tetrahydrofuran at room temperature. The reaction solution is heated to reflux for 5 minutes and then a solution of 2.2 mmol of “oxirane” in 1 ml of THF is added. The reaction mixture is heated to reflux for 1-5 hours and poured into saturated aqueous sodium bicarbonate solution, and the mixture is extracted with tert-butyl methyl ether. The combined organic phases are dried over sodium sulfate and evaporated. The title compound is obtained from the residue by flash chromatography (SiO₂ 60F).

### General Method N(N-Tos Deprotection II)

0.5 ml of a bluish green sodium naphthalenide stock solution (from 0.04 g of sodium and 0.22 g of naphthalene in 5 ml of dimethoxyethane) is added to a solution of 0.1 mmol of “tosylamide” in 2 ml of dimethoxyethane at −60° C. After 3-6 hours, the reaction mixture is diluted with water and extracted with dichloromethane (2×). The combined organic phases are washed with brine, dried with sodium sulfate and evaporated. The title compound is obtained from the residue by flash chromatography (SiO₂ 60F).

### Example 1

### {(3S,4R,5R)-4-(4-Methoxyphenyl)-5-[4-(3-methoxypropyl)-3,4-dihydro-2H-benzo[1,4]oxazin-6-ylmethoxy]piperidin-3-yloxyl}acetonitrile

The title compound is prepared in analogy to method L from 0.185 g of [(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]acetonitrile.

The starting materials are prepared as follows:

### a) [(3S,4R,5R)-4-(4-Methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]acetonitrile

0.164 g of sodium hydride (60% dispersion in oil) is added to a stirred solution of 0.50 g of (3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-ol in 4 ml of acetonitrile. The reaction mixture is stirred at room temperature for 1 hour. 0.762 g of bromoacetonitrile is added at −20° C., and the mixture is stirred at −20° C. for 48 hours. The reaction mixture is poured into 1 M aqueous sodium bicarbonate solution (30 ml) and extracted with tert-butyl methyl ether (2×100 ml). The organic phases are dried with sodium sulfate and evaporated. The title compound is obtained as a yellowish resin from the residue by flash chromatography (SiO₂ 60F). Rf=0.30 (EtOAc-heptane 1:1); Rt=5.13 (Gradient I).

### b) (3S,4S,5R)-4-(4-Methoxyphenyl)-5-[4-(3-methoxypropyl)-3,4-dihydro-2H-benzo[1,4]oxazin-6-ylmethoxy]-1-(toluene-4-sulfonyl)piperidin-3-ol

14.74 g of tetrabutylammonium fluoride trihydrate are added to a solution of 23.2 g of 6-[(3R,4R,5S)-4-(4-methoxyphenyl)-1-(toluene-4-sulfonyl)-5-triisopropylsilanyloxypiperidin-3-yloxymethyl]-4-(3-methoxypropyl)-3,4-dihydro-2H-benzo[1,4]oxazine in 500 ml of tetrahydro-furan at room temperature. After 1 hour 10 ml of water are added to the reaction mixture, and the mixture is evaporated to dryness. The title compound is obtained as a yellowish oil from the residue by flash chromatography (SiO₂ 60F). Rf=0.06 (EtOAc-heptane 1:2); Rt=4.73 (Gradient I).

### c) 6-[(3R,4R,5S)-4-(4-Methoxyphenyl)-1-(toluene-4-sulfonyl)-5-triisopropylsilanyloxy-piperidin-3-yloxymethyl]-4-(3-methoxypropyl)-3,4-dihydro-2H-benzo[1,4]oxazine

24.99 g of 6-[(3R,4R,5S)-4-(4-methoxyphenyl)-1-(toluene-4-sulfonyl)-5-triisopropylsilanyloxy-piperidin-3-yloxymethyl]-4-(3-methoxypropyl)-4H-benzo[1,4]oxazin-3-one are reacted in analogy to method C. The title compound is obtained as a colourless resin. Rf=0.10 (EtOAc-heptane 1:2).

### d) 6-[(3R,4R,5S)-4-(4-Methoxyphenyl)-1-(toluene-4-sulfonyl)-5-triisopropylsilanyloxy-piperidin-3-yloxymethyl]-4-(3-methoxypropyl)-4H-benzo[1,4]oxazin-3-one

19.0 g of (3R,4R,5S)-4-(4-methoxyphenyl)-1-(toluene-4-sulfonyl)-5-triisopropylsilanyloxy-piperidin-3-ol and 11.52 g of 6-chloromethyl-4-(3-methoxypropyl)-4H-benzo[1,4]oxazin-3-one are reacted in analogy to method D. The title compound is obtained as a yellowish resin. Rf=0.18 (EtOAc-heptane 1:2); Rt=6.67 (Gradient I).

### e) (3R,4R,5S)-4-(4-Methoxyphenyl)-1-(toluene-4-sulfonyl)-5-triisopropylsilanyloxypiperidin-3-ol

A suspension of 55.64 g (3R,4R,5S)-4-(4-hydroxyphenyl)-1-(toluene-4-sulfonyl)-5-triisopropylsilanyloxypiperidin-3-ol, 15.0 g of dimethyl sulfate, 20.92 g of potassium carbonate and 750 ml of acetone is stirred at 80° C. for 24 h. The reaction mixture is clarified by filtration and evaporated. The residue is diluted with 1.75 l of tert-butyl methyl ether, and 1 l of water is added. The aqueous phase is again extracted with 1 l of tert-butyl methyl ether. The combined organic phases are washed with 750 ml of brine, dried over sodium sulfate and evaporated. The crude title compound is obtained as a white foam from the residue. Rt=6.27 (Gradient I).

### f) (3R,4R,5S)-4-(4-Hydroxyphenyl)-1-(toluene-4-sulfonyl)-5-triisopropylsilanyloxypiperidin-3-ol

26.08 g of toluenesulfonyl chloride are added to a mixture of 50 g of (3R,4R,5S)-4-(4-hydroxyphenyl)-5-triisopropylsilanyloxypiperidin-3-ol in 1 l of ethyl acetate and 1 l of 2N sodium carbonate solution at 0° C. After 4 hours at 0° C., the reaction mixture is stirred at room temperature for a further 16 hours. The phases are separated and the aqueous phase is extracted with 200 ml of ethyl acetate. The combined organic phases are washed with 200 ml of brine, dried with sodium sulfate and evaporated. The title compound is obtained in the form of white crystals. Rf=0.31 (EtOAc-heptane 1:1.5); Rt=5.77 (Gradient I).

### g) (3R,4R,5S)-4-(4-Hydroxyphenyl)-5-triisopropylsilanyloxypiperidin-3-ol

5.210 g of (3R,4R,5S)-4-(4-benzyloxyphenyl)-1-((R)-1-phenylethyl)-5-triisopropylsilanyloxy-piperidin-3-ol are reacted in analogy to method B. The title compound is obtained as a colourless solid. Rf=0.19 (dichloromethane-methanol-25% conc. ammonia=200:20:1); Rt=3.80 (Gradient I).

### h) (3R,4R,5S)-4-(4-Benzyloxyphenyl)-1-((R)-1-phenylethyl)-5-triisopropylsilanyloxy-piperidin-3-ol

150 ml of borane-tetrahydrofuran complex (1M in tetrahydrofuran) are added dropwise to a solution of 20.00 g of (S)-4-(4-benzyloxyphenyl)-1-((R)-1-phenylethyl)-3-triisopropyl-silanyloxy-1,2,3,4-tetrahydropyridine in 280 ml of 1,2-dimethoxyethane at 0° C. The reaction solution is then stirred at 30° C. for 3 hours. The solution is cooled to room temperature and quenched with 70 ml of water. After stirring for 5 minutes, 56.00 g of sodium percarbonate are added, and the suspension is stirred at 50° C. for 1 hour. The reaction mixture is poured into 600 ml of water and extracted with ethyl acetate (2×). The combined organic phases are washed with 400 ml each of water and brine and evaporated. The title compound is obtained as a yellowish oil from the residue by flash chromatography (SiO₂ F60). Rf=0.23 (EtOAc-heptane 1:2); Rt=5.75 (Gradient I).

### i) (S)-4-(4-Benzyloxyphenyl)-1-((R)-1-phenylethyl)-3-triisopropylsilanyloxy-1,2,3,6-tetrahydropyridine

6.80 ml of 2,6-lutidine are added to a suspension of 14.70 g of 4-(4-benzyloxyphenyl)-1-(1(R)-phenylethyl)-1,2,3,6-tetrahydropyridin-3(S)-ol [257928-45-3] in 250 ml of dichloro-methane, and the mixture is cooled to 0° C. 12.60 ml of triisopropysilyl trifluoromethane-sulfonate are added dropwise, and the reaction mixture is stirred at 0° C. for 1 hour. The reaction solution is poured into 400 ml of water and the phases are separated. The aqueous phase is back-extracted with 200 ml of dichloromethane, and the combined organic phases are dried with sodium sulfate and evaporated. The title compound is obtained as a yellow-brown oil from the residue by flash chromatography (SiO₂ F60). Rf=0.66 (EtOAc-heptane 1:2); Rt=5.83 (Gradient I).

### j) 6-Chloromethyl-4-(3-methoxypropyl)-4H-benzo[1,4]oxazin-3-one

0.37 g of 6-hydroxymethyl-4-(3-methoxypropyl)-4H-benzo[1,4]oxazin-3-one is reacted in analogy to method E. The title compound is obtained as a colourless oil. Rf=0.60 (EtOAc-heptane 2:1); Rt=4.05 (Gradient I).

### k) 6-Hydroxymethyl-4-(3-methoxypropyl)-4H-benzo[1,4]oxazin-3-one

A suspension of 1.79 g of 6-hydroxymethyl-4H-benzo[1,4]oxazin-3-one, 2.20 ml of 1-chloro-3-methoxypropane, 10 g of potassium fluoride on alumina and 0.033 g of potassium iodide in 150 ml of acetonitrile is stirred under reflux for 72 hours. The reaction mixture is cooled and clarified by filtration, and the filtrate is evaporated to dryness. The title compound is obtained as a yellow oil from the residue by flash chromatography (SiO₂ 60F). Rf=0.60 (dichloro-methane-methanol 9:1); Rt=2.74 (Gradient I).

### m) 6-Hydroxymethyl-4H-benzo[1,4]oxazin-3-one

A mixture of 6.9 g of methyl 3-oxo-3,4-dihydro-2H-benzo[1,4]oxazine-6-carboxylate [202195-67-3] and 230 ml of tetrahydrofuran is cooled to −40° C. 88.9 ml of diisobutylaluminium hydride (1.5M in toluene) are added dropwise at −40° C. over the course of 30 minutes. The reaction mixture is stirred at −40° C. to −20° C. for 1.5 hours and then cautiously poured into 150 ml of 2N HCl (cold). The organic phase is separated off and the aqueous phase is extracted with tetrahydrofuran (5×100 ml). The organic phases are washed with brine (1×100 ml), filtered through cotton wool and evaporated. The title compound is obtained as beige crystals from the residue by crystallization (from ethanol). Rf=0.16 (EtOAc-heptane 2:1); Rt=2.23 (Gradient I); m.p.: 186-187° C.

### Example 2

### (R)-1-Methoxy-3-{(3S,4R,5R)-4-(4-methoxyphenyl)-5-[4-(3-methoxypropyl)-3,4-dihydro-2H-benzo[1,4]oxazin-6-ylmethoxy]piperidin-3-yloxy}propan-2-ol

The title compound is prepared from 0.282 g of (R)-1-methoxy-3-[(3S,4R,5R)-4-(4-methoxy-phenyl)-5-[4-(3-methoxypropyl)-3,4-dihydro-2H-benzo[1,4]oxazin-6-ylmethoxy]-1-(toluene-4-sulfonyl)piperidin-3-yloxy]-propan-2-ol in analogy to method L.

The starting material is prepared as follows:

### a) (R)-1-Methoxy-3-[(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]propan-2-ol

0.30 g of (3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-ol (Example 1b) and 0.098 g of S-(+)-glycidyl methyl ether [64491-68-5] are reacted in analogy to method M. The title compound is obtained as a colourless oil. Rf=0.19 (EtOAc-heptane 2:1); Rt=4.81 (Gradient I).

The following compound is prepared in an analogous manner to the process described in Example 2:

### Example 3

### (S)-1-Methoxy-3-{(3S,4R,5R)-4-(4-methoxyphenyl)-5-[4-(3-methoxypropyl)-3,4-dihydro-2H-benzo[1,4]oxazin-6-ylmethoxy]piperidin-3-yloxy}propan-2-ol

using R-(−)-glycidyl methyl ether [64491-70-9]

### Example 5

### 6-[(3R,4R,5S)-4-(4-Methoxyphenyl)-5-prop-2-ynyloxypiperidin-3-yloxymethyl]-4-(3-methoxy-propyl)-3,4-dihydro-2H-benzo[1,4]oxazine

0.236 g of sodium dihydrogenphosphate and 0.743 g of sodium amalgan (10% Na) are successively added to a solution of 0.209 g of 6-[(3R,4R,5S)-4-(4-methoxyphenyl)-5-prop-2-ynyloxy-1-(toluene-4-sulfonyl)piperidin-3-yloxymethyl]-4-(3-methoxypropyl)-3,4-dihydro-2H-benzo[1,4]oxazine in 20 ml of tetrahydrofuran at 0° C. The reaction mixture is stirred at 0° C. for 1 hour and then at room temperature for 20 hours. The mixture is decanted and clarified by filtration through Hyflo. The filtrate is evaporated. The title compound is obtained from the residue by flash chromatography (SiO₂ 60F).

The starting materials are prepared as follows:

### a) 6-[(3R,4R,5S)-4-(4-Methoxyphenyl)-5-prop-2-ynyloxy-1-(toluene-4-sulfonyl)piperidin-3-yloxymethyl]-4-(3-methoxypropyl)-3,4-dihydro-2H-benzo[1,4]oxazine

0.263 g of sodium hydride (60% dispersion in oil) is added to a solution of 1.50 g of (3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-ol (Example 1b) and 1.099 g of 3-bromo propyne in 25 ml of tetrahydrofuran. After stirring for 22 hours, the reaction mixture is mixed with 40 ml of saturated sodium bicarbonate solution and extracted with tert-butyl methyl ether (3×50 ml). The combined organic phases are washed with 40 ml of brine, dried with sodium sulfate and evaporated. The title compound is obtained as a yellowish resin from the residue by flash chromatography (SiO₂ 60F). Rf=0.18 (EtOAc-heptane 1:2); Rt=5.26 (Gradient I).

### Example 6

### 6-[(3R,4R,5S)-4-(4-Methoxyphenyl)-5-(4-morpholin-4-yl-but-2-ynyloxy)piperidin-3-yloxy-methyl]-4-(3-methoxypropyl)-3,4-dihydro-2H-benzo[1,4]oxazine

0.194 g of sodium dihydrogenphosphate and 0.612 g of sodium amalgan (10% Na) are successively added to a solution of 0.199 g of 6-[(3R,4R,5S)-4-(4-methoxyphenyl)-5-(4-morpholin-4-ylbut-2-ynyloxy)-1-(toluene-4-sulfonyl)piperidin-3-yloxymethyl]-4-(3-methoxy-propyl)-3,4-dihydro-2H-benzo[1,4]oxazine in 20 ml of tetrahydrofuran at 0° C. The reaction mixture is stirred at 0° C. for 1 hour and then at room temperature for 24 hours. The mixture is decanted and clarified by filtration through Hyflo. The filtrate is evaporated. The title compound is obtained from the residue by flash chromatography (SiO₂ 60F).

The starting materials are prepared as follows:

### a) 6-[(3R,4R,5S)-4-(4-Methoxyphenyl)-5-(4-morpholin-4-ylbut-2-ynyloxy)-1-(toluene-4-sulfonyl)piperidin-3-yloxymethyl]-4-(3-methoxypropyl)-3,4-dihydro-2H-benzo[1,4]oxazine

A suspension of 0.038 g of paraformaldehyde and 0.032 g of morpholine in 4 ml of dioxane is heated until the solution is clear and then stirred at room temperature for 20 minutes. A solution of 0.21 g of 6-[(3R,4R,5S)-4-(4-methoxyphenyl)-5-prop-2-ynyloxy-1-(toluene-4-sulfonyl)piperidin-3-yloxymethyl]-4-(3-methoxypropyl)-3,4-dihydro-2H-benzo[1,4]oxazine (Example 5a) and 0.068 g of copper(II) acetate in 1 ml of dioxane is added to this solution. After 18 hours at 90° C., the reaction mixture is diluted with tert-butyl methyl ether and washed successively with water and brine. The organic phase is dried with sodium sulfate and evaporated. The title compound is obtained as a yellowish resin from the residue by flash chromatography (SiO₂60F). Rf=0.19 (EtOAc-heptane 2:1); Rt=4.55 (Gradient I).

The following compound is prepared in an analogous manner to the process described in Example 6:

### Example 7

### (4-{(3S,4R,5R)-4-(4-Methoxyphenyl)-5-[4-(3-methoxypropyl)-3,4-dihydro-2H-benzo[1,4]oxazin-6-ylmethoxy]piperidin-3-yloxy}-but-2-ynyl)dimethylamine

### Example 8

### 6-[(3R,4R,5S)-5-(2-Methoxy-2-methylpropoxy)-4-(4-methoxyphenyl)piperidin-3-yloxymethyl]-4-(3-methoxypropyl)-3,4-dihydro-2H-benzo[1,4]oxazine

The title compound is prepared from 0.20 g of 6-[(3R,4R,5S)-5-(2-methoxy-2-methyl-propoxy)-4-(4-methoxyphenyl)-1-(toluene-4-sulfonyl)piperidin-3-yloxymethyl]-4-(3-methoxy-propyl)-3,4-dihydro-2H-benzo[1,4]oxazine in analogy to method L.

The starting materials are prepared as follows:

### a) 6-[(3R,4R,5S)-5-(2-Methoxy-2-methylpropoxy)-4-(4-methoxyphenyl)-1-(toluene-4-sulfonyl)piperidin-3-yloxymethyl]-4-(3-methoxypropyl)-3,4-dihydro-2H-benzo[1,4]oxazine

0.03 g of sodium hydride (60% dispersion in oil) is added to a stirred solution of 0.20 g of 1-[(3S,4R,5R)-4-(4-methoxyphenyl)-5-[4-(3-methoxypropyl)-3,4-dihydro-2H-benzo[1,4]-oxazin-6-ylmethoxy]-1-(toluene-4-sulfonyl)piperidin-3-yloxy]-2-methylpropan-2-ol in 1.5 ml of N,N-dimethylformamide. After 45 minutes at room temperature, 0.129 g of methyl iodide is added to the mixture. After 3 hours at room temperature, the reaction mixture is diluted with tert-butyl methyl ether. The solution is washed successively with aqueous sodium bicarbonate solution, water and brine, dried with sodium sulfate and evaporated. The title compound is obtained as a yellowish oil from the residue by flash chromatography (SiO₂60F). Rt=5.56 (Gradient I).

### b) 1-[(3S,4R,5R)-4-(4-Methoxyphenyl)-5-[4-(3-methoxypropyl)-3,4-dihydro-2H-benzo[1,4]-oxazin-6-ylmethoxy]-1-(toluene-4-sulfonyl)piperidin-3-yloxy]-2-methylpropan-2-ol

0.558 ml of a solution of methylmagnesium bromide (3N in diethyl ether) is added to a solution of 0.23 g of methyl [(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]acetate in 1.5 ml of tetrahydrofuran. After 15 minutes at 50° C., the reaction mixture is cooled to room temperature and diluted with 50 ml of tert-butyl methyl ether. The solution is washed successively with 20 ml of saturated aqueous sodium bicarbonate solution and 10 ml of brine. The combined aqueous phases are extracted with 50 ml of tert-butyl methyl ether. The combined organic phases are dried with sodium sulfate and evaporated. The title compound is obtained as a white resin from the residue. Rt=5.16 (Gradient I).

### c) Methyl [(3S,4R,5R)-4-(4-Methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]acetate

0.085 g of sodium hydride (60% dispersion in oil) is added to a solution of 0.513 g of (3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-ol (Example 1b) in 8 ml of tetrahydrofuran while stirring. The reaction mixture is stirred at room temperature for 1 hour and then, at −20° C., 0.40 g of methyl bromoacetate is added. After 1 hour at −20° C. and 3 hours at room temperature, the reaction mixture is diluted with 100 ml of tert-butyl methyl ether and washed with saturated aqueous sodium bicarbonate solution (30 ml). The aqueous phase is extracted with tert-butyl methyl ether (2×50 ml). The combined organic phases are dried with sodium sulfate and evaporated. The title compound is obtained as a yellowish resin from the residue by flash chromatography (SiO₂ 60F). Rf=0.14 (EtOAc-heptane 1:2); Rt=5.21 (Gradient I).

### Example 9

### 1-{(3S,4R,5R)-4-(4-Methoxyphenyl)-5-[4-(3-methoxypropyl)-3,4-dihydro-2H-benzo[1,4]-oxazin-6-ylmethoxy]piperidin-3-yloxyl}-2-methylpropan-2-ol

The title compound is prepared from 0.125 g of 1-[(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)-piperidin-3-yloxy]-2-methylpropan-2-ol (Example 8b) in analogy to method L.

The following compound is prepared in an analogous manner to the process described in Example 9:

### Example 10

### 3-{(3S,4R,5R)-4-(4-Methoxyphenyl)-5-[4-(3-methoxypropyl)-3,4-dihydro-2H-benzo[1,4]oxazin-6-ylmethoxy]piperidin-3-yloxymethyl}pentan-3-ol

### Example 11

### (R)-1-Methoxy-3-[(3S,4R,5R)-5-[4-(3-methoxypropyl)-3,4-dihydro-2H-benzo[1,4]oxazin-6-yl-methoxy]-4-(4-propoxyphenyl)piperidin-3-yloxy]propan-2-ol

The title compound is prepared from 0.303 g of benzyl (3S,4R,5R)-4-(4-allyloxyphenyl)-3-((R)-2-hydroxy-3-methoxypropoxy)-5-[4-(3-methoxypropyl)-3,4-dihydro-2H-benzo[1,4]oxazin-6-ylmethoxy]piperidine-1-carboxylate in analogy to method B.

The starting materials are prepared as follows:

### a) Benzyl (3S,4R,5R)-4-(4-allyloxyphenyl)-3-((R)-2-hydroxy-3-methoxypropoxy)-5-[4-(3-methoxypropyl)-3,4-dihydro-2H-benzo[1,4]oxazin-6-ylmethoxy]piperidin-1-carboxylate

The title compound is obtained as a colourless oil from 1.400 g of benzyl (3S,4S,5R)-4-(4-allyloxyphenyl)-3-hydroxy-5-[4-(3-methoxypropyl)-3,4-dihydro-2H-benzo[1,4]oxazin-6-yl-methoxy]piperidine-1-carboxylate and 0.460 g of S-(+)-glycidyl methyl ether [64491-68-5] in analogy to method M. Rf=0.34 (EtOAc-heptane 3:1); Rt=5.06 (Gradient I).

### b) Benzyl (3S,4S,5R)-4-(4-allyloxyphenyl)-3-hydroxy-5-[4-(3-methoxypropyl)-3,4-dihydro-2H-benzo[1,4]oxazin-6-ylmethoxy]piperidin-1-carboxylate

The title compound is obtained as a colourless resin from 1.860 g of benzyl (3R,4R,5S)-4-(4-allyloxyphenyl)-3-[4-(3-methoxypropyl)-3,4-dihydro-2H-benzo[1,4]oxazin-6-ylmethoxy]-5-tri-isopropylsilanyloxypiperidine-1-carboxylate in analogy to method J. Rf=0.18 (EtOAc-heptane 1:1); Rt=4.97 (Gradient I).

### c) Benzyl (3R,4R,5S)-4-(4-allyloxyphenyl)-3-[4-(3-methoxypropyl)-3,4-dihydro-2H-benzo-[1,4]oxazin-6-ylmethoxy]-5-triisopropylsilanyloxypiperidine-1-carboxylate

0.820 g of potassium carbonate and 0.300 ml of allyl bromide are added to a solution of 2.090 g of benzyl (3R,4R,5S)-4-(4-hydroxyphenyl)-3-[4-(3-methoxypropyl)-3,4-dihydro-2H-benzo[1,4]oxazin-6-ylmethoxy]-5-triisopropylsilanyloxypiperidine-1-carboxylate in 5 ml of N,N-dimethylformamide at room temperature. The reaction mixture is stirred at 40-60° C. for 5 hours, poured into saturated aqueous sodium bicarbonate solution and extracted with tert-butyl methyl ether. The combined organic extracts are washed with brine, dried with sodium sulfate and evaporated. The title compound is obtained as a colourless oil from the residue by flash chromatography (SiO₂ 60F). Rf=0.42 (EtOAc-heptane 1:1).

### d) Benzyl (3R,4R,5S)-4-(4-hydroxyphenyl)-3-[4-(3-methoxypropyl)-3,4-dihydro-2H-benzo-[1,4]oxazin-6-ylmethoxy]-5-triisopropylsilanyloxypiperidine-1-carboxylate

The title compound is obtained as a reddish resin from 5.010 g of benzyl (3R,4R,5S)-4-(4-hydroxyphenyl)-3-[4-(3-methoxypropyl)-3-oxo-3,4-dihydro-2H-benzo[1,4]oxazin-6-yl-methoxy]-5-triisopropylsilanyloxypiperidine-1-carboxylate in analogy to method K. Rf=0.32 (EtOAc-heptane 1:1); Rt=29.32 (Gradient II).

### e) Benzyl (3R,4R,5S)-4-(4-hydroxyphenyl)-3-[4-(3-methoxypropyl)-3-oxo-3,4-dihydro-2H-benzo[1,4]oxazin-6-ylmethoxy]-5-triisopropylsilanyloxypiperidine-1-carboxylate

1.620 g of palladium(0) tetrakistriphenylphosphine and 7.300 g of potassium carbonate are added to a solution of 13.88 g of benzyl (3R,4R,5S)-4-(4-allyloxyphenyl)-3-[4-(3-methoxy-propyl)-3-oxo-3,4-dihydro-2H-benzo[1,4]oxazin-6-ylmethoxy]-5-triisopropylsilanyloxy-piperidine-1-carboxylate in 100 ml of methanol. The reaction mixture is then stirred at room temperature for 3 hours. The solid is filtered off and the filtrate is evaporated. The title compound is obtained as a colourless oil from the residue by flash chromatography (SiO₂ 60F). Rf=0.23 (EtOAc-heptane 1:1); Rt=6.37 (Gradient I).

### f) Benzyl (3R,4R,5S)-4-(4-allyloxyphenyl)-3-[4-(3-methoxypropyl)-3-oxo-3,4-dihydro-2H-benzo[1,4]oxazin-6-ylmethoxy]-5-triisopropylsilanyloxypiperidine-1-carboxylate

The title compound is obtained as a yellow resin from 16.50 g of benzyl (3R,4R,5S)-4-(4-allyloxyphenyl)-3-hydroxy-5-triisopropylsilanyloxypiperidine-1-carboxylate in analogy to method D. Rf=0.18 (EtOAc-heptane 1:2); Rt=7.07 (Gradient I).

### g) Benzyl (3R,4R,5S)-4-(4-allyloxyphenyl)-3-hydroxy-5-triisopropylsilanyloxypiperidine-1-carboxylate

16.66 g of benzyl (3R,4R,5S)-3-hydroxy-4-(4-hydroxyphenyl)-5-triisopropylsilanyloxy-piperidine-1-carboxylate are reacted in analogy to method 11c. The title compound is obtained as a pale brown resin. Rf=0.42 (EtOAc-heptane 1:1); Rt=6.54 (Gradient I).

### h) Benzyl (3R,4R,5S)-3-hydroxy-4-(4-hydroxyphenyl)-5-triisopropylsilanyloxypiperidine-1-carboxylate

20.07 ml of benzyl chloroformate are slowly added to a two-phase mixture of 50 g of (3R,4R,5S)-4-(4-hydroxyphenyl)-5-triisopropylsilanyloxypiperidin-3-ol (Example 1 g) in 1000 ml of saturated sodium bicarbonate solution and 1000 ml of ethyl acetate at 0° C. After 3 hours at 0° C. and 15 hours at room temperature, the aqueous phase is separated and extracted with 200 ml of ethyl acetate. The combined organic phases are washed with 200 ml of brine, dried with sodium sulfate and evaporated. The title compound is obtained as a colourless oil from the residue by flash chromatography (SiO₂ 60F). Rf=0.31 (EtOAc-heptane 1:1.5); Rt=5.77 (Gradient I).

### Example 12

### (R)-1-3-{(3S,4R,5R)-4-(4-Allyloxyphenyl)-5-[4-(3-methoxypropyl)-3,4-dihydro-2H-benzo[1,4]-oxazin-6-ylmethoxy]piperidin-3-yloxy}-3-methoxypropan-2-ol

5 ml of a 40% aqueous potassium hydroxide solution are added to a solution of 0.375 g of benzyl (3S,4R,5R)-4-(4-allyloxyphenyl)-3-((R)-2-hydroxy-3-methoxypropoxy)-5-[4-(3-methoxypropyl)-3,4-dihydro-2H-benzo[1,4]oxazin-6-ylmethoxy]piperidine-1-carboxylate (Example 11a) in 10 ml of 1:1 methanol-dioxane. The mixture is heated in a closed flask at 80° C. for 5 hours. The reaction solution is poured into water and extracted with tert-butyl methyl ether. The combined organic extracts are washed with brine, dried with sodium sulfate and evaporated. The title compound is obtained from the residue by flash chromatography (SiO₂ 60F).

### Example 13

### (R)-1-[(3S,4R,5R)-4-(4-Ethoxyphenyl)-5-[4-(3-methoxypropyl)-3,4-dihydro-2H-benzo[1,4]-oxazin-6-ylmethoxy]piperidin-3-yloxy]-3-methoxypropan-2-ol

The title compound is prepared from 0.600 g of benzyl (3S,4R,5R)-4-(4-ethoxyphenyl)-3-((R)-2-hydroxy-3-methoxypropoxy)-5-[4-(3-methoxypropyl)-3,4-dihydro-2H-benzo[1,4]oxazin-6-ylmethoxy]piperidine-1-carboxylate in analogy to method B.

The starting materials are prepared as follows:

### a) Benzyl (3S,4R,5R)-4-(4-ethoxyphenyl)-3-((R)-2-hydroxy-3-methoxypropoxy)-5-[4-(3-methoxypropyl)-3,4-dihydro-2H-benzo[1,4]oxazin-6-ylmethoxy]piperidine-1-carboxylate

The title compound is obtained as a yellowish resin from 0.800 g of benzyl (3S,4S,5R)-4-(4-ethoxyphenyl)-3-hydroxy-5-[4-(3-methoxypropyl)-3,4-dihydro-2H-benzo[1,4]oxazin-6-ylmethoxy]piperidine-1-carboxylate and 0.268 g of S-(+)-glycidyl methyl ether [64491-68-5] in analogy to method M. Rf=0.24 (EtOAc-heptane 3:1); Rt=4.98 (Gradient I).

### b) Benzyl (3S,4S,5R)-4-(4-ethoxyphenyl)-3-hydroxy-5-[4-(3-methoxypropyl)-3,4-dihydro-2H-benzo[1,4]oxazin-6-ylmethoxy]piperidine-1-carboxylate

The title compound is obtained as a yellow resin from 2.100 g of benzyl (3R,4R,5S)-4-(4-ethoxyphenyl)-3-[4-(3-methoxypropyl)-3,4-dihydro-2H-benzo[1,4]oxazin-6-ylmethoxy]-5-tri-isopropylsilanyloxypiperidine-1-carboxylate in analogy to method J. Rf=0.27 (EtOAc-heptane 2:1); Rt=4.88 (Gradient I).

### c) Benzyl (3R,4R,5S)-4-(4-ethoxyphenyl)-3-[4-(3-methoxypropyl)-3,4-dihydro-2H-benzo-[1,4]oxazin-6-ylmethoxy]-5-triisopropylsilanyloxypiperidine-1-carboxylate

0.185 g of sodium hydride (60% dispersion in oil) is added to a solution of 2.500 g of benzyl (3R,4R,5S)-4-(4-hydroxyphenyl)-3-[4-(3-methoxypropyl)-3,4-dihydro-2H-benzo[1,4]oxazin-6-ylmethoxy]-5-triisopropylsilanyloxypiperidine-1-carboxylate (Example 11d) and 0.56 ml of ethyl iodide in 35 ml of N,N-dimethylformamide at 0° C. The reaction mixture is stirred at room temperature for 3 hours, poured into saturated aqueous sodium bicarbonate solution and extracted with tert-butyl methyl ether. The combined organic extracts are washed with brine, dried with sodium sulfate and evaporated. The title compound is obtained as a yellow resin from the residue by flash chromatography (SiO₂ 60F). Rf=0.23 (EtOAc-heptane 1:2).

### Example 14

### 4-{(3S,4S,5R)-4-(4-Methoxyphenyl)-5-[4-(3-methoxypropyl)-3,4-dihydro-2H-benzo[1,4]-oxazin-6-ylmethoxy]piperidin-3-yloxy}-2-methylbutan-2-ol

The title compound is prepared from 0.044 g of 4-[(3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)-piperidin-3-yloxy]-2-methylbutan-2-ol in analogy to method L.

The starting materials are prepared as follows:

### a) 4-[(3S,4S,5R)-4-(4-Methoxyphenyl)-5-[4-(3-methoxypropyl)-3,4-dihydro-2H-benzo[1,4]-oxazin-6-ylmethoxy]-1-(toluene-4-sulfonyl)piperidin-3-yloxy]-2-methylbutan-2-ol

0.046 g of methyl 3-[(3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]propionate and 0.109 ml of methylmagnesium bromide solution (3N in diethyl ether) are reacted in analogy to Example 8b. The title compound is obtained as a colourless resin. Rt=5.11 (Gradient I).

### b) Methyl 3-[(3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]propionate

0.043 g of methyl acrylate is added to a solution of 0.10 g of (3S,4S,5R)-4-(4-methoxy-phenyl)-5-[4-(3-methoxypropyl)-3,4-dihydro-2H-benzo[1,4]oxazin-6-ylmethoxy]-1-(toluene-4-sulfonyl)piperidin-3-ol (Example 1b) and 0.013 g of 2,3,4,6,7,8,9,10-octahydropyrimido-[1,2-a]azepine (DBU) in 0.5 ml of acetonitrile. After 18 hours at 45° C., 0.043 g of methyl acrylate is again added to the reaction solution. After 24 hours, the reaction mixture is evaporated. The title compound is obtained as a yellowish oil from the residue by flash chromatography (SiO₂ 60F). Rf=0.47 (EtOAc-heptane 2:1); Rt=5.23 (Gradient I).

### Example 16

### 3-{(3S,4S,5R)-4-(4-Methoxyphenyl)-5-[4-(3-methoxypropyl)-3,4-dihydro-2H-benzo[1,4]-oxazin-6-ylmethoxy]piperidin-3-yloxy}propan-1-ol

The title compound is prepared from 0.088 g of 3-[(3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)-piperidin-3-yloxy]propan-1-ol in analogy to method L.

The starting materials are prepared as follows:

### a) 3-[(3S,4S,5R)-4-(4-Methoxyphenyl)-5-[4-(3-methoxypropyl)-3,4-dihydro-2H-benzo[1,4]-oxazin-6-ylmethoxy]-1-(toluene-4-sulfonyl)piperidin-3-yloxy]propan-1-ol

0.389 g of 6-[(3R,4S,5S)-4-(4-methoxyphenyl)-1-(toluene-4-sulfonyl)-5-(3-triisopropylsilanyl-oxypropoxy)piperidin-3-yloxymethyl]-4-(3-methoxypropyl)-3,4-dihydro-2H-benzo[1,4]oxazine is reacted in analogy to method J. The title compound is obtained as a colourless resin. Rf=0.36 (EtOAc-heptane 4:1); Rt=4.80 (Gradient I).

### b) 6-[(3R,4S,5S)-4-(4-Methoxyphenyl)-1-(toluene-4-sulfonyl)-5-(3-triisopropylsilanyloxy-propoxy)piperidin-3-yloxymethyl]-4-(3-methoxypropyl)-3,4-dihydro-2H-benzo[1,4]oxazine

0.043 g of sodium hydride (60% dispersion in oil) is added to a solution of 0.50 g of (3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-ol (Example 1b), 0.268 g of (3-bromopropoxy)-triisopropylsilane [215650-24-1] and 0.009 g of tetrabutylammonium iodide in 8 ml of tetra-hydrofuran. After 15 hours at 50° C., the reaction mixture is diluted at room temperature with 200 ml of tert-butyl methyl ether. The solution is washed successively with 30 ml of aqueous sodium bicarbonate solution, 30 ml of water and 20 ml of brine, dried with sodium sulfate and evaporated. The title compound is obtained as a colourless oil from the residue by flash chromatography (SiO₂ 60F). Rf=0.31 (EtOAc-heptane 1:2).

### Example 17

### 6-[(3R,4S,5S)-4-(4-Methoxyphenyl)-5-(3-[1,2,4]-triazol-1-yl-propoxy)piperidin-3-yloxymethyl]-4-(3-methoxypropyl)-3,4-dihydro-2H-benzo[1,4]oxazine

The title compound is prepared from 0.150 g of 6-[(3R,4S,5S)-4-(4-methoxyphenyl)-1-(toluene-4-sulfonyl)-5-(3-[1,2,4]triazol-1-yl-propoxy)piperidin-3-yloxymethyl]-4-(3-methoxy-propyl)-3,4-dihydro-2H-benzo[1,4]oxazine in analogy to method L.

The starting materials are prepared as follows:

### a) 6-[(3R,4S,5S)-4-(4-Methoxyphenyl)-1-(toluene-4-sulfonyl)-5-(3-[1,2,4]triazol-1-yl-propoxy)piperidin-3-yloxymethyl]-4-(3-methoxypropyl)-3,4-dihydro-2H-benzo[1,4]oxazine

0.115 g of 1,2,4-triazole sodium salt [41253-21-8] is added to a solution of 0.184 g of 3-[(3S,4S,5R)-4-(4-methoxyphenyl)-5-[4-(3-methoxypropyl)-3,4-dihydro-2H-benzo[1,4]-oxazin-6-ylmethoxy]-1-(toluene-4-sulfonyl)piperidin-3-yloxy]propyl toluene-4-sulfonate in 2 ml of N,N-dimethylformamide at 0° C. After 15 hours at room temperature, the reaction mixture is diluted with 100 ml of ethyl acetate and washed successively with 15 ml of saturated aqueous sodium bicarbonate solution and 15 ml of brine. The organic phase is dried with sodium sulfate and evaporated. The title compound is obtained as a yellowish resin from the residue by flash chromatography (SiO₂ 60F). Rf=0.10 (EtOAc-heptane 4:1); Rt=4.72 (Gradient I).

### b) 3-[(3S,4S,5R)-4-(4-Methoxyphenyl)-5-[4-(3-methoxypropyl)-3,4-dihydro-2H-benzo[1,4]-oxazin-6-ylmethoxy]-1-(toluene-4-sulfonyl)piperidin-3-yloxy]propyl toluene-4-sulfonate

The title compound is obtained as a colourless oil from 0.195 g of 3-[(3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]propan-1-ol (Example 16a) in analogy to method H. Rf=0.22 (EtOAc-heptane 1:1); Rt=5.68 (Gradient I).

### Example 18

### (R)-1-Methoxy-3-{(3S,4R,5R)-4-[4-(2-methoxyethoxy)phenyl]-5-[4-(3-methoxypropyl)-3,4-dihydro-2H-benzo[1,4]oxazin-6-ylmethoxy]piperidin-3-yloxy}propan-2-ol

The title compound is prepared from 0.200 g of benzyl (3S,4R,5R)-3-((R)-2-hydroxy-3-methoxypropoxy)-4-[4-(2-methoxyethoxy)phenyl]-5-[4-(3-methoxypropyl)-3,4-dihydro-2H-benzo[1,4]oxazin-6-ylmethoxy]piperidine-1-carboxylate in analogy to method B.

The starting materials are prepared as follows:

### a) Benzyl (3S,4R,5R)-3-((R)-2-hydroxy-3-methoxypropoxy)-4-[4-(2-methoxyethoxy)-phenyl]-5-[4-(3-methoxypropyl)-3,4-dihydro-2H-benzo[1,4]oxazin-6-ylmethoxy]piperidine-1-carboxylate

The title compound is obtained as a colourless oil from 0.580 g of benzyl (3S,4S,5R)-3-hydroxy-4-[4-(2-methoxyethoxy)phenyl]-5-[4-(3-methoxypropyl)-3,4-dihydro-2H-benzo[1,4]-oxazin-6-ylmethoxy]piperidin-1-carboxylate and 0.185 g of S-(+)-glycidyl methyl ether [64491-68-5] in analogy to method M. Rf=0.15 (EtOAc-heptane 2:1); Rt=4.70 (Gradient I).

### b) Benzyl (3S,4S,5R)-3-hydroxy-4-[4-(2-methoxyethoxy)phenyl]-5-[4-(3-methoxypropyl)-3,4-dihydro-2H-benzo[1,4]oxazin-6-ylmethoxy]piperidine-1-carboxylate

The title compound is obtained as a colourless oil from 0.584 g of benzyl (3R,4R,5S)-4-(4-hydroxyphenyl)-3-[4-(3-methoxypropyl)-3,4-dihydro-2H-benzo[1,4]oxazin-6-ylmethoxy]-5-triisopropylsilanyloxypiperidine-1-carboxylate (Example 11d) and 0.334 g of 2-methoxyethyl toluene-4-sulfonate [17178-10-8] in analogy to method I. Rf=0.20 (EtOAc-heptane 2:1); Rt=4.62 (Gradient I).

The following compounds are prepared in an analogous manner to the process described in Example 18:

### Examples

### 142 (R)-1-Methoxy-3-{(3S,4R,5R)-4-[4-((S)-4-methoxy-3-methyl-butoxy)-phenyl]-5-[4-(3-methoxy-propyl)-3,4-dihydro-2H-benzo[1,4]oxazin-6-ylmethoxy]-piperidin-3-yloxy}-propan-2-ol

starting from (3S,4S,5R)-3-hydroxy-4-[4-((S)-4-methoxy-3-methyl-butoxy)-phenyl]-5-[4-(3-methoxy-propyl)-3,4-dihydro-2H-benzo[1,4]oxazin-6-ylmethoxy]-piperidine-1-carboxylic acid benzyl ester (Example 140a)

### 143 (S)-1-Methoxy-3-{(3S,4R,5R)-4-[4-((S)-4-methoxy-3-methyl-butoxy)-phenyl]-5-[4-(3-methoxy-propyl)-3,4-dihydro-2H-benzo[1,4]oxazin-6-ylmethoxy]-piperidin-3-yloxy}-propan-2-ol

starting from (3S,4S,5R)-3-hydroxy-4-[4-((S)-4-methoxy-3-methyl-butoxy)-phenyl]-5-[4-(3-methoxy-propyl)-3,4-dihydro-2H-benzo[1,4]oxazin-6-ylmethoxy]-piperidine-1-carboxylic acid benzyl ester (Example 140a) using R-(−)-glycidyl methyl ether [64491-70-9]

### 147 (R)-1-Methoxy-3-{(3S,4R,5R)-4-[4-((R)-4-methoxy-pentyloxy)-phenyl]-5-[4-(3-methoxy-propyl)-3,4-dihydro-2H-benzo[1,4]oxazin-6-ylmethoxy]-piperidin-3-yloxy}-propan-2-ol

starting from (3S,4S,5R)-3-hydroxy-4-[4-((R)-4-methoxy-pentyloxy)-phenyl]-5-[4-(3-methoxy-propyl)-3,4-dihydro-2H-benzo[1,4]oxazin-6-ylmethoxy]-piperidine-1-carboxylic acid benzyl ester (Example 145a)

### 148 (S)-1-Methoxy-3-{(3S,4R,5R)-4-[4-((R)-4-methoxy-pentyloxy)-phenyl]-5-[4-(3-methoxy-propyl)-3,4-dihydro-2H-benzo[1,4]oxazin-6-ylmethoxy]-piperidin-3-yloxy}-propan-2-ol

starting from (3S,4S,5R)-3-hydroxy-4-[4-((R)-4-methoxy-pentyloxy)-phenyl]-5-[4-(3-methoxy-propyl)-3,4-dihydro-2H-benzo[1,4]oxazin-6-ylmethoxy]-piperidine-1-carboxylic acid benzyl ester (Example 145a) using R-(−)-glycidyl methyl ether [64491-70-9]

### Example 19

### (R)-3-{(3S,4R,5R)-4-[4-(2-Methoxyethoxy)phenyl]-5-[4-(3-methoxypropyl)-3,4-dihydro-2H-benzo[1,4]oxazin-6-ylmethoxy]piperidin-3-yloxy}propane-1,2-diol

The title compound is prepared from 0.225 g of benzyl (3S,4R,5R)-3-((R)-2,3-dihydroxy-propoxy)-4-(4-methoxyphenyl)-5-[4-(3-methoxypropyl)-3,4-dihydro-2H-benzo[1,4]oxazin-6-ylmethoxy]piperidine-1-carboxylate in analogy to method B.

The starting materials are prepared as follows:

### a) Benzyl (3S,4R,5R)-3-((R)-2,3-dihydroxypropoxy)-4-(4-methoxyphenyl)-5-[4-(3-methoxy-propyl)-3,4-dihydro-2H-benzo[1,4]oxazin-6-ylmethoxy]piperidine-1-carboxylate

The title compound is obtained as a yellow oil from 4.600 g of benzyl (3S,4R,5R)-3-[(S)-3-(tert-butyldimethylsilanyloxy)-2-hydroxypropoxy)-4-(4-methoxyphenyl)-5-[4-(3-methoxy-propyl)-3,4-dihydro-2H-benzo[1,4]oxazin-6-ylmethoxy]piperidine-1-carboxylate in analogy to method J. Rf=0.11 (EtOAc-heptane 3:1); Rt=4.38 (Gradient I).

### b) Benzyl (3S,4R,5R)-3-[(S)-3-(tert-butyldimethylsilanyloxy)-2-hydroxypropoxy)-4-(4-methoxyphenyl)-5-[4-(3-methoxypropyl)-3,4-dihydro-2H-benzo[1,4]oxazin-6-ylmethoxy]-piperidine-1-carboxylate

The title compound is obtained as a brownish oil from 2.210 g of benzyl (3S,4S,5R)-3-hydroxy-4-(4-methoxyphenyl)-5-[4-(3-methoxypropyl)-3,4-dihydro-2H-benzo[1,4]oxazin-6-yl-methoxy]piperidine-1-carboxylate and 1.86 ml of tert-butyldimethyl((S)-1-oxiranylmethoxy)-silane [123237-62-7] in analogy to method M. Rt=6.14 (Gradient I).

### c) Benzyl (3S,4S,5R)-3-hydroxy-4-(4-methoxyphenyl)-5-[4-(3-methoxypropyl)-3,4-dihydro-2H-benzo[1,4]oxazin-6-ylmethoxy]piperidine-1-carboxylate

The title compound is obtained as a pale yellow oil from 26.59 g of benzyl (3R,4R,5S)-3-hydroxy-4-(4-methoxyphenyl)-5-triisopropylsilanyloxypiperidine-1-carboxylate in analogy to the process described in method D and in Example 1b-c. Rf=0.25 (EtOAc-heptane 3:1); Rt=4.69 (Gradient I).

### d) Benzyl (3R,4R-5S)-3-hydroxy-4-(4-methoxyphenyl)-5-triisopropylsilanyloxypiperidine-1-carboxylate

The title compound is obtained as a yellowish resin from 25 g of benzyl (3R,4R,5S)-3-hydroxy-4-(4-hydroxyphenyl)-5-triisopropylsilanyloxypiperidine-1-carboxylate (Example 11h) in analogy to Example 1e. Rt=6.35 (Gradient I).

### Example 20

### (R)-1-Methoxy-3-{(3S,4R,5R)-4-[4-(3-methoxypropoxy)-phenyl]-5-[4-(3-methoxypropyl)-3,4-dihydro-2H-benzo[1,4]oxazin-6-ylmethoxy]piperidin-3-yloxy}propan-2-ol

The title compound is prepared from 0.260 g of benzyl (3S,4R,5R)-3-((R)-2-hydroxy-3-methoxypropoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate in analogy to method B.

The starting materials are prepared as follows:

### a) Benzyl (3S,4R,5R)-3-((R)-2-hydroxy-3-methoxypropoxy)-4-[4-(3-methoxypropoxy)-phenyl]-5-[4-(3-methoxypropyl)-3,4-dihydro-2H-benzo[1,4]oxazin-6-ylmethoxy]piperidine-1-carboxylate

The title compound is obtained as a pale yellow resin from 0.580 g of benzyl (3S,4R,5R)-3-hydroxy-4-[4-(3-methoxypropoxy)phenyl]-5-[4-(3-methoxypropyl)-3,4-dihydro-2H-benzo-[1,4]oxazin-6-ylmethoxy]piperidine-1-carboxylate and 0.181 g of S-(+)-glycidyl methyl ether [64491-68-5] in analogy to method M. Rf=0.15 (EtOAc-heptane 3:1); Rt=4.92 (Gradient I).

### b) Benzyl (3S,4S,5R)-3-hydroxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate

The title compound is obtained as a yellow oil from 39.10 g of benzyl (3R,4R,5S)-4-[4-(3-methoxypropoxy)phenyl]-3-[4-(3-methoxypropyl)-3,4-dihydro-2H-benzo[1,4]oxazin-6-yl-methoxy]-5-triisopropylsilanyloxypiperidine-1-carboxylate in analogy to method J. Rf=0.17 (EtOAc-heptane 4:1); Rt=4.80 (Gradient I).

### c) Benzyl (3R,4R,5S)-4-[4-(3-methoxypropoxy)phenyl]-3-[4-(3-methoxypropyl)-3,4-dihydro-2H-benzo[1,4]oxazin-6-ylmethoxy]-5-triisopropylsilanyloxypiperidine-1-carboxylate

The title compound is obtained as a yellowish oil from 40.75 g of benzyl (3R,4R,5S)-4-[4-(3-methoxypropoxy)phenyl]-3-[4-(3-methoxypropyl)-3-oxo-3,4-dihydro-2H-benzo[1,4]oxazin-6-ylmethoxy]-5-triisopropylsilanyloxypiperidine-1-carboxylate in analogy to method K. Rf=0.51 (EtOAc-heptane 1:1).

### d) Benzyl (3R,4R,5S)-4-[4-(3-methoxypropoxy)phenyl]-3-[4-(3-methoxypropyl)-3-oxo-3,4-dihydro-2H-benzo[1,4]oxazin-6-ylmethoxy]-5-triisopropylsilanyloxypiperidine-1-carboxylate

The title compound is obtained as a yellowish oil from 33.45 g of benzyl (3R,4R,5S)-3-hydroxy-4-[4-(3-methoxypropoxy)phenyl]-5-triisopropylsilanyloxypiperidine-1-carboxylate in analogy to method D. Rf=0.40 (EtOAc-heptane 1:1); Rt=7.05 (Gradient I).

### e) Benzyl (3R,4R,5S)-3-hydroxy-4-[4-(3-methoxypropoxy)phenyl-5-triisopropylsilanyloxy-piperidine-1-carboxylate

The title compound is obtained as a colourless oil from 32.00 g of benzyl (3R,4R,5S)-3-hydroxy-4-[4-hydroxyphenyl]-5-triisopropylsilanyloxypiperidine-1-carboxylate (Example 11h) in analogy to method F. Rf=0.28 (EtOAc-heptane 1:2); Rt=6.48 (Gradient I).

### Example 21

### 1-Methoxy-3-{(3S,4R,5R)-4-(4-methoxyphenyl-5-[4-(3-methoxypropyl)-3,4-dihydro-2H-benzo-[1,4]oxazin-6-ylmethoxy]piperidin-3-yloxy}propan-2-one

The title compound is prepared from 0.123 g of benzyl (3S,4R,5R)-3-(3-methoxy-2-oxo-propoxy)-4-(4-methoxyphenyl)-5-[4-(3-methoxypropyl)-3,4-dihydro-2H-benzo[1,4]oxazin-6-yl-methoxy]piperidine-1-carboxylate in analogy to method B.

The starting materials are prepared as follows:

### a) Benzyl (3S,4R,5R)-3-(3-methoxy-2-oxopropoxy)-4-(4-methoxyphenyl)-5-[4-(3-methoxy-propyl)-3,4-dihydro-2H-benzo[1,4]oxazin-6-ylmethoxy]piperidine-1-carboxylate

1.00 ml of triethylamine is added dropwise to a solution of 0.820 g of benzyl (3S,4R,5R)-3-((R)-(2-hydroxy-3-methoxypropoxy)-4-(4-methoxyphenyl)-5-[4-(3-methoxypropyl)-3,4-dihydro-2H-benzo[1,4]oxazin-6-ylmethoxy]piperidine-1-carboxylate in 12 ml of dichloro-methane-dimethyl sulfoxide 5:1 at 0-5° C. 0.981 g of sulfur trioxid-pyridine complex is added, and the reaction solution is stirred at room temperature for 16 hours. The reaction solution is poured into ice-water, adjusted to pH 2-3 with 1 M aqueous potassium bisulfate solution and extracted with diethyl ether. The combined organic extracts are washed with brine, dried with sodium sulfate and evaporated. The title compound is obtained as a pale yellow oil from the residue by flash chromatography (SiO₂ 60F). Rf=0.26 (EtOAc-heptane 2:1); Rt=4.97 (Gradient I).

### b) Benzyl (3S,4R,5R)-3-((R)-(2-hydroxy-3-methoxypropoxy)-4-(4-methoxyphenyl)-5-[4-(3-methoxypropyl)-3,4-dihydro-2H-benzo[1,4]oxazin-6-ylmethoxy]piperidine-1-carboxylate

The title compound is obtained as a colourless oil from 1.570 g of benzyl (3S,4S,5R)-3-hydroxy-4-(4-methoxyphenyl)-5-[4-(3-methoxypropyl)-3,4-dihydro-2H-benzo[1,4]oxazin-6-yl-methoxy]piperidine-1-carboxylate (Example 19c) and 0.181 g of S-(+)-glycidyl methyl ether [64491-68-5] in analogy to method M. Rf=0.19 (EtOAc-heptane 2:1); Rt=4.77 (Gradient I).

### Example 22

### 6-[(3R,4R,5S)-5-(2-Methanesulfonylethoxy)-4-(4-methoxyphenyl)piperidin-3-yloxymethyl]-4-(3-methoxypropyl)-3,4-dihydro-2H-benzo[1,4]oxazine

The title compound is prepared from 0.48 g of 6-[(3R,4R,5S)-5-(2-methanesulfonylethoxy)-4-(4-methoxyphenyl)-1-(toluene-4-sulfonyl)piperidin-3-yloxymethyl]-4-(3-methoxypropyl)-3,4-dihydro-2H-benzo[1,4]oxazine in analogy to method L.

The starting material is prepared as follows:

### a) 6-[(3R,4R,5S)-5-(2-Methanesulfonylethoxy)-4-(4-methoxyphenyl)-1-(toluene-4-sulfonyl)-piperidin-3-yloxymethyl]-4-(3-methoxypropyl)-3,4-dihydro-2H-benzo[1,4]oxazine

0.472 g of (3S,4S,5R)-4-(4-methoxyphenyl)-5-[4-(3-methoxypropyl)-3,4-dihydro-2H-benzo-[1,4]oxazin-6-ylmethoxy]-1-(toluene-4-sulfonyl)piperidin-3-ol (Example 1b) and 0.834 g of methyl vinyl sulfone [3680-02-2] are reacted in analogy to Example 14b. The title compound is obtained as a yellowish oil. Rf=0.35 (EtOAc-heptane 2:1); Rt=4.91 (Gradient I).

### Example 23

### 4-{(3S,4R,5R)-4-(4-Methoxyphenyl)-5-[4-(3-methoxypropyl)-3,4-dihydro-2H-benzo[1,4]-oxazin-6-ylmethoxy]piperidin-3-yloxymethyl}tetrahydropyran-4-ol

The title compound is prepared from 0.270 g of 4-[(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)-piperidin-3-yloxymethyl]tetrahydropyran-4-ol in analogy to method L.

The starting material is prepared as follows:

### a) 4-[(3S,4R,5R)-4-(4-Methoxyphenyl)-5-[4-(3-methoxypropyl)-3,4-dihydro-2H-benzo[1,4]-oxazin-6-ylmethoxy]-1-(toluene-4-sulfonyl)piperidin-3-yloxymethyl]tetrahydropyran-4-ol

0.04 g of sodium hydride (60% dispersion in oil) is added to a solution of 0.40 g of (3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-ol (Example 1b) in 6 ml of 1,3-dimethyl-3,4,5,6-tetrahydro-2(1H)-pyrimidone (DMPU). The mixture is stirred at 60° C. for 10 minutes and then a solution of 0.174 g of 1,6-dioxaspiro[2,5]octane [185-72-8] in 2 ml of DMPU is added. After one hour at 60° C., the reaction mixture is cooled at room temperature and diluted with 200 ml of tert-butyl methyl ether. The solution is washed successively with 20 ml of 1 N HCl, with 30 ml unsaturated aqueous sodium bicarbonate solution and 20 ml of brine. The organic phase is dried with sodium sulfate and evaporated. The title compound is obtained as a yellowish resin from the residue by flash chromatography (SiO₂ 60F). Rf=0.23 (EtOAc-heptane 2:1); Rt=4.87 (Gradient I).

The following compound is prepared in an analogous manner to the process described in Example 23:

### Example 137

### 6-[(3R,4R,5S)-5-[1-(2-Methoxy-ethoxy)-cyclopentylmethoxy]-4-(4-methoxy-phenyl)-piperidin-3-yloxymethyl]-4-(3-methoxy-propyl)-3,4-dihydro-2H-benzo[1,4]oxazine

using 1-iodomethyl-1-(2-methoxy-ethoxy)-cyclopentane

The starting material is prepared as follows:

### a) 1-Iodomethyl-1-(2-methoxy-ethoxy)-cyclopentane

To a solution of 25.98 mmol of 2-methoxyethanol [109-86-4] and 23.62 mmol of methylene-cyclopentane [1528-30-9] in dry actetinitile is added 25.98 mmol of N-iodosuccinimid in one portion. The reaction mixture is stirred for 20 hours at room temperature under exclusion of light. The reaction mixture is poured into brine, extracted with diethyl ether (2×) and evaporated to a concentrated solution. The title compound is obtained as a yellow oil from the residue by flash chromatography (SiO₂ 60F). Rf=0.42 (EtOAc-heptane 1:4).

### Example 24

### 2-{(3S,4R,5R)-4-(4-Methoxyphenyl)-5-[4-(3-methoxypropyl)-3,4-dihydro-2H-benzo[1,4]-oxazin-6-ylmethoxy]piperidin-3-yloxy}-N,N-dimethylacetamide

The title compound is prepared from 0.140 g of 2-[(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)-piperidin-3-yloxy]-N,N-dimethylacetamide in analogy to method L.

The starting material is prepared as follows:

### a) 2-[(3S,4R,5R)-4-(4-Methoxyphenyl)-5-[4-(3-methoxypropyl)-3,4-dihydro-2H-benzo[1,4]-oxazin-6-ylmethoxy]-1-(toluene-4-sulfonyl)piperidin-3-yloxy]-N,N-dimethylacetamide

A solution of 0.145 g of methyl [(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]acetate (Example 8c) in 5 ml of dimethylamide (33% in ethanol) is stirred at 50° C. for 24 hours and then evaporated to dryness. The title compound is obtained as a yellow oil from the residue. Rt=4.82 (Gradient I).

### Example 25

### (R,S)-1-Methoxy-3-{(3S,4R,5R)-4-(4-methoxyphenyl-5-[4-(3-methoxypropyl)-3,4-dihydro-2H-benzo[1,4]oxazin-6-ylmethoxy]piperidin-3-yloxy]-2-methylpropan-2-ol

The title compound is prepared from 0.381 g of benzyl (3S,4R,5R)-3-((R,S)-2-hydroxy-3-methoxy-2-methylpropoxy)-4-(4-methoxyphenyl)-5-[4-(3-methoxypropyl)-3,4-dihydro-2H-benzo[1,4]oxazin-6-ylmethoxy]piperidine-1-carboxylate in analogy to method B.

The starting material is prepared as follows:

### a) Benzyl (3S,4R,5R)-3-((R,S)-2-hydroxy-3-methoxy-2-methylpropoxy)-4-(4-methoxy-phenyl)-5-[4-(3-methoxypropyl)-3,4-dihydro-2H-benzo[1,4]oxazin-6-ylmethoxy]piperidine-1-carboxylate

0.30 ml of methylmagnesium bromide solution (35% in diethyl ether) is added to a solution of 0.542 g of benzyl (3S,4R,5R)-3-(3-methoxy-2-oxopropoxy)-4-(4-methoxyphenyl)-5-[4-(3-methoxypropyl)-3,4-dihydro-2H-benzo[1,4]oxazin-6-ylmethoxy]piperidine-1-carboxylate (Example 21a) in 5 ml of dry tetrahydrofuran at room temperature, and the mixture is stirred at room temperature for 2 hours. The reaction mixture is poured into 1 M aqueous potassium bisulfate solution and extracted with tert-butyl methyl ether. The combined organic extracts are washed with brine, dried with sodium sulfate and evaporated. The title compound is obtained as a pale yellow oil from the residue by flash chromatography (SiO₂ 60F). Rf=0.15 (EtOAc-heptane 2:1); Rt=5.03 (Gradient I).

### Example 26

### (R)-2-Fluoro-3-{(3S,4R,5R)-4-(4-methoxyphenyl)-5-[4-(3-methoxypropyl)-3,4-dihydro-2H-benzo[1,4]oxazin-6-ylmethoxy]piperidin-3-yloxy]propan-1-ol

0.10 ml of 2M aqueous HCl is added to a solution of 0.135 g of benzyl (3S,4R,5R)-3((R)-3-benzyloxy-2-fluoropropoxy)-4-(4-methoxyphenyl)-5-[4-(3-methoxypropyl)-3,4-dihydro-2H-benzo[1,4]oxazin-6-ylmethoxy]piperidine-1-carboxylate in 6 ml of 1:1 tetrahydrofuran-methanol, and hydrogenation is carried out in the presence of 60 mg of 10% Pd/C at 20° C. for 6 hours. The reaction mixture is clarified by filtration through Hyflo and the filtrate is evaporated. The title compound is obtained from the residue by flash chromatography (SiO₂ 60F).

The starting materials are prepared as follows:

### a) Benzyl (3S,4R,5R)-3((R)-3-benzyloxy-2-fluoropropoxy)-4-(4-methoxyphenyl)-5-[4-(3-methoxypropyl)-3,4-dihydro-2H-benzo[1,4]oxazin-6-ylmethoxy]piperidine-1-carboxylate

A solution of 0.081 ml of diethylaminosulfur trifluoride in 5 ml of dry dichloromethane is cooled to −78° C. A solution of 0.418 g of benzyl (3S,4R,5R)-3((S)-3-benzyloxy-2-hydroxy-propoxy)-4-(4-methoxyphenyl)-5-[4-(3-methoxypropyl)-3,4-dihydro-2H-benzo[1,4]oxazin-6-ylmethoxy]piperidine-1-carboxylate in 5 ml of dichloromethane is added dropwise at this temperature. The reaction solution is stirred at −78° C. for 1 hour and then warmed to room temperature over 3-4 hours. The reaction mixture is poured into a mixture of ice and saturated aqueous sodium bicarbonate solution and then extracted with dichloromethane. The combined organic phases are washed with water, dried with sodium sulfate and evaporated. The title compound is obtained as a pale yellow oil from the residue by flash chromatography (SiO₂ 60F). Rf=0.29 (EtOAc-heptane 1:1); Rt=5.70 (Gradient I).

### b) Benzyl (3S,4R,5R)-3((S)-3-benzyloxy-2-hydroxypropoxy)-4-(4-methoxyphenyl)-5-[4-(3-methoxypropyl)-3,4-dihydro-2H-benzo[1,4]oxazin-6-ylmethoxy]piperidine-1-carboxylate

The title compound is obtained as a yellow oil from 0.406 g of benzyl (3S,4S,5R)-3-hydroxy-4-(4-methoxyphenyl)-5-[4-(3-methoxypropyl)-3,4-dihydro-2H-benzo[1,4]oxazin-6-ylmethoxy]-piperidine-1-carboxylate (Example 19c) and 0.257 g of (R)-2-benzyloxymethyloxirane [14618-80-5] in analogy to method M. Rf=0.38 (EtOAc-heptane 3:1); Rt=5.33 (Gradient I).

The following compound is prepared in an analogous manner to the process described in Example 26:

### Example 27

### (S)-2-Fluoro-3-{(3S,4R,5R)-4-(4-methoxyphenyl)-5-[4-(3-methoxypropyl)-3,4-dihydro-2H-benzo[1,4]oxazin-6-ylmethoxy]piperidin-3-yloxy]propan-1-ol

### Example 28

### 2-{(3S,4R,5R)-4-(4-Methoxyphenyl)-5-[4-(3-methoxypropyl)-3,4-dihydro-2H-benzo[1,4]-oxazin-6-ylmethoxy]piperidin-3-yloxy}-1-pyrrolidin-1-yl-ethanone

The title compound is prepared from 0.29 g of 2-[(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)-piperidin-3-yloxy]-1-pyrrolidin-1-yl-ethanone in analogy to method L.

The starting materials are prepared as follows:

### a) 2-[(3S,4R,5R)-4-(4-Methoxyphenyl)-5-[4-(3-methoxypropyl)-3,4-dihydro-2H-benzo[1,4]-oxazin-6-ylmethoxy]-1-(toluene-4-sulfonyl)piperidin-3-yloxy]-1-pyrrolidin-1-yl-ethanone

A solution of 0.37 g of methyl [(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]acetate and 0.39 g of pyrrolidine is heated at 75° C. for 2 hours. The solvent is evaporated in vacuo and the title compound is obtained in the form of white crystals from the residue by flash chromatography (SiO₂ 60F). Rf=0.22 (EtOAc-heptane 3:1); Rt=4.94 (Gradient I).

### b) Methyl [(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]acetate

0.53 g of sodium hydride (60% dispersion in oil) is added to a solution of 3.2 g of ((3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-ol (Example 1b) in 40 ml of tetrahydrofuran, and the mixture is stirred at room temperature for 1 hour. It is then cooled to −5° C. and 2.49 g of methyl bromoacetate are added dropwise over the course of one hour. The reaction mixture is stirred at −5° C. for 3 hours and then warmed to room temperature. It is then diluted with tert-butyl methyl ether and poured into 0.5M aqueous HCl. The resulting mixture is extracted three times with tert-butyl methyl ether. The combined organic phases are washed with brine, dried with sodium sulfate and evaporated. The title compound is obtained as a yellow oil from the residue by flash chromatography (SiO₂ 60F). Rf=0.5 (EtOAc-heptane 2:1); Rt=5.14 (Gradient I).

The following compounds are prepared in an analogous manner to the process described in Example 28:

### Examples

### 32 2-{(3S,4R,5R)-4-(4-Methoxyphenyl)-5-[4-(3-methoxypropyl)-3,4-dihydro-2H-benzo[1,4]oxazin-6-ylmethoxy]piperidin-3-yloxy}-N-(tetrahydro-pyran-4-yl)acetamide

### 34 2-{(3S,4R,5R)-4-(4-Methoxyphenyl)-5-[4-(3-methoxypropyl)-3,4-dihydro-2H-benzo[1,4]oxazin-6-ylmethoxy]piperidin-3-yloxy}-1-morpholin-4-ylethanone

### Example 29

### (S)-1-Methoxy-3-{(3S,4R,5R)-4-[4-(3-methoxypropoxy)phenyl]-5-[4-(3-methoxypropyl)-3,4-dihydro-2H-benzo[1,4]oxazin-6-ylmethoxy]piperidin-3-yloxy}propan-2-ol

The title compound is prepared from 0.495 g of benzyl (3S,4R,5R)-3-((S)-2-hydroxy-3-methoxypropoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate in analogy to method B.

The starting material is prepared as follows:

### a) Benzyl (3S,4R,5R)-3-((S)-2-hydroxy-3-methoxypropoxy)-4-[4-(3-methoxypropoxy)-phenyl]-5-[4-(3-methoxypropyl)-3,4-dihydro-2H-benzo[1,4]oxazin-6-ylmethoxy]piperidine-1-carboxylate

0.635 g of benzyl (3S,4S,5R)-3-hydroxy-4-[4-(3-methoxypropoxy)phenyl]-5-[4-(3-methoxy-propyl)-3,4-dihydro-2H-benzo[1,4]oxazin-6-ylmethoxy]piperidine-1-carboxylate (Example 20b) and 0.202 g of R-(−)-glycidyl methyl ether [64491-70-9] are reacted in analogy to method M. The title compound is obtained as a yellow oil. Rf=0.06 (EtOAc-heptane 1:1); Rt=4.91 (Gradient I).

### Example 30

### 6-[(3R,4S,5S)-4-(4-Methoxyphenyl)-5-(3-morpholin-4-41-propoxy)piperidin-3-yloxymethyl]-4-(3-methoxypropyl)-3,4-dihydro-2H-benzo[1,4]oxazine

The title compound is prepared from 0.26 g of 6-[(3R,4S,5S)-4-(4-methoxyphenyl)-5-(3-morpholin-4-yl-propoxy)-1-(toluene-4-sulfonyl)piperidin-3-yloxymethyl]-4-(3-methoxypropyl)-3,4-dihydro-2H-benzo[1,4]oxazine in analogy to method L.

The starting materials are prepared as follows:

### a) 6-[(3R,4S,5S)-4-(4-Methoxyphenyl)-5-(3-morpholin-4-ylpropoxy)-1-(toluene-4-sulfonyl)-piperidin-3-yloxymethyl]-4-(3-methoxypropyl)-3,4-dihydro-2H-benzo[1,4]oxazine

0.034 ml of acetic acid, 0.054 ml of morpholine and 0.181 g of sodium triacetoxyborohydride are successively added to a solution of 0.35 g of 3-[(3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)-piperidin-3-yloxy]propionaldehyde in 3.5 ml of tetrahydrofuran. After one hour at room temperature, the reaction mixture is poured into 30 ml of ice-water and extracted with tert-butyl methyl ether (2×30 ml). The combined organic phases are washed successively with 30 ml of water and 30 ml of brine, dried with sodium sulfate and evaporated. The title compound is obtained as a colourless resin from the residue by flash chromatography (SiO₂ 60F). Rf=0.33 (dichloromethane-methanol-25% conc. ammonia=200:10:1); Rt=4.46 (Gradient I).

### b) 3-[(3S,4S,5R)-4-(4-Methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]propionaldehyde

0.328 g of pyridine-sulfur trioxide complex is added to a solution of 0.405 g of 3-[(3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]-propan-1-ol (Example 16a) and 0.433 ml of triethylamine in 6 ml of 1:5 dimethyl sulfoxide-dichloromethane at 0° C. After 3 hours at 0° C., the reaction mixture is stirred at room temperature. After 1 hour, a further 0.1 g of pyridine-sulfur trioxide complex is added. After a further 1 hour, the reaction mixture is poured into 10 ml of ice-water, adjusted to pH 2.5 with 0.5 ml of 1 N potassium bisulfate solution and extracted with diethyl ether (3×20 ml). The combined organic phases are washed successively with 20 ml of water and 20 ml of 5% aqueous sodium bicarbonate solution, dried with sodium sulfate and evaporated. The crude title compound is obtained as a yellowish resin from the residue. Rf=0.09 (EtOAc-heptane 1:2); Rt=5.04 (Gradient I).

### Example 31

### 6-[(3R,4R,5S)-4-(4-Methoxyphenyl)-5-(2-morpholin-4-ylethoxy)piperidin-3-yloxymethyl]-4-(3-methoxypropyl)-3,4-dihydro-2H-benzo[1,4]oxazine

The title compound is prepared from 0.11 g of 6-[(3R,4R,5S)-4-(4-methoxyphenyl)-5-(2-morpholin-4-ylethoxy)-1-(toluene-4-sulfonyl)piperidin-3-yloxymethyl]-4-(3-methoxypropyl)-3,4-dihydro-2H-benzo[1,4]oxazine in analogy to method L.

The starting materials are prepared as follows:

### a) 6-[(3R,4R,5S)-4-(4-Methoxyphenyl)-5-(2-morpholin-4-ylethoxy)-1-(toluene-4-sulfonyl)-piperidin-3-yloxymethyl]-4-(3-methoxypropyl)-3,4-dihydro-2H-benzo[1,4]oxazine

0.122 g of [(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]acetaldehyde and 0.0154 g of morpholine are reacted in analogy to Example 30a. The title compound is obtained as a yellowish resin. Rf=0.38 (dichloromethane-methanol-25% conc. ammonia=200:10:1); Rt=4.45 (Gradient I).

### b) [(3S,4R,5R)-4-(4-Methoxyphenyl)-5-[4-(3-methoxypropyl)-3,4-dihydro-2H-benzo[1,4]-oxazin-6-ylmethoxy]-1-(toluene-4-sulfonyl)piperidin-3-yloxy]acetaldehyde

0.135 g of 2-[(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]ethanol is reacted in analogy to Example 30b. The title compound is obtained as a yellowish resin. Rf=0.05 (EtOAc-heptane 1:2); Rt=4.67 (Gradient I).

### c) 2-[(3S,4R,5R)-4-(4-Methoxyphenyl)-5-[4-(3-methoxypropyl)-3,4-dihydro-2H-benzo[1,4]-oxazin-6-ylmethoxy]-1-(toluene-4-sulfonyl)piperidin-3-yloxy]ethanol

0.19 g of 6-[(3R,4R,5S)-4-(4-methoxyphenyl)-1-(toluene-4-sulfonyl)-5-(2-triisopropylsilanyl-oxyethoxy)piperidin-3-yloxymethyl]-4-(3-methoxypropyl)-3,4-dihydro-2H-benzo[1,4]oxazine is reacted in analogy to Example 1b. The title compound is obtained as a yellowish resin. Rf=0.31 (EtOAc-heptane 1:2); Rt=4.77 (Gradient I).

### d) 6-[(3R,4R,5S)-4-(4-Methoxyphenyl)-1-(toluene-4-sulfonyl)-5-(2-triisopropylsilanyloxy-ethoxy)piperidin-3-yloxymethyl]-4-(3-methoxypropyl)-3,4-dihydro-2H-benzo[1,4]oxazine

0.044 g of sodium hydride (60% dispersion in oil) is added to a solution of 0.50 g of (3S,4S,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-ol (Example 1b) and 0.303 g of (2-iodoethoxy)-triisopropylsilane [93550-77-7] in 5 ml of tetrahydrofuran while stirring at room temperature. After 4 hours at 50° C., 0.303 g of (2-iodoethoxy)triisopropylsilane and 0.044 g of sodium hydride (60% dispersion in oil) are again added to the mixture. After 15 hours at 50° C., the reaction mixture is diluted at room temperature with tert-butyl methyl ether. The solution is washed successively with aqueous sodium bicarbonate solution, with water and brine, dried with sodium sulfate and evaporated. The title compound is obtained as a yellowish resin from the residue by flash chromatography (SiO₂ 60F). Rf=0.25 (EtOAc-heptane 1:2).

### Example 33

### 6-{(3R,4S,5S)-5-(3-Methoxypropoxy)-4-[4-(3-methoxypropoxy)phenyl]piperidin-3-yloxy-methyl}-4-(3-methoxypropyl)-3,4-dihydro-2H-benzo[1,4]oxazine

The title compound is obtained from 0.218 g of benzyl (3S,4S,5R)-3-(3-methoxypropoxy)-4-[4-(3-methoxypropoxy)phenyl]-5-[4-(3-methoxypropyl)-3,4-dihydro-2H-benzo[1,4]oxazin-6-yl-methoxy]piperidine-1-carboxylate in analogy to method B.

The starting material is prepared as follows:

### a) Benzyl (3S,4S,5R)-3-(3-methoxypropoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate

0.090 g of sodium hydride (60% dispersion in oil) is added to a solution of 0.955 g of benzyl (3S,4S,5R)-3-hydroxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate (Example 20b) in 7.0 ml of N,N-dimethylformamide while stirring at 0° C. The reaction mixture is stirred at 0° C. for 1 hour. 0.258 g of 1-bromo-3-methoxypropane and 0.023 g of sodium iodide are successively added to the mixture. The reaction mixture is stirred at room temperature for 16 hours and then poured into 50 ml of water and extracted with tert-butyl methyl ether (3×50 ml). The organic phases are washed successively with water (2×50 ml) and brine (50 ml), dried with sodium sulfate and evaporated. The title compound is obtained as a colourless oil from the residue by flash chromatography (SiO₂ 60F). Rf=0.23 (EtOAc-heptane 3:1); Rt=5.45 (Gradient I).

### Example 35

### 3-{(3S,4S,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}propan-1-ol

The title compound is prepared from 0.300 g of benzyl (3S,4S,5R)-3-(3-hydroxypropoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate in analogy to method B.

The starting materials are prepared as follows:

### a) Benzyl (3S,4S,5R)-3-(3-hydroxypropoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate

2.95 g of benzyl (3R,4S,5S)-4-[4-(3-methoxypropoxy)phenyl]-3-[4-(3-methoxypropyl)-3,4-dihydro-2H-benzo[1,4]oxazin-6-ylmethoxy]-5-(3-triisopropylsilanyloxypropoxy)piperidine-1-carboxylate are reacted in analogy to method J. The title compound is obtained as a colour-less oil. Rf=0.11 (EtOAc-heptane 2:1); Rt=4.83 (Gradient I).

### b) Benzyl (3R,4S,5S)-4-[4-(3-methoxypropoxy)phenyl]-3-[4-(3-methoxypropyl)-3,4-dihydro-2H-benzo[1,4]oxazin-6-ylmethoxy]-5-(3-triisopropylsilanyloxypropoxy)piperidine-1-carboxylate

4.0 g of benzyl (3S,4S,5R)-3-hydroxy-4-[4-(3-methoxypropoxy)phenyl]-5-[4-(3-methoxy-propyl)-3,4-dihydro-2H-benzo[1,4]oxazin-6-ylmethoxy]piperidine-1-carboxylate (Example 20b) and 2.07 g of (3-bromopropoxy)triisopropylsilane are reacted in analogy to Example 31d. The title compound is obtained as a colourless oil. Rf=0.56 (EtOAc-heptane 2:1).

### Example 36

### (R)-1-{(3S,4R,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}propan-2-ol

The title compound is obtained from 0.340 g of benzyl (3R,4R,5S)-4-[4-(3-methoxypropoxy)-phenyl]-3-[4-(3-methoxypropyl)-3,4-dihydro-2H-benzo[1,4]oxazin-6-ylmethoxy]-5-((R)-1-oxiranylmethoxy)piperidine-1-carboxylate in analogy to method B.

The starting material is prepared as follows:

### a) Benzyl (3R,4R,5S)-4-[4-(3-methoxypropoxy)phenyl]-3-[4-(3-methoxypropyl)-3,4-dihydro-2H-benzo[1,4]oxazin-6-ylmethoxy]-5-((R)-1-oxiranylmethoxy)piperidine-1-carboxylate

0.065 g of sodium hydride (60% dispersion in oil) is added to a solution of 0.860 g of benzyl (3S,4S,5R)-hydroxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate (Example 20b) in 5 ml of tetrahydrofuran. The mixture is stirred at 40° C. for 45 minutes. A solution of 0.625 g of (R)-1-oxiranylmethyl toluene-4-sulfonate [113826-06-5] in 3 ml of tetrahydrofuran is added, and the reaction mixture is heated at 50° C. for 3 hours. The reaction mixture is poured into saturated aqueous sodium bicarbonate solution, and the mixture is extracted with tert-butyl methyl ether. The combined organic extracts are washed with brine, dried with sodium sulfate and evaporated. The title compound is obtained as a colourless oil from the residue by flash chromatography (SiO₂ 60F). Rf=0.25 (EtOAc-heptane 2:1); Rt=5.26 (Gradient I).

The following compounds are prepared in an analogous manner to the process described in Example 36:

### Examples

### 44 (S)-1-{(3S,4R,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}propan-2-ol

starting from benzyl (3S,4S,5R)-hydroxy-4-[4-(3-methoxypropoxy)phenyl]-5-[4-(3-methoxy-propyl)-3,4-dihydro-2H-benzo[1,4]oxazin-6-ylmethoxy]piperidine-1-carboxylate (Example 20b) and 0.622 g of (S)-1-oxiranylmethyl toluene-4-sulfonate [70987-78-9]

### 140 (R)-1-{(3S,4R,5R)-4-[4-((S)-4-Methoxy-3-methyl-butoxy)-phenyl]-5-[4-(3-methoxy-propyl)-3,4-dihydro-2H-benzo[1,4]oxazin-6-ylmethoxy]-piperidin-3-yloxy}-propan-2-ol

starting from (3S,4S,5R)-3-hydroxy-4-[4-((S)-4-methoxy-3-methyl-butoxy)-phenyl]-5-[4-(3-methoxy-propyl)-3,4-dihydro-2H-benzo[1,4]oxazin-6-ylmethoxy]-piperidine-1-carboxylic acid benzyl ester.

The starting material is prepared as follows:

### a) (3S,4S,5R)-3-Hydroxy-4-[4-((S)-4-methoxy-3-methyl-butoxy)-phenyl]-5-[4-(3-methoxy-propyl)-3,4-dihydro-2H-benzo[1,4]oxazin-6-ylmethoxy]-piperidine-1-carboxylic acid benzyl ester

The title compound is obtained from benzyl (3R,4R,5S)-3-hydroxy-4-(4-hydroxyphenyl)-5-triisopropylsilanyloxypiperidine-1-carboxylate (Example 11h) in analogy to the process described in Example 20b, c, d, e using toluene-4-sulfonic acid (S)-4-methoxy-3-methyl-butyl ester (Example 144a). The title compound is identified from the residue on the basis of the Rf by flash chromatography (SiO₂ 60F).

### 141 (S)-1-{(3S,4R,5R)-4-[4-((S)-4-Methoxy-3-methyl-butoxy)-phenyl]-5-[4-(3-methoxy-propyl)-3,4-dihydro-2H-benzo[1,4]oxazin-6-ylmethoxy]-piperidin-3-yloxy}-propan-2-ol

starting from (3S,4S,5R)-3-hydroxy-4-[4-((S)-4-methoxy-3-methyl-butoxy)-phenyl]-5-[4-(3-methoxy-propyl)-3,4-dihydro-2H-benzo[1,4]oxazin-6-ylmethoxy]-piperidine-1-carboxylic acid benzyl ester (Example 140a) using (S)-1-oxiranymethylester [70987-78-9].

### 145 (R)-1-{(3S,4R,5R)-4-[4-((R)-4-Methoxy-pentyloxy)-phenyl]-5-[4-(3-methoxy-propyl)-3,4-dihydro-2H-benzo[l 4]oxazin-6-ylmethoxy]-piperidin-3-yloxy}-propan-2-ol

starting from (3S,4S,5R)-3-hydroxy-4-[4-((R)-4-methoxy-pentyloxy)-phenyl]-5-[4-(3-methoxy-propyl)-3,4-dihydro-2H-benzo[1,4]oxazin-6-ylmethoxy]-piperidine-1-carboxylic acid benzyl ester.

The starting material is prepared as follows:

### a) (3S,4S,5R)-3-Hydroxy-4-[4-((R)-4-methoxy-pentyloxy)-phenyl]-5-[4-(3-methoxy-propyl)-3,4-dihydro-2H-benzo[1,4]oxazin-6-ylmethoxy]-piperidine-1-carboxylic acid benzyl ester

The title compound is obtained from benzyl (3R,4R,5S)-3-hydroxy-4-(4-hydroxyphenyl)-5-triisopropylsilanyloxypiperidine-1-carboxylate (Example 11h) in analogy to the procedure described in Example 20b, c, d, e using toluene-4-sulfonic acid (R)-4-methoxy-pentyl ester (Example 149a). The title compound is identified from the residue on the basis of the Rf by flash chromatography (SiO₂ 60F).

### 146 (S)-1-{(3S,4R,5R)-4-[4-((R)-4-Methoxy-3-methyl-butoxy)-phenyl]-5-[4-(3-methoxy-propyl)-3,4-dihydro-2H-benzo[1,4]oxazin-6-ylmethoxy]-piperidin-3-yloxy}-propan-2-ol

starting from (3S,4S,5R)-3-hydroxy-4-[4-((R)-4-methoxy-pentyloxy)-phenyl]-5-[4-(3-methoxy-propyl)-3,4-dihydro-2H-benzo[1,4]oxazin-6-ylmethoxy]-piperidine-1-carboxylic acid benzyl ester (Example 145a) using (S)-1-oxiranymethylester [70987-78-9].

### Example 37

### (3-{(3S,4S,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}propyl)dimethylamine

The title compound is prepared from 0.190 g of benzyl (3S,4S,5R)-3-(3-dimethylamino-propoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate in analogy to method B.

The starting materials are prepared as follows:

### a) Benzyl (3S,4S,5R)-3-(3-dimethylaminopropoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate

0.048 ml of acetic acid, 0.08 g of dimethylamine (in 0.5 ml of tetrahydrofuran) and 0.181 g of sodium triacetoxyborohydride are successively added to a solution of 0.530 g of benzyl (3R,4S,5S)-4-[4-(3-methoxypropoxy)phenyl]-3-[4-(3-methoxypropyl)-3,4-dihydro-2H-benzo[1,4]oxazin-6-ylmethoxy]-5-(3-oxopropoxy)piperidine-1-carboxylate in 5.3 ml of ethyl acetate at room temperature. The reaction mixture is stirred at room temperature for 2 hours and then poured into 1 M sodium bicarbonate solution (30 ml), and extracted with tert-butyl methyl ether (2×30 ml). The organic phases are washed successively with water (30 ml) and brine (30 ml), dried with sodium sulfate and evaporated. The title compound is obtained as a colourless oil from the residue by flash chromatography (SiO₂ 60F). Rf=0.22 (dichloro-methane-methanol-25% conc. ammonia=200:20:1); Rt=4.50 (Gradient I).

### b) Benzyl (3R,4S,5S)-4-[4-(3-methoxypropoxy)phenyl]-3-[4-(3-methoxypropyl)-3,4-dihydro-2H-benzo[1,4]oxazin-6-ylmethoxy]-5-(3-oxopropoxy)piperidine-1-carboxylate

0.46 g of pyridine-sulfur trioxide is added in portions to a solution of 0.600 g of benzyl (3S,4S,5R)-3-(3-hydroxypropoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate (Example 35a) in 9 ml of 1:5 dimethyl sulfoxide-dichloromethane at 0° C. The reaction mixture is stirred at 0° C. for 30 minutes and then poured into ice-water (10 ml). 1M potassium bisulfite solution (0.5 ml) is added to the mixture, which is then extracted with diethyl ether (3×20 ml). The organic phases are washed successively with water (30 ml) and 0.5M sodium bicarbonate solution (20 ml), dried with sodium sulfate and evaporated. The crude title compound is obtained as a yellowish oil. Rt=5.14 (Gradient I).

### Example 38

### 6-[(3R,4S,5S)-4-[4-(3-Methoxypropoxy)phenyl]-5-(3-[1,2,4]-triazol-1-ylpropoxy)piperidin-3-yloxymethyl]-4-(3-methoxypropyl)-3,4-dihydro-2H-benzo[1,4]oxazine

The title compound is prepared from 0.390 g of benzyl (3R,4S,5S)-4-[4-(3-methoxypropoxy)-phenyl]-3-[4-(3-methoxypropyl)-3,4-dihydro-2H-benzo[1,4]oxazin-6-ylmethoxy]-5-(3-[1,2,4]triazol-1-ylpropoxy)piperidine-1-carboxylate in analogy to method B.

The starting materials are prepared as follows:

### a) Benzyl 3R,4S,5S)-4-[4-(3-methoxypropoxy)phenyl]-3-[4-(3-methoxypropyl)-3,4-dihydro-2H-benzo[1,4]oxazin-6-ylmethoxy]-5-(3-[1,2,4]-triazol-1-yl-propoxy)piperidine-1-carboxylate

0.305 g of 1,2,4-triazole sodium salt [41253-21-8] is added to a solution of 0.510 g of benzyl (3R,4S,5S)-4-[4-(3-methoxypropoxy)phenyl]-3-[4-(3-methoxypropyl)-3,4-dihydro-2H-benzo[1,4]oxazin-6-ylmethoxy]-5-[3-(toluene-4-sulfonyloxy)propoxy]piperidine-1-carboxylate in 6 ml of N,N-dimethylformamide at 0° C., and the mixture is stirred at room temperature for 3 hours. The reaction mixture is poured into ice-water and extracted with tert-butyl methyl ether. The organic phases are washed with water and brine, dried with sodium sulfate and evaporated. The title compound is obtained as a colourless oil from the residue by flash chromatography (SiO₂ 60F). Rf=0.63 (dichloromethane-methanol-25% conc. ammonia=200:20:1). Rt=4.80 (Gradient I).

### b) Benzyl (3R,4S,5S)-4-[4-(3-methoxypropoxy)phenyl]-3-[4-(3-methoxypropyl)-3,4-dihydro-2H-benzo[1,4]oxazin-6-ylmethoxy]-5-[3-(toluene-4-sulfonyloxy)propoxy]piperidine-1-carboxylate

0.500 g of benzyl (3S,4S,5R)-3-(3-hydroxypropoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate (Example 35a) is reacted in analogy to method H. The title compound is obtained as a yellowish oil. Rf=0.16 (EtOAc-heptane 1:1). Rt=5.78 (Gradient I).

### Example 39

### N,N-Diethyl-2-{(3S,4R,5R)-4-[4-(3-methoxypropoxy)phenyl]-5-[4-(3-methoxypropyl)-3,4-dihydro-2H-benzo[1,4]oxazin-6-ylmethoxy]piperidin-3-yloxy}acetamide

The title compound is prepared from 0.44 g of benzyl (3S,4R,5R)-3-diethylcarbamoylmethoxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate in analogy to method B.

The starting materials are prepared as follows:

### a) Benzyl (3S,4R,5R)-3-diethylcarbamoylmethoxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate

0.53 ml of propanephosphonic anhydride [68957-94-8, T3P] (50% in ethyl acetate) is added to a solution of 0.517 g of benzyl (3S,4R,5R)-3-carboxymethoxy-4-[4-(3-methoxypropoxy)-phenyl]-5-[4-(3-methoxypropyl)-3,4-dihydro-2H-benzo[1,4]oxazin-6-ylmethoxy]piperidine-1-carboxylate, 0.066 g of diethylamine and 0.52 ml of triethylamine in 8 ml of dichloromethane at 0° C., and the mixture is stirred at room temperature for 16 hours. The reaction mixture is diluted with dichloromethane, and 0.1M aqueous HCl is added. The phases are separated and the aqueous phase is extracted twice more with dichloromethane. The combined organic phases are washed with brine, dried with sodium sulfate and evaporated. The title compound is obtained as yellow oil from the residue by flash chromatography (SiO₂ 60F). Rf=0.2 (EtOAc-heptane 5:1); Rt=5.20 (Gradient I).

### b) Benzyl (3S,4R,5R)-3-carboxymethoxy-4-[4-(3-methoxypropoxy)phenyl]-5-[4-(3-methoxy-propyl)-3,4-dihydro-2H-benzo[1,4]oxazin-6-ylmethoxy]piperidine-1-carboxylate

8 ml of a 1.5M aqueous lithium hydroxide solution are added to a solution of 1.6 g of benzyl (3S,4R,5R)-3-methoxycarbonyl methoxy-4-[4-(3-methoxypropoxy)phenyl]-5-[4-(3-methoxy-propyl)-3,4-dihydro-2H-benzo[1,4]oxazin-6-ylmethoxy]piperidine-1-carboxylate in 6 ml of tetrahydrofuran and the mixture is stirred at room temperature for 2 hours. The reaction mixture is adjusted to pH 2 with 2M HCl. The resulting mixture is extracted twice with 150 ml of ethyl acetate each time. The combined organic phases are washed with brine, dried with sodium sulfate and evaporated. The title compound is obtained as a colourless oil and employed without further purification in the next stage. Rt=4.78 (Gradient I).

### c) Benzyl (3S,4R,5R)-3-methoxycarbonyl methoxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate

0.29 g of sodium hydride (60% dispersion in oil) is added to a solution of 3.5 g of benzyl (3S,4S,5R)-3-hydroxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate (Example 20b) and the mixture is stirred at room temperature for 1 hour. It is then cooled to −5° C., and 1.30 g of methyl bromo-acetate are added dropwise over the course of one hour. The reaction mixture is stirred at −5° C. for 3 hours and then warmed to room temperature. It is then diluted with tert-butyl methyl ether and poured into 0.5M HCl. The resulting mixture is extracted 3 times with tert-butyl methyl ether. The combined organic phases are washed with brine, dried with sodium sulfate and evaporated. The title compound is obtained as a yellow oil from the residue by flash chromatography (SiO₂ 60F). Rf=0.22 (EtOAc-heptane 2:1); Rt=5.26 (Gradient I).

The following compounds are prepared in an analogous manner to the process described in Example 39:

### Examples

### 40 N-Ethyl-2-{(3S,4R,5R)-4-[4-(3-methoxypropoxy)phenyl]-5-[1,4-(3-methoxypropyl)-3,4-dihydro-2H-benzo[1,4]oxazin-6-ylmethoxy]piperidin-3-yloxy}-N-methylacetamide

### 41 2-{(3S,4R,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}-N-methyl-N-propylacetamide

### 46 2-{(3S,4R,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}-N-propylacetamide

### 47 2-{(3S,4R,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}-1-pyrrolidin-1-ylethanone

### 110 N-Ethyl-2-{(3S,4R,5R)-4-[4-(4-methoxybutoxy)phenyl]-5-[4-(3-methoxypropyl)-3,4-dihydro-2H-benzo[1,4]oxazin-6-ylmethoxy]piperidin-3-yloxy}-N-methylacetamide

starting from benzyl (3S,4S,5R)-3-hydroxy-4-[4-(4-methoxybutoxy)phenyl]-5-[4-(3-methoxy-propyl)-3,4-dihydro-2H-benzo[1,4]oxazin-6-ylmethoxy]piperidine-1-carboxylate (Example 78a)

### 111 N,N-Diethyl-2-{(3S,4R,5R)-4-[4-(4-methoxybutoxy)phenyl]-5-[4-(3-methoxypropyl)-3,4-dihydro-2H-benzo[1,4]oxazin-6-ylmethoxy]piperidin-3-yloxy}acetamide

starting from benzyl (3S,4S,5R)-3-hydroxy-4-[4-(4-methoxybutoxy)phenyl]-5-[4-(3-methoxy-propyl)-3,4-dihydro-2H-benzo[1,4]oxazin-6-ylmethoxy]piperidine-1-carboxylate (Example 78a)

### 112 2-{(3S,4R,5R)-4-[4-(4-Methoxybutoxy)phenyl]-5-[4-(3-methoxypropyl)-3,4-dihydro-2H-benzo[1,4]oxazin-6-ylmethoxy]piperidin-3-yloxy}-1-pyrrolidin-1-yl-ethanone

starting from benzyl (3S,4S,5R)-3-hydroxy-4-[4-(4-methoxybutoxy)phenyl]-5-[4-(3-methoxy-propyl)-3,4-dihydro-2H-benzo[1,4]oxazin-6-ylmethoxy]piperidine-1-carboxylate (Example 78a)

### Example 42

### 6-[(3R,4R,5S)-4-[4-(3-Methoxypropoxy)phenyl]-5-(3-methyl-3H-imidazol-4-yl-methoxy)-piperidin-3-yloxymethyl]-4-(3-methoxypropyl)-3,4-dihydro-2H-benzo[i 4]oxazine

The title compound is obtained from 0.199 g of benzyl (3R,4R,5S)-4-[4-(3-methoxypropoxy)-phenyl]-3-[4-(3-methoxypropyl)-3,4-dihydro-2H-benzo[1,4]oxazin-6-ylmethoxy]-5-(3-methyl-3H-imidazol-4-ylmethoxy)piperidine-1-carboxylate in analogy to method B.

The starting material is prepared as follows:

### a) Benzyl (3R,4R,5S)-4-[4-(3-methoxypropoxy)phenyl]-3-[4-(3-methoxypropyl)-3,4-dihydro-2H-benzo[i 4]oxazin-6-ylmethoxy]-5(3-methyl-3H-imidazol-4-ylmethoxy)piperidine-1-carboxylate

0.123 g of sodium hydride (60% dispersion in oil) is added to a solution of 0.778 g of benzyl (3S,4S,5R)-hydroxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate (Example 20b) and 0.269 g of 5-chloromethyl-1-methyl-1H-imidazole hydrochloride [90773-41-4] in 5 ml of N,N-dimethyl-formamide at 0° C. 0.046 g of tetrabutylammonium iodide is added, and the reaction mixture is stirred at room temperature for 18 hours. The reaction mixture is poured into saturated aqueous sodium bicarbonate solution and extracted with tert-butyl methyl ether. The combined organic extracts are washed with brine, dried with sodium sulfate and evaporated. The title compound is obtained as a yellowish oil from the residue by flash chromatography (SiO₂ 60F). Rf=0.20 (dichloromethane-methanol 95:5); Rt=4.51 (Gradient I).

### Example 43

### 6-[(3R,4R,5S)-4-[4-(3-Methoxypropoxy)phenyl]-5-(2-[2,4]triazol-a-yl-ethoxy)piperidin-3-yloxymethyl]-4-(3-methoxypropyl)-3,4-dihydro-2H-benzo[1,4]oxazine

The title compound is prepared from 1.210 g of benzyl (3R,4R,5S)-4-[4-(3-methoxypropoxy)-phenyl]-3-[4-(3-methoxypropyl)-3,4-dihydro-2H-benzo[1,4]oxazin-6-ylmethoxy]-5-(2-triisopropylsilanyloxyethoxy)piperidine-1-carboxylate in analogy to the process described in Example 38.

The starting materials are prepared as follows:

### a) Benzyl (3S,4R,5R)-3-(2-hydroxyethoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate

The title compound is obtained as a pale yellow resin from benzyl (3R,4R,5S)-4-[4-(3-methoxypropoxy)phenyl]-3-[4-(3-methoxypropyl)-3,4-dihydro-2H-benzo[1,4]oxazin-6-ylmethoxy]-5-(2-trisopropylsilanyloxyethoxy)piperidine-1-carboxylate in analogy to method J. Rf=0.14 (EtOAc-heptane 3:1); Rt=4.87 (Gradient I).

### b) Benzyl (3R,4R,5S)-4-[4-(3-methoxypropoxy)phenyl]-3-[4-(3-methoxypropyl)-3,4-dihydro-2H-benzo[1,4]oxazin-6-ylmethoxy]-5-(2-trisopropylsilanyloxyethoxy)piperidine-1-carboxylate

0.164 g of sodium hydride (60% dispersion in oil) is added to a solution of 2.000 g of benzyl (3S,4S,5R)-3-hydroxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate (Example 20b) in 15 ml of dry N,N-dimethylformamide. The reaction mixture is stirred at room temperature for 10 minutes and 2.276 g of (2-iodoethoxy)triisopropylsilane [93550-77-7] are added. The reaction mixture is stirred at room temperature for 18 hours, poured into saturated aqueous sodium bicarbonate solution and extracted with tert-butyl methyl ether. The combined organic phases are washed with brine, dried with sodium sulfate and evaporated. The title compound is obtained as an orange oil from the residue by flash chromatography (SiO₂ 60F). Rf=0.61 (EtOAc-heptane 2:1).

### Example 45

### 4-(2-{(3S,4S,5R)-4-(4-Methoxyphenyl)-5-[4-(3-methoxypropyl)-3,4-dihydro-2H-benzo[1,4]oxazin-6-ylmethoxy]piperidin-3-yloxy}ethyl)tetrahydropyran-4-ol

0.0594 g of lithium aluminium hydride is added to a solution of 0.288 g of 6-[(3R,4R,5S)-5-(1,6-dioxaspiro[2,5]oct-2-ylmethoxy)-4-(4-methoxyphenyl)piperidin-3-yloxymethyl]-4-(3-methoxypropyl)-3,4-dihydro-2H-benzo[1,4]oxazine in 28 ml of diethyl ether. After 45 minutes at room temperature, the reaction mixture is diluted with 100 ml of tert-butyl methyl ether and cautiously quenched with 30 ml of 0.5N NaOH. The aqueous phase is again extracted with 100 ml of tert-butyl methyl ether. The combined organic phases are dried with sodium sulfate and evaporated. The title compound is obtained from the residue by flash chromatography (SiO₂ 60F).

The starting materials are prepared as follows:

### a) 6-[(3R,4R,5S)-5-(1,6-Dioxaspiro[2.5]oct-2-ylmethoxy)-4-(4-methoxyphenyl)piperidin-3-yloxymethyl]-4-(3-methoxypropyl)-3,4-dihydro-2H-benzo[1,4]oxazine

0.387 g of benzyl (3S,4R,5R)-3-(1,6-dioxaspiro[2.5]oct-2-ylmethoxy)-4-(4-methoxyphenyl)-5-[4-(3-methoxypropyl)-3,4-dihydro-2H-benzo[1,4]oxazin-6-ylmethoxy]piperidine-1-carboxylate are reacted in analogy to method B. The title compound is obtained as a brown oil. Rt=3.48 (Gradient I).

### b) Benzyl (3S,4R,5R)-3-(1,6-dioxaspiro[2.5]oct-2-ylmethoxy)-4-(4-methoxyphenyl)-5-[4-(3-methoxypropyl)-3,4-dihydro-2H-benzo[1,4]oxazin-6-ylmethoxy]piperidine-1-carboxylate

0.548 g of benzyl (3S,4R,5R)-3-(1,6-dioxaspiro[2.5]oct-2-ylmethoxy)-4-(4-methoxyphenyl)-5-[4-(3-methoxypropyl)-3-oxo-3,4-dihydro-2H-benzo[1,4]oxazin-6-ylmethoxy]piperidine-1-carboxylate is reacted in analogy to method K. The title compound is obtained as a yellowish oil. Rf=0.33 (EtOAc-heptane 2:1); Rt=5.11 (Gradient I).

### c) Benzyl (3S,4R,5R)-3-(1,6-dioxaspiro[2.5]oct-2-ylmethoxy)-4-(4-methoxyphenyl)-5-[4-(3-methoxypropyl)-3-oxo-3,4-dihydro-2H-benzo[1,4]oxazin-6-ylmethoxy]piperidine-1-carboxylate

0.236 g of 3-chloroperoxybenzoic acid (70% mCPBA) is added to a solution of 0.61 g of benzyl (3R,4S,5S)-4-(4-methoxyphenyl)-3-[4-(3-methoxypropyl)-3-oxo-3,4-dihydro-2H-benzo[1,4]oxazin-6-ylmethoxy]-5-[2-(tetrahydropyran-4-ylidene)ethoxy]piperidine-1-carboxylate in 5 ml of dichloromethane. After 90 minutes at room temperature, the reaction mixture is diluted with 200 ml of tert-butyl methyl ether. The mixture is washed successively with 15 ml of saturated aqueous sodium carbonate solution, 15 ml of saturated aqueous sodium bicarbonate solution, 30 ml of water and 20 ml of brine. The organic phase is dried with sodium sulfate and evaporated. The title compound is obtained as a yellow oil from the residue by flash chromatography (SiO₂ 60F). Rf=0.18 (EtOAc-heptane 2:1); Rt=4.78 (Gradient I).

### d) Benzyl (3R,4S,5S)-4-(4-methoxyphenyl)-3-[4-(3-methoxypropyl)-3-oxo-3,4-dihydro-2H-benzo[1,4]oxazin-6-ylmethoxy]-5-[2-(tetrahydropyran-4-ylidene)ethoxy]piperidine-1-carboxylate

0.057 g of sodium hydride (60% dispersion in oil) is added to a stirred solution of 0.56 g of benzyl (3S,4S,5R)-3-hydroxy-4-(4-methoxyphenyl)-5-[4-(3-methoxypropyl)-3-oxo-3,4-dihydro-2H-benzo[1,4]oxazin-6-ylmethoxy]piperidine-1-carboxylate and 0.546 g of 4-(2-bromoethylidene)tetrahydropyran [21378-20-1] in 5 ml of tetrahydrofuran. After 2.5 hours at room temperature, the reaction mixture is diluted with 200 ml of tert-butyl methyl ether and washed successively with 15 ml of saturated aqueous sodium bicarbonate solution, 10 ml of water and 10 ml of brine. The organic phase is dried with sodium sulfate and evaporated. The title compound is obtained as a yellow oil from the residue by flash chromatography (SiO₂ 60F). Rf=0.20 (EtOAc-heptane 2:1); Rt=5.10 (Gradient I).

### e) Benzyl (3S,4S,5R)-3-hydroxy-4-(4-methoxyphenyl)-5-[4-(3-methoxypropyl)-3-oxo-3,4-dihydro-2H-benzo[1,4]oxazin-6-ylmethoxy]piperidine-1-carboxylate

1.0 g of benzyl (3R,4R,5S)-4-(4-methoxyphenyl)-3-[4-(3-methoxypropyl)-3-oxo-3,4-dihydro-2H-benzo[1,4]oxazin-6-ylmethoxy]-5-triisopropylsilanyloxypiperidine-1-carboxylate is reacted in analogy to Example 1b. The title compound is obtained as a colourless resin. Rf=0.29 (EtOAc-heptane 2:1); Rt=4.36 (Gradient I).

### f) Benzyl (3R,4R,5S)-4-(4-methoxyphenyl)-3-[4-(3-methoxypropyl)-3-oxo-3,4-dihydro-2H-benzo[1,4]oxazin-6-ylmethoxy]-5-triisopropylsilanyloxypiperidine-1-carboxylate

26.59 g of benzyl (3R,4R,5S)-3-hydroxy-4-(4-methoxyphenyl)-5-triisopropylsilanyloxy-piperidine-1-carboxylate (Example 19d) and 17.09 g of 6-chloromethyl-4-(3-methoxypropyl)-4H-benzo[1,4]oxazin-3-one are reacted in analogy to method D. The title compound is obtained as a yellowish resin. Rf=0.18 (EtOAc-heptane 1:2); Rt=6.67 (Gradient I).

### Example 48

### (2-{(3S,4R,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}ethyl)-2-dimethylamine

0.100 g of 4 Å molecular sieve are added to a solution of 0.129 g of benzyl (3R,4R,5S)-4-[4-(3-methoxypropoxy)phenyl]-3-[4-(3-methoxypropyl)-3,4-dihydro-2H-benzo[1,4]oxazin-6-ylmethoxy]-5-(2-oxoethoxy)piperidine-1-carboxylate in 1.5 ml of tetrahydrofuran. A solution of 0.030 g of dimethylamine in 1 ml of tetrahydrofuran is added dropwise, and the reaction mixture is stirred at 20° C. for 1 hour. The solid is filtered off through Hyflo and the filter cake is washed with tetrahydrofuran. The filtrate is degassed with argon for 15 minutes and, after addition of 0.025 g of 10% Pd/C, hydrogenated under atmospheric pressure at 20° C. for 5 hours. The catalyst is filtered off and the filtrate is mixed with a further 0.025 g of Pd/C and 0.155 ml of 2M HCl and hydrogenated under atmospheric pressure at 20° C. for 12 hours. The catalyst is filtered off through Hyflo and the filtrate is evaporated. The title compound is obtained as a brown resin from the residue by flash chromatography (SiO₂ 60F).

The starting material is prepared as follows:

### a) Benzyl (3R,4R,5S)-4-[4-(3-methoxypropoxy)phenyl]-3-[4-(3-methoxypropyl)-3,4-dihydro-2H-benzo[1,4]oxazin-6-ylmethoxy]-5-(2-oxoethoxy)piperidine-1-carboxylate

The title compound is prepared from 1.121 g of benzyl (3R,4R,5S)-4-[4-(3-methoxypropoxy)-phenyl]-3-[4-(3-methoxypropyl)-3,4-dihydro-2H-benzo[1,4]oxazin-6-ylmethoxy]-5-(2-triisopropylsilanyloxyethoxy)piperidine-1-carboxylate (Example 43a) in analogy to the process described in Example 37a-b. The title compound is obtained as a brown resin from the residue by flash chromatography (SiO₂ 60F). Rf=0.18 (EtOAc-heptane 3:1); Rt=4.74 (Gradient I).

### Example 49

### 6-{(3R,4S,5S)-4-(4-Methoxyphenyl)-5-[2-(4-methoxytetrahydropyran-4-yl)ethoxy]piperidin-3-yloxymethyl}-4-(3-methoxypropyl)-3,4-dihydro-2H-benzo[1,4]oxazine

The title compound is prepared from 0.169 g of benzyl (3R,4S,5S)-4-(4-methoxyphenyl)-3-[4-(3-methoxypropyl)-3,4-dihydro-2H-benzo[1,4]oxazin-6-ylmethoxy]-5-[2-(4-methoxy-tetra-hydropyran-4-yl)ethoxy]piperidine-1-carboxylate in analogy to method B.

The starting materials are prepared as follows:

### a) Benzyl (3R,4S,5S)-4-(4-methoxyphenyl)-3-[4-(3-methoxypropyl)-3,4-dihydro-2H-benzo[1,4]oxazin-6-ylmethoxy]-5-[2-(4-methoxytetrahydropyran-4-yl)ethoxy]piperidine-1-carboxylate

0.014 g of sodium hydride (60% dispersion in oil) is added to a solution of 0.166 g of benzyl (3S,4S,5R)-3-[2-(4-hydroxytetrahydropyran-4-yl)ethoxy]-4-(4-methoxyphenyl)-5-[4-(3-methoxypropyl)-3,4-dihydro-2H-benzo[1,4]oxazin-6-ylmethoxy]piperidine-1-carboxylate and 0.074 ml of methyl iodide in 2.5 ml of 4:1 tetrahydrofuran-N,N-dimethylformamide. After 3 hours at room temperature, a further 0.014 g of sodium hydride (60% dispersion in oil) and 0.074 ml of methyl iodide are added. After 14 hours at room temperature, the reaction mixture is diluted with tert-butyl methyl ether and washed with saturated aqueous sodium bicarbonate solution. The aqueous phase is extracted with tert-butyl methyl ether. The combined organic phases are washed with water and brine, dried with sodium sulfate and evaporated. The crude title compound is obtained as a cloudy oil from the residue. Rf=0.75 (EtOAc); Rt=5.31 (Gradient I).

### b) Benzyl (3S,4S,5R)-3-[2-(4-hydroxytetrahydropyran-4-yl)ethoxy]-4-(4-methoxyphenyl)-5-[4-(3-methoxypropyl)-3,4-dihydro-2H-benzo[1,4]oxazin-6-ylmethoxy]piperidine-1-carboxylate

0.135 g of 4-(2-{(3S,4S,5R)-4-(4-methoxyphenyl)-5-[4-(3-methoxypropyl)-3,4-dihydro-2H-benzo[1,4]oxazin-6-ylmethoxy]piperidin-3-yloxy}ethyl)tetrahydropyran-4-ol (Example 45) is reacted in analogy to Example 11h. The title compound is obtained as a brown oil. Rf=0.43 (EtOAc); Rt=4.87 (Gradient I).

### Example 50

### 6-[(3R,4R,5S)-4-[4-(3-Methoxypropoxy)phenyl]-5-(2-morpholin-4-ylethoxy)piperidin-3-yloxymethyl]-4-(3-methoxypropyl)-3,4-dihydro-2H-benzo[1,4]oxazine

The title compound is prepared from 0.191 g of benzyl (3R,4R,5S)-4-[4-(3-methoxypropoxy)-phenyl]-3-[4-(3-methoxypropyl)-3,4-dihydro-2H-benzo[1,4]oxazin-6-ylmethoxy]-5-(2-morpholin-4-yl-ethoxy)piperidine-1-carboxylate in analogy to method B.

The starting materials are prepared as follows:

### a) Benzyl (3R,4R,5S)-4-[4-(3-methoxypropoxy)phenyl]-3-[4-(3-methoxypropyl)-3,4-dihydro-2H-benzo[1,4]oxazin-6-ylmethoxy]-5-(2-morpholin-4-ylethoxy)piperidine-1-carboxylate

0.279 g of benzyl (3R,4R,5S)-4-[4-(3-methoxypropoxy)phenyl]-3-[4-(3-methoxypropyl)-3,4-dihydro-2H-benzo[1,4]oxazin-6-ylmethoxy]-5-(2-morpholin-4-yl-2-oxoethoxy)piperidine-1-carboxylate is reacted in analogy to method K. The methanolysis is carried out with 7 ml of methanol at 65° C. for 24 hours. The title compound is obtained as a colourless oil. Rt=4.46 (Gradient I).

### b) Benzyl (3R,4R,5S)-4-[4-(3-methoxypropoxy)phenyl]-3-[4-(3-methoxypropyl)-3,4-dihydro-2H-benzo[1,4]oxazin-6-ylmethoxy]-5-(2-morpholin-4-yl-2-oxoethoxy)piperidine-1-carboxylate

0.276 g of benzyl (3S,4R,5R)-3-carboxymethoxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate (Example 39b) and 0.045 ml of morpholine are reacted in analogy to Example 39a. The title compound is obtained as a yellowish oil. Rt=4.87 (Gradient I).

The following compound is prepared in an analogous manner to the process described in Example 50:

### Example 54

### 6-[(3R,4R,5S)-4-[4-(3-Methoxypropoxy)phenyl]-5-(2-pyrrolidin-1-yl-ethoxy)piperidin-3-yloxymethyl]-4-(3-methoxypropyl)-3,4-dihydro-2H-benzo[1,4]oxazine

### Example 51

### (3S,4S,5R)-4-[4-(3-Methoxypropoxy)phenyl]-5-[4-(3-methoxypropyl)-3,4-dihydro-2H-benzo[1,4]oxazin-6-ylmethoxy]piperidin-3-ol

The title compound is prepared from 0.360 g of benzyl (3S,4S,5R)-3-hydroxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate (Example 20b) in analogy to method B.

### Example 52

### 6-{(3R,4S,5S)-4-[4-(3-Methoxypropoxy)phenyl]-5-propoxypiperidin-3-yloxymethyl}-4-(3-methoxypropyl)-3,4-dihydro-2H-benzo[1,4]oxazine

The title compound is prepared from 0.255 g of benzyl (3S,4S,5R)-3-allyloxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate in analogy to method B.

The starting material is prepared as follows:

### a) Benzyl (3S,4S,5R)-3-allyloxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate

The title compound is obtained as a yellow resin from 0.400 g of benzyl (3S,4S,5R)-3-hydroxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate (Example 20b) and 0.11 ml of allyl bromide in analogy to method D. Rf=0.13 (EtOAc-heptane 1:1); Rt=5.64 (Gradient I).

### Example 53

### 2-{(3S,4R,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}-N,N-dimethylacetamide

The title compound is prepared from 0.262 g of benzyl (3S,4R,5R)-3-dimethylcarbamoyl-methoxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate in analogy to method B.

The starting material is prepared as follows:

### a) Benzyl (3S,4R,5R)-3-dimethylcarbamoyl methoxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate

A solution of 0.266 g of benzyl (3S,4R,5R)-3-methoxycarbonylmethoxy-4-[4-(3-methoxy-propoxy)phenyl]-5-[4-(3-methoxypropyl)-3,4-dihydro-2H-benzo[1,4]oxazin-6-ylmethoxy]-piperidine-1-carboxylate (Example 39c) in 14 ml of dimethylamine (33% in ethanol) is stirred at 60° C. for 24 hours. The reaction mixture is evaporated. The crude title compound is obtained as a yellow oil from the residue. Rt=4.88 (Gradient I).

### Example 55

### 2-{(3S,4R,5R)-4-(4-Methoxyphenyl)-5-[4-(3-methoxypropyl)-3,4-dihydro-2H-benzo[1,4]oxazin-6-ylmethoxy]piperidin-3-yloxy}-1-((S)-3-methylmorpholin-4-yl)ethanone

The title compound is prepared from 0.065 g of 2-[(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)-piperidin-3-yloxy]-1-((S)-3-methylmorpholin-4-yl)ethanone in analogy to method L.

The starting materials are prepared as follows:

### a) 2-[(3S,4R,5R)-4-(4-Methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]-1-((S)-3-methyl-morpholin-4-yl)ethanone

0.389 ml of propanephosphonic anhydride [68957-94-8, T3P] (50% in ethyl acetate) is added to a solution of 0.40 g of [(3S,4R,5R)-4-(4-methoxyphenyl)-5-[4-(3-methoxypropyl)-3,4-dihydro-2H-benzo[1,4]oxazin-6-ylmethoxy]-1-(toluene-4-su lfonyl)piperidin-3-yloxy]acetic acid, 0.0671 g of (S)-3-methylmorpholine [350595-57-2] and 0.385 ml of triethylamine in 8 ml of dichloromethane at 0° C., and the mixture is stirred at room temperature for 3 hours. The reaction mixture is diluted with dichloromethane, and 0.2M HCl is added. The phases are separated and the aqueous phase is extracted twice more with dichloromethane. The combined organic phases are washed with brine, dried with sodium sulfate and evaporated. The title compound is obtained as a yellow oil from the residue by flash chromatography (SiO₂ 60F). Rf=0.53 (EtOAc); Rt=4.93 (Gradient I).

### b) [(3S,4R,5R)-4-(4-Methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]acetic Acid

5 ml of a 1.5M aqueous lithium hydroxide solution are added to a solution of 0.75 g of methyl [(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]acetate (Example 28b) in 5 ml of tetrahydrofuran, and the mixture is stirred at room temperature for 30 minutes. The reaction mixture is adjusted to pH 2 with 1 M HCl. The resulting mixture is extracted twice with 80 ml of ethyl acetate each time. The combined organic phases are washed with brine, dried with sodium sulfate and evaporated. The title compound is obtained as a yellow oil and is used without further purification in the next stage. Rf=0.15 (EtOAc); Rt=4.70 (Gradient I).

The following compounds are prepared in an analogous manner to the process described in Example 55:

### Examples

### 56 2-{(3S,4R,5R)-4-(4-Methoxyphenyl)-5-[4-(3-methoxypropyl)-3,4-dihydro-2H-benzo[1,4]oxazin-6-ylmethoxy]piperidin-3-yloxy}-1-((S)-2-methylpiperidin-1-yl)-ethanone

### 57 1-((3S,5S)-3,5-Dimethylmorpholin-4-yl)-2-{(3S,4R,5R)-4-(4-methoxyphenyl)-5-[4-(3-methoxypropyl)-3,4-dihydro-2H-benzo[1,4]oxazin-6-ylmethoxy]piperidin-3-yloxy}-ethanone

### 58 2-{(3S,4R,5R)-4-(4-Methoxyphenyl)-5-[4-(3-methoxypropyl)-3,4-dihydro-2H-benzo[1,4]oxazin-6-ylmethoxy]piperidin-3-yloxy}-1-((R)-3-methylmorpholin-4-yl)-ethanone

### 61 1-((3S,5R)-3,5-Dimethylmorpholin-4-yl)-2-{(3S,4R,5R)-4-(4-methoxyphenyl)-5-[4-(3-methoxypropyl)-3,4-dihydro-2H-benzo[1,4]oxazin-6-ylmethoxy]piperidin-3-yloxy}-ethanone

### 75 1-((2S,6R)-2,6-Dimethylpiperidin-1-yl)-2-{(3S,4R,5R)-4-(4-methoxyphenyl)-5-[4-(3-methoxypropyl)-3,4-dihydro-2H-benzo[1,4]oxazin-6-ylmethoxy]piperidin-3-yloxy}-ethanone

### 79 2-{(3S,4R,5R)-4-(4-Methoxyphenyl)-5-[4-(3-methoxypropyl)-3,4-dihydro-2H-benzo[1,4]oxazin-6-ylmethoxy]piperidin-3-yloxy}-1-((R)-2-methylpiperidin-1-yl)-ethanone

### 99 2-{(3S,4R,5R)-4-(4-Methoxyphenyl)-5-[4-(3-methoxypropyl)-3,4-dihydro-2H-benzo[1,4]oxazin-6-ylmethoxy]piperidin-3-yloxy}-1-((R)-2-methylpyrrolidin-1-yl)-ethanone

### 100 N,N-Diisopropyl-2-{(3S,4R,5R)-4-(4-methoxyphenyl)-5-[4-(3-methoxypropyl)-3,4-dihydro-2H-benzo[1,4]oxazin-6-ylmethoxy]piperidin-3-yloxy}acetamide

### 101 2-{(3S,4R,5R)-4-(4-Methoxyphenyl)-5-[4-(3-methoxypropyl)-3,4-dihydro-2H-benzo[1,4]oxazin-6-ylmethoxy]piperidin-3-yloxy}-1-piperidin-1-ylethanone

### Example 59

### 6-{(3R,4R,5S)-4-(4-Methoxyphenyl)-5-[2-((S)-3-methylmorpholin-4-yl)ethoxy]piperidin-3-yloxymethyl}-4-(3-methoxypropyl)-3,4-dihydro-2H-benzo[1,4]oxazine

The title compound is prepared from 0.439 g of 6-[(3R,4R,5S)-4-(4-methoxyphenyl)-5-[2-((S)-3-methylmorpholin-4-yl)ethoxy]-1-(toluene-4-sulfonyl)piperidin-3-yloxymethyl]-4-(3-methoxy-propyl)-3,4-dihydro-2H-benzo[1,4]oxazine in analogy to method L.

The starting materials are prepared as follows:

### a) 6-[(3R,4R,5S)-4-(4-Methoxyphenyl)-5-[2-((S)-3-methylmorpholin-4-yl)ethoxy]-1-(toluene-4-sulfonyl)piperidin-3-yloxymethyl]-4-(3-methoxypropyl)-3,4-dihydro-2H-benzo[1,4]oxazine

2 ml of borane-tetrahydrofuran complex solution (1 M in tetrahydrofuran) are added to a solution of 0.493 g of 2-[(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]-1-((S)-3-methylmorpholin-4-yl)ethanone in 20 ml of tetrahydrofuran, and the mixture is stirred at 55° C. for 16 hours. The reaction mixture is then mixed with 10 ml of methanol and heated at 65° C. for 2 hours. The solution is evaporated in vacuo, and the title compound is obtained as a yellow oil from the residue by flash chromatography (SiO₂ 60F). Rf=0.07 (EtOAc); Rt=4.52 (Gradient I).

### b) 2-[(3S,4R,5R)-4-(4-Methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]-1-((S)-3-methyl-morpholin-4-yl)ethanone

0.389 ml of propanephosphonic anhydride [68957-94-8, T3P] (50% in ethyl acetate) is added to a solution of 0.40 g of [(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]acetic acid, 0.0671 g of (3S)-3-methylmorpholine [350595-57-2] and 0.385 ml of triethylamine in 8 ml of dichloromethane at 0° C., and the mixture is stirred at room temperature for 3 hours. The reaction mixture is diluted with dichloromethane, and 0.2M HCl is added. The phases are separated and the aqueous phase is extracted twice more with dichloromethane. The combined organic phases are washed with brine, dried with sodium sulfate and evaporated. The title compound is obtained as a yellow oil from the residue by flash chromatography (SiO₂ 60F). Rf=0.53 (EtOAc); Rt=4.93 (Gradient I).

### c) [(3S,4R,5R)-4-(4-Methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]acetic Acid

5 ml of a 1.5M aqueous lithium hydroxide solution are added to a solution of 0.75 g of methyl [(3S,4R,5R)-4-(4-methoxyphenyl)-5-[4-(3-methoxypropyl)-3,4-dihydro-2H-benzo[1,4]oxazin-6-ylmethoxy]-1-(toluene-4-sulfonyl)piperidin-3-yloxy]acetate (Example 28b) in 5 ml of tetrahydrofuran, and the mixture is stirred at room temperature for 30 minutes. The reaction mixture is adjusted to pH 2 with 1 M HCl. The resulting mixture is extracted twice with 80 ml of ethyl acetate each time. The combined organic phases are washed with brine, dried with sodium sulfate and evaporated. The title compound is obtained as a yellow oil and is used without further purification in the next stage. Rf=0.15 (EtOAc); Rt=4.70 (Gradient I).

The following compounds are prepared in an analogous manner to the process described in Example 59:

### Examples

### 60 6-{(3R,4R,5S)-4-(4-Methoxyphenyl)-5-[2-((R)-3-methylmorpholin-4-yl)ethoxy]-Piperidin-3-yloxymethyl}-4-(3-methoxypropyl)-3,4-dihydro-2H-benzo[1,4]oxazine

### 62 6-{(3R,4R,5S)-4-(4-Methoxyphenyl)-5-[2-((S)-2-methylpiperidin-1-yl)ethoxy]piperidin-3-yloxymethyl}-4-(3-methoxypropyl)-3,4-dihydro-2H-benzo[1,4]oxazine

### 63 6-[(3R,4R,5S)-5-[2-((3S,5S)-3,5-Dimethylmorpholin-4-yl)ethoxy]-4-(4-methoxy-phenyl)piperidin-3-yloxymethyl]-4-(3-methoxypropyl)-3,4-dihydro-2H-benzo[1,4]oxazine

### 68 6-[(3R,4R,5S)-5-[2-((3R,5S)-3,5-Dimethylmorpholin-4-yl)ethoxy]-4-(4-methoxy-phenyl)piperidin-3-yloxymethyl]-4-(3-methoxypropyl)-3,4-dihydro-2H-benzo[1,4]oxazine

### 76 6-[(3R,4R,5S)-5-[2-((2S,6R)-2,6-Dimethylpiperidin-1-yl)ethoxy]-4-(4-methoxyphenyl)-piperidin-3-yloxymethyl]-4-(3-methoxypropyl)-3,4-dihydro-2H-benzo[1,4]oxazine

### Example 64

### ((R)-2-{(3S,4R,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}-1-methylethyl)dimethylamine

The title compound is prepared from 0.118 g of benzyl (3S,4R,5R)-3-((R)-2-dimethylamino-propoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate in analogy to method B.

The starting materials are prepared as follows:

### a) Benzyl (3S,4R,5R)-3-((R)-2-dimethylaminopropoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate

A solution of 0.177 g of benzyl (3S,4R,5R)-3-((S)-2-methanesulfonyloxypropoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate in 5 ml of dimethylamine (33% in ethanol) is stirred at 50° C. for 20 hours and then evaporated. The residue is diluted with 100 ml of tert-butyl methyl ether and washed with 20 ml of saturated aqueous sodium bicarbonate solution. The aqueous phase is then extracted with 100 ml of tert-butyl methyl ether. The combined organic phases are dried with sodium sulfate and evaporated. The crude title compound is obtained as a yellow oil from the residue. Rt=4.70 (Gradient I).

### b) Benzyl (3S,4R,5R)-3-((S)-2-methanesulfonyloxypropoxy)-4-[4-(3-methoxypropoxy)-phenyl]-5-[4-(3-methoxypropyl)-3,4-dihydro-2H-benzo[1,4]oxazin-6-ylmethoxy]piperidine-1-carboxylate

0.034 ml of methanesulfonyl chloride is added to a solution of 0.27 g of benzyl (3S,4R,5R)-3-((S)-2-hydroxypropoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate and 0.066 ml of triethylamine in 5 ml of dichloromethane at 0° C. After 1 hour at 0° C., a further 0.005 ml of methanesulfonyl chloride and 0.012 ml of triethylamine are added to the reaction solution. After 6 hours at room temperature, the reaction mixture is diluted with 200 ml of tert-butyl methyl ether and washed successively with 20 ml of 0.1N HCl, 30 ml of saturated aqueous sodium bicarbonate solution, 20 ml of water and 10 ml of brine. The organic phase is dried with sodium sulfate and evaporated. The crude title compound is obtained as a yellow oil from the residue. Rf=0.30 (EtOAc-heptane 2:1); Rt=5.27 (Gradient I).

### c) Benzyl (3S,4R,5R)-3-((S)-2-hydroxypropoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate

0.347 g of (S)-1-{(3S,4R,5R)-4-[4-(3-methoxypropoxy)phenyl]-5-[4-(3-methoxypropyl)-3,4-dihydro-2H-benzo[1,4]oxazin-6-ylmethoxy]piperidin-3-yloxy}propan-2-ol (Example 44) are reacted in analogy to Example 11h. The title compound is obtained as a colourless resin. Rf=0.21 (EtOAc-heptane 2:1); Rt=5.03 (Gradient I).

The following compound is prepared in an analogous manner to the process described in Example 64:

### Example 65

### ((S)-2-{(3S,4R,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}-1-methylethyl)dimethyl-amine

### Example 66

### (R)-1-{(3S,4R,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}butan-2-ol

The title compound is obtained from 0.280 g of benzyl (3R,4R,5S)-3-((R)-2-hydroxybutoxy)-4-[4-(3-methoxypropoxy)phenyl]-3-[4-(3-methoxypropyl)-3,4-dihydro-2H-benzo[1,4]oxazin-6-ylmethoxy]piperidine-1-carboxylate in analogy to method B.

The starting material is prepared as follows:

### a) Benzyl (3R,4R,5S)-3-((R)-2-hydroxybutoxy)-4-[4-(3-methoxypropoxy)phenyl]-3-[4-(3-methoxypropyl)-3,4-dihydro-2H-benzo[1,4]oxazin-6-ylmethoxy]piperidine-1-carboxylate

0.010 g of copper(I) cyanide are taken up in 5 ml of dry tetrahydrofuran under argon in a heat-dried Schlenk tube. The suspension is cooled to −78° C., and 0.30 ml of methylmagnesium bromide solution (35% in diethyl ether) is added dropwise. A solution of 0.475 g of benzyl (3R,4R,5S)-4-[4-(3-methoxypropoxy)phenyl]-3-[4-(3-methoxypropyl)-3,4-dihydro-2H-benzo[1,4]oxazin-6-ylmethoxy]-5-((R)-oxiranyl methoxy)piperidine-1-carboxylate (Example 36a) in 4 ml of dry tetrahydrofuran is added, and the reaction mixture is stirred at −78° C. for 30 minutes and then thawed to 20° C. over 16 hours. The reaction mixture is poured into saturated aqueous ammonium chloride solution and adjusted to pH 10 with 25% aqueous ammonium hydroxide solution. The mixture is extracted with diethyl ether, and the combined organic extracts are washed with brine, dried with sodium sulfate and evaporated. The title compound is obtained as a colourless oil from the residue by flash chromatography (SiO₂ 60F). Rf=0.15 (EtOAc-heptane 2:1); Rt=5.22 (Gradient I).

The following compounds are prepared in an analogous manner to the process described in Example 66:

### Examples 67

### (R)-1-{(3S,4R,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}butan-2-ol

using toluene-4-sulfonic acid (S)-1-oxiranylmethyl ester [70987-78-9]

### 106 (S)-1-{(3S,4R,5R)-4-[4-(4-Methoxybutoxy)phenyl]-5-[4-(3-methoxypropyl)-3,4-dihydro-2H-benzo[1,4]oxazin-6-ylmethoxy]piperidin-3-yloxy}butan-2-ol

starting from benzyl (3S,4S,5R)-3-hydroxy-4-[4-(4-methoxybutoxy)phenyl]-5-[4-(3-methoxy-propyl)-3,4-dihydro-2H-benzo[1,4]oxazin-6-ylmethoxy]piperidine-1-carboxylate (Example 78a) using toluene-4-sulfonic acid (S)-1-oxiranylmethyl ester [70987-78-9]

### 108 (R)-1-{(3S,4R,5R)-4-[4-(4-Methoxybutoxy)phenyl]-5-[4-(3-methoxypropyl)-3,4-dihydro-2H-benzo[1,4]oxazin-6-ylmethoxy]piperidin-3-yloxy}butan-2-ol

starting from benzyl (3S,4S,5R)-3-hydroxy-4-[4-(4-methoxybutoxy)phenyl]-5-[4-(3-methoxy-propyl)-3,4-dihydro-2H-benzo[1,4]oxazin-6-ylmethoxy]piperidine-1-carboxylate (Example 78a)

### 113 (S)-1-{(3S,4R,5R)-4-[4-(4-Methoxybutoxy)phenyl]-5-[4-(3-methoxypropyl)-3,4-dihydro-2H-benzo[1,4]oxazin-6-ylmethoxy]piperidin-3-yloxy}pentan-2-ol

starting from benzyl (3S,4S,5R)-3-hydroxy-4-[4-(4-methoxybutoxy)phenyl]-5-[4-(3-methoxy-propyl)-3,4-dihydro-2H-benzo[1,4]oxazin-6-ylmethoxy]piperidine-1-carboxylate (Example 78a) using ethylmagnesium bromide solution (1 M in tetrahydrofuran) using toluene-4-sulfonic acid (S)-1-oxiranylmethyl ester [70987-78-9]

### 114 (R)-1-{(3S,4R,5R)-4-[4-(4-Methoxybutoxy)phenyl]-5-[4-(3-methoxypropyl)-3,4-dihydro-2H-benzo[1,4]oxazin-6-ylmethoxy]piperidin-3-yloxy}pentan-2-ol

starting from benzyl (3S,4S,5R)-3-hydroxy-4-[4-(4-methoxybutoxy)phenyl]-5-[4-(3-methoxy-propyl)-3,4-dihydro-2H-benzo[1,4]oxazin-6-ylmethoxy]piperidine-1-carboxylate (Example 78a) using ethylmagnesium bromide solution (1M in tetrahydrofuran)

### 125 (S)-1-{(3S,4R,5R)-4-[4-(3-Methoxy-propoxy)-phenyl]-5-[4-(3-methoxy-propyl)-3,4-dihydro-2H-benzo[1,4]oxazin-6-ylmethoxy]-piperidin-3-yloxy}-pentan-2-ol

starting from (3S,4S,5R)-3-hydroxy-4-[4-(3-methoxy-propoxy)-phenyl]-5-[4-(3-methoxy-propyl)-3,4-dihydro-2H-benzo[1,4]oxazin-6-ylmethoxy]-piperidin-1-carboxylic acid benzyl ester (Example 20b) using toluene-4-sulfonic acid (S)-1-oxiranylmethyl ester [70987-78-9] and ethylmagnesium bromid solution (1 M in tetrahydrofuran).

### 126 (R)-1-{(3S,4R,5R)-4-[4-(3-Methoxy-propoxy)-phenyl]-5-[4-(3-methoxy-propyl)-3,4-dihydro-2H-benzo[1,4]oxazin-6-ylmethoxy]-piperidin-3-yloxy}-pentan-2-ol

starting from (3S,4S,5R)-3-hydroxy-4-[4-(3-methoxy-propoxy)-phenyl]-5-[4-(3-methoxy-propyl)-3,4-dihydro-2H-benzo[1,4]oxazin-6-ylmethoxy]-piperidin-1-carboxylic acid benzyl ester (Example 20b) using ethylmagnesium bromid solution (1 M in tetrahydrofuran).

### Example 69

### (R)-1-Methoxy-3-[(3S,4R,5R)-5-[4-(3-methoxypropyl)-3,4-dihydro-2H-benzo[1,4]oxazin-6-ylmethoxy]-4-(4-methylsulfanylphenyl)piperidin-3-yloxy]propan-2-ol

3 ml of 40% aqueous potassium hydroxide solution are added to a solution of 0.047 g of benzyl (3S,4R,5R)-3-((R)-2-hydroxy-3-methoxypropoxy)-5-[4-(3-methoxypropyl)-3,4-dihydro-2H-benzo[1,4]oxazin-6-ylmethoxy]-4-(4-methylsulfanylphenyl)piperidine-1-carboxylate in 3 ml of methanol and 1 ml of dioxane. The reaction mixture is heated under reflux for 3 hours. It is then diluted with 40 ml of water and extracted three times with 40 ml of ethyl acetate each time. The combined organic phases are dried with sodium sulfate, filtered and evaporated in vacuo. The title compound is obtained from the residue by flash chromatography (SiO₂ 60F).

The starting materials are prepared as follows:

### a) Benzyl (3S,4R,5R)-3-((R)-2-hydroxy-3-methoxypropoxy)-5-[4-(3-methoxypropyl)-3,4-dihydro-2H-benzo[1,4]oxazin-6-ylmethoxy]-4-(4-methylsulfanylphenyl)piperidine-1-carboxylate

0.068 g of benzyl (3S,4S,5R)-3-hydroxy-5-[4-(3-methoxypropyl)-3,4-dihydro-2H-benzo[1,4]oxazin-6-ylmethoxy]-4-(4-methylsulfanylphenyl)piperidine-1-carboxylate and 0.023 g of S-(+)-glycidyl methyl ether [64491-68-5] are reacted in analogy to method M. The title compound is obtained as a yellow oil. Rf=0.20 (EtOAc-heptane 2:1); Rt=5.04 (Gradient I).

### b) Benzyl (3S,4S,5R)-3-hydroxy-5-[4-(3-methoxypropyl)-3,4-dihydro-2H-benzo[1,4]oxazin-6-ylmethoxy]-4-(4-methylsulfanylphenyl)piperidine-1-carboxylate

0.095 g of benzyl (3R,4R,5S)-3-[4-(3-methoxypropyl)-3,4-dihydro-2H-benzo[1,4]oxazin-6-ylmethoxy]-4-(4-methylsulfanylphenyl)-5-triisopropylsilanyloxypiperidine-1-carboxylate are reacted in analogy to method J. The title compound is obtained as a yellow oil. Rf=0.13 (EtOAc-heptane 1:1); Rt=4.95 (Gradient I).

### c) Benzyl (3R,4R,5S)-3-[4-(3-methoxypropyl)-3,4-dihydro-2H-benzo[1,4]oxazin-6-ylmethoxy]-4-(4-methylsulfanylphenyl)-5-triisopropylsilanyloxypiperidine-1-carboxylate

0.477 g of caesium fluoride is added to a degassed solution of 0.718 g of benzyl (3R,4R,5S)-3-[4-(3-methoxypropyl)-3,4-dihydro-2H-benzo[1,4]oxazin-6-ylmethoxy]-5-triisopropylsilanyl-oxy-4-(4-triisopropylsilanylsulfanylphenyl)piperidine-1-carboxylate in 15 ml of DMF under argon, and the mixture is stirred at room temperature for 2 hours. The mixture is then cooled to −12° C. and, after addition of 0.060 ml of methyl iodide, stirred at this temperature for 3 hours. The reaction mixture is diluted with tert-butyl methyl ether and poured into water. The organic phase is dried with sodium sulfate, filtered and evaporated. The title compound is obtained as a yellow oil from the residue by flash chromatography (SiO₂ 60F). Rf=0.56 (EtOAc-heptane 1:1).

### d) Benzyl (3R,4R,5S)-3-[4-(3-Methoxypropyl)-3,4-dihydro-2H-benzo[1,4]oxazin-6-ylmethoxy]-5-triisopropylsilanyloxy-4-(4-triisopropylsilanylsulfanylphenyl)piperidine-1-carboxylate

0.323 g of sodium tert-butoxide is added to 0.73 ml of triisopropylsilanethiol in 7 ml of toluene in a Schlenk tube at 0° C., and the mixture is stirred at room temperature for 45 minutes. In a second flask, 2.0 g of benzyl (3R,4R,5S)-3-[4-(3-methoxypropyl)-3,4-dihydro-2H-benzo[1,4]oxazin-6-ylmethoxy]-4-(4-trifluoromethanesulfonyloxyphenyl)-5-triisopropylsilanyl-oxypiperidine-1-carboxylate (Example 72c) are dissolved in 7 ml of toluene and, under argon, 0.415 g of palladium(0) tetrakistriphenylphosphine is added. This suspension is added to the above “thiolate” solution which has been preheated to 90° C. and is stirred under argon at 90° C. overnight. The reaction mixture is diluted with tert-butyl methyl ether and poured into water. The organic phase is dried with sodium sulfate, filtered and evaporated. The title compound is obtained as a brown oil from the residue by flash chromatography (SiO₂ 60F). Rf=0.39 (EtOAc-heptane 1:2)

The following compounds are prepared in an analogous manner to the process described in Example 69:

### Examples

### 70 (R)-1-Methoxy-3-{(3S,4R,5R)-4-[4-(2-methoxyethylsulfanyl)phenyl]-5-[4-(3-methoxy-propyl)-3,4-dihydro-2H-benzo[1,4]oxazin-6-ylmethoxy]piperidin-3-yloxy}propan-2-ol

### 71 (R)-1-Methoxy-3-{(3S,4R,5R)-5-[4-(3-methoxypropyl)-3,4-dihydro-2H-benzo[1,4]oxazin-6-ylmethoxy]-4-[4-(3-methoxypropylsulfanyl)phenyl]piperidin-3-yloxy}propan-2-ol

### 103 (R)-1-Methoxy-3-{(3S,4R,5R)-4-[4-(4-methoxybutoxy)phenyl]-5-[4-(3-methoxy-propyl)-3,4-dihydro-2H-benzo[1,4]oxazin-6-ylmethoxy]piperidin-3-yloxy}propan-2-ol

starting from benzyl (3S,4S,5R)-3-hydroxy-4-[4-(4-methoxybutoxy)phenyl]-5-[4-(3-methoxy-propyl)-3,4-dihydro-2H-benzo[1,4]oxazin-6-ylmethoxy]piperidine-1-carboxylate (Example 78a)

### 104 (S)-1-Methoxy-3-{(3S,4R,5R)-4-[4-(4-methoxybutoxy)phenyl]-5-[4-(3-methoxypropyl)-3,4-dihydro-2H-benzo[1,4]oxazin-6-ylmethoxy]piperidin-3-yloxy}propan-2-ol

starting from benzyl (3S,4S,5R)-3-hydroxy-4-[4-(4-methoxybutoxy)phenyl]-5-[4-(3-methoxy-propyl)-3,4-dihydro-2H-benzo[1,4]oxazin-6-ylmethoxy]piperidine-1-carboxylate (Example 78a) using R-(−)-glycidyl methyl ether [64491-70-9].

### Example 72

### 4-{(3S,4S,5R)-3-Hydroxy-5-[4-(3-methoxypropyl)-3,4-dihydro-2H-benzo[1,4]oxazin-6-ylmethoxy]piperidin-4-yl}benzonitrile

The title compound is obtained from 0.0538 g of benzyl (3S,4S,5R)-4-(-cyanophenyl)-3-hydroxy-5-[4-(3-methoxypropyl)-3,4-dihydro-2H-benzo[1,4]oxazin-6-ylmethoxy]piperidine-1-carboxylate in analogy to method B.

The starting materials are prepared as follows:

### a) Benzyl (3S,4S,5R)-4-(4-cyanophenyl)-3-hydroxy-5-[4-(3-methoxypropyl)-3,4-dihydro-2H-benzo[1,4]oxazin-6-ylmethoxy]piperidine-1-carboxylate

A mixture of 1.290 g of benzyl (3S,4S,5R)-3-hydroxy-5-[4-(3-methoxypropyl)-3,4-dihydro-2H-benzo[1,4]oxazin-6-ylmethoxy]-4-(trifluoromethanesulfonyloxyphenyl)piperidine-1-carboxylate, 0.440 g of zinc(II) cyanide and 0.193 g of palladium(0) tetrakistriphenyl-phosphine in 11 ml of dry N,N-dimethylformamide is heated at 120° C. for 16 hours. the reaction mixture is poured into saturated aqueous sodium bicarbonate solution, and the mixture is then extracted with tert-butyl methyl ether. The combined organic phases are washed with brine, dried and evaporated. The title compound is obtained as a colourless resin from the residue by flash chromatography (SiO₂ 60F). Rf=0.11 (EtOAc-heptane 3:2); Rt=4.61 (Gradient I).

### b) Benzyl (3S,4S,5R)-3-hydroxy-5-[4-(3-methoxypropyl)-3,4-dihydro-2H-benzo[1,4]oxazin-6-ylmethoxy]-4-(trifluoromethanesulfonyloxyphenyl)piperidine-1-carboxylate

The title compound is obtained as a pale yellow resin from 2.340 g of benzyl (3S,4R,5R)-3-[4-(3-methoxypropyl)-3,4-dihydro-2H-benzo[1,4]oxazin-6-ylmethoxy]-4-(trifluoromethane-sulfonyloxyphenyl)-5-triisopropylsilanyloxypiperidine-1-carboxylate in analogy to method J. Rf=0.37 (EtOAc-heptane 2:1); Rt=5.20 (Gradient I).

### c) Benzyl (3S,4R,5R)-3-[4-(3-methoxypropyl)-3,4-dihydro-2H-benzo[1,4]oxazin-6-ylmethoxy]-4-(4-trifluoromethanesulfonyloxyphenyl)-5-triisopropylsilanyloxypiperidine-1-carboxylate

0.544 ml of triethylamine is added to a solution of 2.590 g of benzyl (3R,4R,5S)-4-(4-hydroxy-phenyl)-3-[4-(3-methoxypropyl)-3,4-dihydro-2H-benzo[1,4]oxazin-6-ylmethoxy]-5-triisopropylsilanyloxypiperidine-1-carboxylate (Example 11d) and 1.353 g of N-phenyl-bis(trifluoromethanesulfonamide) in 20 ml of dry dichloromethane. The reaction solution is left to stand at room temperature for 3 hours and then evaporated to dryness. The title compound is obtained as a reddish oil from the residue by flash chromatography (SiO₂ 60F). Rf=0.56 (EtOAc-heptane 1:1).

### Example 73

### 4-{(3S,4R,5R)-3((R)-2-Hydroxy-3-methoxypropoxy)-5-[4-(3-methoxypropyl)-3,4-dihydro-2H-benzo[1,4]oxazin-6-ylmethoxy]piperidin-4-yl}benzonitrile

The title compound is obtained from 0.065 g of benzyl (3S,4S,5R)-4-(4-cyanophenyl)-3-((R)-2-hydroxy-3-methoxypropoxy)-5-[4-(3-methoxypropyl)-3,4-dihydro-2H-benzo[1,4]oxazin-6-ylmethoxy]piperidine-1-carboxylate in analogy to method B.

The starting material is prepared as follows:

### a) Benzyl (3S,4S,5R)-4-(4-cyanophenyl)-3-((R)-2-hydroxy-3-methoxypropoxy)-5-[4-(3-methoxypropyl)-3,4-dihydro-2H-benzo[1,4]oxazin-6-ylmethoxy]piperidine-1-carboxylate

The title compound is obtained as a colourless resin from 0.100 g of benzyl (3S,4S,5R)-4-(4-cyanophenyl)-3-hydroxy-5-[4-(3-methoxypropyl)-3,4-dihydro-2H-benzo[1,4]oxazin-6-ylmethoxy]piperidine-1-carboxylate (Example 72a) and 0.061 g of S-(+)-glycidyl methyl ether [64491-68-5] in analogy to method M. Rf=0.16 (EtOAc-heptane 2:1); Rt=4.68 (Gradient I).

The following compound is prepared in an analogous manner to the process described in Example 73:

### Example 74

### 4-{(3S,4R,5R)-3((S)-2-Hydroxy-3-methoxypropoxy)-5-[4-(3-methoxypropyl)-3,4-dihydro-2H-benzo[1,4]oxazin-6-ylmethoxy]piperidin-4-yl}-benzonitrile

### Example 77

### 7-{(3R,4S,5S)-5-Hydroxy-4-[4-(3-methoxypropoxy)phenyl]piperidin-3-yloxymethyl}-3,3-dimethyl-1,3-dihydroindol-2-one

The title compound is prepared from 0.350 g of benzyl (3R,4S,5S)-3-(3,3-dimethyl-2-oxo-2,3-dihydro-1H-indol-7-ylmethoxy)-5-hydroxy-4-[4-(3-methoxypropoxy)phenyl]piperidine-1-carboxylate in analogy to method B.

The starting materials are prepared as follows:

### a) Benzyl (3R,4S,5S)-3-(3,3-dimethyl-2-oxo-2,3-dihydro-1H-indol-7-ylmethoxy)-5-hydroxy-4-[4-(3-methoxypropoxy)phenyl]piperidine-1-carboxylate

40 ml of tetrabutylammonium fluoride (1 M solution in tetrahydrofuran) are added to a solution of 1.78 g of benzyl (3R,4R,5S)-3-[3,3-dimethyl-2-oxo-1-(2-trimethylsilanylethoxymethyl)-2,3-dihydro-1H-indol-7-ylmethoxy]-4-[4-(3-methoxypropoxy)phenyl]-5-triisopropylsilanyloxypiperidine-1-carboxylate in 18 ml of tetrahydrofuran, and the mixture is stirred at the reflux temperature for 4 days. The reaction mixture is poured into ice-water and extracted with tert-butyl methyl ether. The organic phases are washed with water and brine, dried with sodium sulfate and evaporated. The title compound is obtained as a white foam from the residue by flash chromatography (SiO₂ 60F). Rf=0.20 (EtOAc-heptane 1:1); Rt=4.54 (Gradient I).

### b) Benzyl (3R,4R,5S)-3-[3,3-dimethyl-2-oxo-1-(2-trimethylsilanylethoxymethyl)-2,3-dihydro-1H-indol-7-ylmethoxy]-4-[4-(3-methoxypropoxy)phenyl]-5-triisopropylsilanyloxypiperidine-1-carboxylate

2.0 g of benzyl (3R,4R,5S)-3-hydroxy-4-[4-(3-methoxypropoxy)phenyl]-5-triisopropylsilanyloxypiperidine-1-carboxylate (Example 20e) and 1.60 g of 7-bromomethyl-3,3-dimethyl-1-(2-trimethylsilanylethoxymethyl)-1,3-dihydroindol-2-one [985278-97-8] are reacted in analogy to method D. The title compound is obtained as a colourless oil. Rf=0.22 (EtOAc-heptane 1:4).

### Example 78

### (3S,4S,5R)-4-[4-(4-Methoxybutoxy)phenyl]-5-[4-(3-methoxypropyl)-3,4-dihydro-2H-benzo[1,4]oxazin-6-ylmethoxy]piperidin-3-ol

The title compound is prepared from 0.264 g of benzyl (3S,4S,5R)-3-hydroxy-4-[4-(4-methoxybutoxy)phenyl]-5-[4-(3-methoxypropyl)-3,4-dihydro-2H-benzo[1,4]oxazin-6-ylmethoxy]piperidine-1-carboxylate in analogy to method B.

The starting materials are prepared as follows:

### a) Benzyl (3S,4S,5R)-3-hydroxy-4-[4-(4-methoxybutoxy)phenyl]-5-[4-(3-methoxypropyl)-3,4-dihydro-2H-benzo[1,4]oxazin-6-ylmethoxy]piperidine-1-carboxylate

1.11 g of benzyl (3R,4R,5S)-4-[4-(4-methoxybutoxy)phenyl]-3-[4-(3-methoxypropyl)-3,4-dihydro-2H-benzo[1,4]oxazin-6-ylmethoxy]-5-triisopropylsilanyloxypiperidine-1-carboxylate are reacted in analogy to Example 1b. The title compound is obtained as a cloudy white oil. Rf=0.60 (EtOAc); Rt=4.94 (Gradient I).

### b) Benzyl (3R,4R,5S)-4-[4-(4-methoxybutoxy)phenyl]-3-[4-(3-methoxypropyl)-3,4-dihydro-2H-benzo[1,4]oxazin-6-ylmethoxy]-5-triisopropylsilanyloxypiperidine-1-carboxylate

The title compound is prepared from 1.15 g of benzyl (3R,4R,5S)-3-hydroxy-4-(4-hydroxy-phenyl)-5-triisopropylsilanyloxypiperidine-1-carboxylate (Example 11h) and 0.461 g of 1-bromo-3-methoxypropane [4457-67-4] in analogy to the process described in Example 20c-e. Rf=0.19 (EtOAc-heptane 1:1).

### Example 80

### (3S,4S,5R)-5-[4-(3-Methoxypropyl)-3,4-dihydro-2H-benzo[1,4]oxazin-6-ylmethoxy]-4-[4-(3-methylsulfanylpropoxy)phenyl]piperidin-3-ol

5 ml of 40% aqueous potassium hydroxide solution are added to a solution of 0.38 g of benzyl (3S,4S,5R)-3-hydroxy-5-[4-(3-methoxypropyl)-3,4-dihydro-2H-benzo[1,4]oxazin-6-ylmethoxy]-4-[4-(3-methylsulfanylpropoxy)phenyl]piperidine-1-carboxylate in 5 ml of methanol and 2 ml of dioxane. The reaction mixture is heated under reflux for 3 hours. It is then diluted with 40 ml of water and extracted three times with 40 ml of ethyl acetate each time. The combined organic phases are dried with sodium sulfate, filtered and evaporated in vacuo. The title compound is obtained from the residue by flash chromatography (SiO₂ 60F).

The starting materials are prepared as follows:

### a) Benzyl (3S,4S,5R)-3-hydroxy-5-[4-(3-methoxypropyl)-3,4-dihydro-2H-benzo[1,4]oxazin-6-ylmethoxy]-4-[4-(3-methylsulfanylpropoxy)phenyl]piperidine-1-carboxylate

1.2 g of benzyl (3R,4R,5S)-3-[4-(3-methoxypropyl)-3,4-dihydro-2H-benzo[1,4]oxazin-6-ylmethoxy]-4-[4-(3-methylsulfanylpropoxy)phenyl]-5-triisopropylsilanyloxypiperidine-1-carboxylate are reacted in analogy to method J. The title compound is obtained as a colourless resin. Rf=0.18 (EtOAc-heptane 4:1); Rt=5.15 (Gradient I).

### b) Benzyl (3R,4R,5S)-3-[4-(3-methoxypropyl)-3,4-dihydro-2H-benzo[1,4]oxazin-6-ylmethoxy]-4-[4-(3-methylsulfanylpropoxy)phenyl]-5-triisopropylsilanyloxypiperidine-1-carboxylate

3.0 g of benzyl (3R,4R,5S)-4-(4-hydroxyphenyl)-3-[4-(3-methoxypropyl)-3,4-dihydro-2H-benzo[1,4]oxazin-6-ylmethoxy]-5-triisopropylsilanyloxypiperidine-1-carboxylate (Example 11d) and 1.49 g of 3-methylsulfanylpropyl toluene-4-sulfonate [187722-18-5] are reacted in analogy to method G. The title compound is obtained as a yellow oil. Rf=0.18 (EtOAc-heptane 1:2).

### Example 81

### (3S,4S,5R)-4-[4-(4-Methoxybutylsulfanyl)phenyl]-5-[4-(3-methoxypropyl)-3,4-dihydro-2H-benzo[1,4]oxazin-6-ylmethoxy]piperidin-3-ol

A solution of 0.33 g of benzyl (3R,4R,5S)-4-[4-(4-methoxybutylsulfanyl)phenyl]-3-[4-(3-methoxypropyl)-3,4-dihydro-2H-benzo[1,4]oxazin-6-ylmethoxy]-5-triisopropylsilanyl-oxypiperidine-1-carboxylate in 5 ml of dioxane, 6 ml of aqueous 40% potassium hydroxide solution and 6 ml of methanol is stirred at 90° C. for 4 days. The reaction mixture is diluted at room temperature with 50 ml of tert-butyl methyl ether and mixed with 20 ml of water. The aqueous phase is then extracted with 2×50 ml of tert-butyl methyl ether. The combined organic phases are washed with 20 ml of brine, dried with sodium sulfate and evaporated. The title compound is obtained from the residue by flash chromatography (SiO₂ 60F).

The starting materials are prepared as follows:

### a) Benzyl (3R,4R,5S)-4-[4-(4-methoxybutylsulfanyl)phenyl]-3-[4-(3-methoxypropyl)-3,4-dihydro-2H-benzo[1,4]oxazin-6-ylmethoxy]-5-triisopropylsilanyloxypiperidine-1-carboxylate

The title compound is obtained as a colourless oil from benzyl (3R,4R,5S)-4-[4-(4-methoxy-butylsulfanyl)phenyl]-3-[4-(3-methoxypropyl)-3-oxo-3,4-dihydro-2H-benzo[1,4]oxazin-6-ylmethoxy]-5-triisopropylsilanyloxypiperidine-1-carboxylate in analogy to method K (temperature: 45° C.). Rf=0.40 (EtOAc-heptane 1:1).

### b) Benzyl (3R,4R,5S)-4-[4-(4-methoxybutylsulfanyl)phenyl]-3-[4-(3-methoxypropyl)-3-oxo-3,4-dihydro-2H-benzo[1,4]oxazin-6-ylmethoxy]-5-triisopropylsilanyloxypiperidine-1-carboxylate

The title compound is obtained as a yellow oil from benzyl (3R,4R,5S)-3-hydroxy-4-[4-(4-methoxybutylsulfanyl)phenyl]-5-triisopropylsilanyloxypiperidine-1-carboxylate in analogy to Example 16b. Rf=0.45 (EtOAc-heptane 1:1); Rt=7.38 (Gradient I).

### c) Benzyl (3R,4R,5S)-3-hydroxy-4-[4-(4-methoxybutylsulfanyl)phenyl]-5-triisopropylsilanyloxypiperidine-1-carboxylate

The title compound is obtained as a yellow oil from benzyl (3R,4R,5S)-3-hydroxy-5-triisopropylsilanyloxy-4-(4-triisopropylsilanylsulfanylphenyl)piperidine-1-carboxylate and 1-bromo-3-methoxypropane [4457-67-4] in analogy to Example 69c. Rf=0.40 (EtOAc-heptane 1:1); Rt=6.76 (Gradient I).

### d) Benzyl (3R,4R,5S)-3-hydroxy-5-triisopropylsilanyloxy-4-(4-triisopropylsilanylsulfanyl-phenyl)piperidine-1-carboxylate

The title compound is obtained as a red oil from benzyl (3R,4R,5S)-3-hydroxy-4-(4-trifluoromethanesulfonyloxyphenyl)-5-triisopropylsilanyloxypiperidine-1-carboxylate in analogy to Example 69d. Rf=0.07 (EtOAc-heptane 1:4).

### e) Benzyl (3R,4R,5S)-3-hydroxy-4-(4-trifluoromethanesulfonyloxyphenyl)-5-triisopropylsilanyloxypiperidine-1-carboxylate

The title compound is obtained as a yellowish resin from benzyl (3R,4R,5S)-3-hydroxy-4-(4-hydroxyphenyl)-5-triisopropylsilanyloxypiperidine-1-carboxylate (Example 19d) in analogy to Example 72c. Rf=0.58 (EtOAc-heptane 1:1); Rt=6.61 (Gradient I).

The following compound is prepared in an analogous manner to the process described in Example 81:

### Example 82

### (3S,4S,5R)-5-[4-(3-Methoxypropyl)-3,4-dihydro-2H-benzo[1,4]oxazin-6-ylmethoxy]-4-[4-(3-methoxypropylsulfanyl)phenyl]piperidin-3-ol

### Example 83

### (3S,4S,5R)-4-[4-((R)-4-Methoxy-3-methylbutylsulfanyl)phenyl]-5-[4-(3-methoxypropyl)-3,4-dihydro-2H-benzo[1,4]oxazin-6-ylmethoxypiperidein-3-ol

A solution of 0.44 g of methyl (3S,4S,5R)-3-hydroxy-4-[4-((R)-4-methoxy-3-methyl-butylsulfanyl)phenyl]-5-[4-(3-methoxypropyl)-3,4-dihydro-2H-benzo[1,4]oxazin-6-ylmethoxy]-piperidine-1-carboxylate in 1 ml of dioxane, 0.7 ml of aqueous 40% KOH and 1.2 ml of methanol is stirred at 90° C. for 1 hour. The reaction mixture is diluted at room temperature with tert-butyl methyl ether and mixed with water. The aqueous phase is extracted with tert-butyl methyl ether (2×). The combined organic phases are washed with brine, dried with sodium sulfate and evaporated. The title compound is obtained from the residue by flash chromatography (SiO₂ 60F).

The starting materials are prepared as follows:

### a) Methyl (3S,4S,5R)-3-hydroxy-4-[4-((R)-4-methoxy-3-methylbutylsulfanyl)phenyl]-5-[4-(3-methoxypropyl)-3,4-dihydro-2H-benzo[1,4]oxazin-6-ylmethoxy]piperidine-1-carboxylate

0.31 g of methyl (3R,4R,5S)-4-[4-((R)-4-methoxy-3-methylbutylsulfanyl)phenyl]-3-[4-(3-methoxypropyl)-3,4-dihydro-2H-benzo[1,4]oxazin-6-ylmethoxy]-5-triisopropylsilanyloxy-piperidine-1-carboxylate is reacted in analogy to Example 1b. The title compound is obtained as a colourless oil. Rf=0.15 (EtOAc-heptane 2:1); Rt=4.73 (Gradient I).

### b) Methyl (3R,4R,5S)-4-[4-((R)-4-methoxy-3-methylbutylsulfanyl)phenyl]-3-[4-(3-methoxy-propyl)-3,4-dihydro-2H-benzo[1,4]oxazin-6-ylmethoxy]-5-triisopropylsilanyloxypiperidine-1-carboxylate

The title compound is obtained as a colourless oil from 0.5 g of methyl (3R,4R,5S)-3-hydroxy-4-[4-((R)-4-methoxy-3-methyl butylsulfanyl)phenyl]-5-triisopropylsilanyloxypiperidine-1-carboxylate in analogy to the process described in Example 81a-b. Rf=0.53 (EtOAc-heptane 1:1).

### c) Methyl (3R,4R,5S)-3-hydroxy-4-[4-((R)-4-methoxy-3-methylbutylsulfanyl)phenyl]-5-triisopropylsilanyloxypiperidine-1-carboxylate

10.9 g of sodium methoxide are added to a solution of 2.09 g of benzyl (3R,4R,5S)-3-hydroxy-4-{4-[(R)-3-methyl-4-(toluene-4-sulfonyloxy)butylsulfanyl]phenyl}-5-triisopropylsilanyloxypiperidine-1-carboxylate in 11 ml of methanol and 15 ml of tetrahydrofuran. The reaction mixture is stirred at 50° C. until conversion is complete and then diluted with 150 ml of tert-butyl methyl ether at room temperature. The mixture is washed with 40 ml of saturated aqueous sodium bicarbonate solution, 100 ml of water and 30 ml of brine. The organic phase is dried with sodium sulfate and evaporated. The crude title compound is obtained a s yellowish oil from the residue. Rf=0.50 (EtOAc-heptane 1:1); Rt=6.53 (Gradient I).

### d) Benzyl (3R,4R,5S)-3-hydroxy-4-{4-[(R)-3-methyl-4-(toluene-4-sulfonyloxy)butylsulfanyl]-phenyl}-5-triisopropylsilanyloxypiperidine-1-carboxylate

1.5 g of benzyl (3R,4R,5S)-3-hydroxy-5-triisopropylsilanyloxy-4-(4-triisopropylsilanylsulfanyl-phenyl)piperidine-1-carboxylate (Example 81d) and 1.5 g of (R)-2-methylbutane-1,4-diol bistoluenesulfonate [281214-26-4] are reacted in analogy to Example 81c. The title compound is obtained as a yellow oil. Rf=1.6 (EtOAc-heptane 1:2).

### Example 87

### Isopropyl-{(3S,4R,5R)-4-(4-methoxyphenyl)-5-[4-(3-methoxypropyl)-3,4-dihydro-2H-benzo[1,4]oxazin-6-ylmethoxy]piperidin-3-ylmethyl}amine

The title compound is prepared starting from benzyl (3R,4R,5R)-3-(isopropylaminomethyl)-4-(4-methoxyphenyl)-5-[4-(3-methoxypropyl)-3,4-dihydro-2H-benzo[1,4]oxazin-6-ylmethoxy]piperidine-1-carboxylate in analogy to method B.

The starting materials are prepared as follows:

### a) Benzyl (3R,4R,5R)-3-(isopropylaminomethyl)-4-(4-methoxyphenyl)-5-[4-(3-methoxy-propyl)-3,4-dihydro-2H-benzo[1,4]oxazin-6-ylmethoxy]piperidine-1-carboxylate

A solution of 0.50 mmol of benzyl (3R,4R,5S)-4-(4-methoxyphenyl)-3-[4-(3-methoxypropyl)-3,4-dihydro-2H-benzo[1,4]oxazin-6-ylmethoxy]-5-(toluene-4-sulfonyloxymethyl)piperidine-1-carboxylate and 1.0 mmol of isopropylamine in 4 ml of 1-methylpyrrolidin-2-one (NMP) is stirred at 85° C. for 8 hours. The reaction mixture is cooled to room temperature, diluted with water and extracted with dichloromethane (3×). The combined organic phases are washed with brine, dried with sodium sulfate and evaporated. The title compound is obtained as a yellow oil from the residue by flash chromatography (SiO₂ 60F). Rf=0.29 (dichloromethane-methanol-25% conc. ammonia=200:10:1); Rt=4.45 (Gradient I).

### b) Benzyl (3R,4R,5S)-4-(4-methoxyphenyl)-3-[4-(3-methoxypropyl)-3,4-dihydro-2H-benzo[1,4]oxazin-6-ylmethoxy]-5-(toluene-4-sulfonyloxymethyl)piperidine-1-carboxylate

The title compound is obtained as a yellow oil starting from benzyl (3S,4R,5R)-3-hydroxy-methyl-4-(4-methoxyphenyl)-5-[4-(3-methoxypropyl)-3,4-dihydro-2H-benzo[1,4]oxazin-6-ylmethoxy]piperidine-1-carboxylate in analogy to method H. The crude title compound is used in the next stage. Rf=0.39 (EtOAc-heptane=2:1).

### c) Benzyl (3S,4R,5R)-3-hydroxymethyl-4-(4-methoxyphenyl)-5-[4-(3-methoxypropyl)-3,4-dihydro-2H-benzo[l 4]oxazin-6-ylmethoxy]piperidine-1-carboxylate

The title compound is obtained as a yellow oil starting from benzyl (3S,4R,5R)-3-hydroxy-methyl-4-(4-methoxyphenyl)-5-[4-(3-methoxypropyl)-3-oxo-3,4-dihydro-2H-benzo[1,4]oxazin-6-ylmethoxy]piperidine-1-carboxylate in analogy to the process described in Example 11d. Rf=0.18 (EtOAc-heptane=2:1); Rt=4.79 (Gradient I).

### d) Benzyl (3S,4R,5R)-3-hydroxymethyl-4-(4-methoxyphenyl)-5-[4-(3-methoxypropyl)-3-oxo-3,4-dihydro-2H-benzo[1,4]oxazin-6-ylmethoxy]piperidine-1-carboxylate

A solution of 1.87 g of benzyl (3R,4R,5S)-4-(4-methoxyphenyl)-3-[4-(3-methoxypropyl)-3-oxo-3,4-dihydro-2H-benzo[1,4]oxazin-6-ylmethoxy]-5-trityloxymethylpiperidine-1-carboxylate in 20 ml of methanol and 4 ml of tetrahydrofuran is mixed with 1.53 g of P-toluenesulfonic acid monohydrate and stirred at room temperature for 1.5 hours. The reaction mixture is poured into saturated aqueous sodium bicarbonate solution and extracted with dichloro-methane (3×). The combined organic phases are dried with sodium sulfate and evaporated. The crude title compound is used in the next stage.

### e) Benzyl (3R,4R,5S)-4-(4-methoxyphenyl)-3-[4-(3-methoxypropyl)-3-oxo-3,4-dihydro-2H-benzo[1,4]oxazin-6-ylmethoxy]-5-trityloxymethylpiperidine-1-carboxylate

The title compound is obtained as a colourless wax starting from benzyl (3R,4R,5S)-3-hydroxy-4-(4-methoxyphenyl)-5-trityloxymethylpiperidine-1-carboxylate in analogy to method D. Rf=0.26 (EtOAc-heptane=1:1); Rt=6.25 (Gradient I).

### f) Benzyl (3R,4R,5S)-3-hydroxy-4-(4-methoxyphenyl)-5-trityloxymethylpiperidine-1-carboxylate

The title compound is obtained as a yellow oil starting from (3R,4R,5S)-1-benzyl-4-(4-methoxyphenyl)-5-trityloxymethylpiperidin-3-ol (L)-(+)-mandelate [303043-54-1] in analogy to the process described in method B (3:1 methanol-tetrahydrofuran is used as solvent) and in Example 11h. The crude title compound is used in the next stage. Rf=0.25 (EtOAc-heptane=1:1).

The following compounds are prepared in an analogous manner to the process described in Example 87:

### Examples

### 88 tert-Butyl-{(3S,4R,5R)-4-(4-methoxyphenyl)-5-[4-(3-methoxypropyl)-3,4-dihydro-2H-benzo[1,4]oxazin-6-ylmethoxy]piperidin-3-ylmethyl}amine

### 89 (2-Methoxyethyl)-{(3R,4R,5R)-4-(4-methoxyphenyl)-5-[4-(3-methoxypropyl)-3,4-dihydro-2H-benzo[1,4]oxazin-6-ylmethoxy]piperidin-3-ylmethyl}amine

### 90 6-[(3R,4R,5S)-4-(4-Methoxyphenyl)-5-morpholin-4-ylmethylpiperidin-3-yloxymethyl]-4-(3-methoxypropyl)-3,4-dihydro-2H-benzo[1,4]oxazine

### Example 91

### N-{(3S,4R,5R)-4-(4-Methoxyphenyl)-5-[4-(3-methoxypropyl)-3,4-dihydro-2H-benzo[1,4]oxazin-6-ylmethoxy]piperidin-3-ylmethyl}acetamide

The title compound is prepared from benyzl (3R,4R,5R)-3-(acetylaminomethyl)-4-(4-methoxyphenyl)-5-[4-(3-methoxypropyl)-3,4-dihydro-2H-benzo[1,4]oxazin-6-ylmethoxy]piperidine-1-carboxylate in analogy to method B.

The starting materials are prepared as follows:

### a) Benzyl (3R,4R,5R)-3-(acetylaminomethyl)-4-(4-methoxyphenyl)-5-[4-(3-methoxypropyl)-3,4-dihydro-2H-benzo[1,4]oxazin-6-ylmethoxy]piperidine-1-carboxylate

3 mmol of triethylamine are added to a solution of 1 mmol of benzyl (3R,4R,5R)-3-aminomethyl-4-(4-methoxyphenyl)-5-[4-(3-methoxypropyl)-3,4-dihydro-2H-benzo[1,4]oxazin-6-ylmethoxy]piperidine-1-carboxylate and 1.1 mmol of acetyl chloride in 20 ml of dichloro-methane at 0° C. After 1.5 hours, the reaction mixture is poured into 1 M sodium bicarbonate solution and extracted with tert-butyl methyl ether (3×), and the combined organic phases are washed with brine, dried with sodium sulfate and evaporated. The title compound is obtained as a yellowish oil from the residue by flash chromatography (SiO₂ 60F). Rf=0.29 (dichloromethane-methanol-25% conc. ammonia=200:20:1); Rt=4.58 (Gradient I).

### b) Benzyl (3R,4R,5R)-3-aminomethyl-4-(4-methoxyphenyl)-5-[4-(3-methoxypropyl)-3,4-dihydro-2H-benzo[1,4]oxazin-6-ylmethoxy]piperidine-1-carboxylate

A solution of 0.195 ml of 25% conc. ammonia in 0.9 ml of methanol is added to a solution of 200 mg of benzyl (3S,4R,5R)-3-azidomethyl-4-(4-methoxyphenyl)-5-[4-(3-methoxypropyl)-3,4-dihydro-2H-benzo[1,4]oxazin-6-ylmethoxy]piperidine-1-carboxylate in 0.95 ml of tetrahydrofuran and 0.23 ml of water at room temperature. After addition of 128 mg of triphenylphosphine, the reaction mixture is stirred at room temperature for 16 hours. The mixture is diluted with ethyl acetate and washed with half-saturated aqueous sodium bicarbonate solution (2×), dried with sodium sulfate and evaporated. The title compound is obtained as a colourless oil from the residue by flash chromatography (SiO₂ 60F). Rf=0.31 (dichloromethane-methanol-25% conc. ammonia=200:20:1); Rt=4.24 (Gradient I).

### c) Benzyl (3S,4R,5R)-3-azidomethyl-4-(4-methoxyphenyl)-5-[4-(3-methoxypropyl)-3,4-dihydro-2H-benzo[1,4]oxazin-6-ylmethoxy]piperidine-1-carboxylate

A solution of 0.50 mmol of benzyl (3R,4R,5S)-4-(4-methoxyphenyl)-3-[4-(3-methoxypropyl)-3,4-dihydro-2H-benzo[1,4]oxazin-6-ylmethoxy]-5-(toluene-4-sulfonyloxymethyl)piperidine-1-carboxylate (Example 87b) and 2 mmol of sodium azide in 5 ml of DMPU is stirred at 80° C. for 4 hours. The reaction mixture is diluted with water and extracted with tert-butyl methyl ether (3×). The combined organic phases are dried with sodium sulfate and evaporated. The title compound is obtained as a yellowish oil from the residue by flash chromatography (SiO₂ 60F). Rf=0.33 (EtOAc-heptane=1:1); Rt=5.61 (Gradient I).

The following compounds are prepared in an analogous manner to the process described in Example 91.

### Examples

### 92 N-{(3S,4R,5R)-4-(4-Methoxyphenyl)-5-[4-(3-methoxypropyl)-3,4-dihydro-2H-benzo[1,4]oxazin-6-ylmethoxy]piperidin-3-ylmethyl}propionamide

### 115 Morpholine-4-carboxylic acid {(3S,4R,5R)-4-(4-methoxyphenyl)-5-[4-(3-methoxy-propyl)-3,4-dihydro-2H-benzo[1,4]oxazin-6-ylmethoxy]piperidin-3-ylmethyl}amide

using morpholine-4-carbonyl chloride [15159-40-7]

### 116 Pentanoic acid {(3S,4R,5R)-4-(4-methoxyphenyl)-5-[4-(3-methoxypropyl)-3,4-dihydro-2H-benzo[1,4]oxazin-6-ylmethoxy]piperidin-3-ylmethyl}amide

### 123 1-{(3S,4R,5R)-4-(4-Methoxyphenyl)-5-[4-(3-methoxypropyl)-3,4-dihydro-2H-benzo[1,4]oxazin-6-ylmethoxy]piperidin-3-ylmethyl}-3-propylurea

using propylcarbamoyl chloride [41891-16-1]

### 124 1-Cyclopentyl-3-{(3S,4R,5R)-4-(4-methoxyphenyl)-5-[4-(3-methoxypropyl)-3,4-dihydro-2H-benzo[1,4]oxazin-6-ylmethoxy]piperidin-3-ylmethyl}urea

using cyclopentylcarbamoyl chloride [80413-82-7]

### 127 Cyclopentanecarboxylic acid {(3S,4R,5R)-4-(4-methoxy-phenyl)-5-[4-(3-methoxy-propyl)-3,4-dihydro-2H-benzo[1,4]oxazin-6-ylmethoxy]-piperidin-3-ylmethyl}-amide

using cyclopentanecarbonyl chlorid [4524-93-0]

### 128 N-{(3S,4R,5R)-4-(4-Methoxy-phenyl)-5-[4-(3-methoxy-propyl)-3,4-dihydro-2H-benzo[1,4]oxazin-6-ylmethoxy]-piperidin-3-ylmethyl}-2,2-dimethyl-propionamide

using pivaloyl chlorid [3282-30-2]

### 129 {(3S,4R,5R)-4-(4-Methoxy-phenyl)-5-[4-(3-methoxy-propyl)-3,4-dihydro-2H-benzo[1,4]oxazin-6-ylmethoxy]-piperidin-3-ylmethyl}-carbamic acid methyl ester

using methyl chloroformate [79-22-1]

### 133 1-{(3S,4R,5R)-4-(4-Methoxy-phenyl)-5-[4-(3-methoxy-propyl)-3,4-dihydro-2H-benzo[1,4]oxazin-6-ylmethoxy]-piperidin-3-ylmethyl}-3-methyl-urea

using N-succinimidyl N-methylcarbamate [18342-66-0]

### 134 3-{(3S,4R,5R)-4-(4-Methoxy-phenyl)-5-[4-(3-methoxy-propyl)-3,4-dihydro-2H-benzo[1,4]oxazin-6-ylmethoxy]-piperidin-3-ylmethyl}-1,1-dimethyl-urea

using N,N-dimethylcarbamoyl chloride [79-44-7]

### 130 N-{(3S,4R,5R)-4-(4-Methoxy-phenyl)-5-[4-(3-methoxy-propyl)-3,4-dihydro-2H-benzo[1,4]oxazin-6-ylmethoxy]-piperidin-3-ylmethyl}-butyramide

using butyryl chloride [1,4]-75-3]

### 131 N—((R)-2-{(3S,4R,5R)-4-(4-Methoxy-phenyl)-5-[4-(3-methoxy-propyl)-3,4-dihydro-2H-benzo[1,4]oxazin-6-ylmethoxy]-piperidin-3-yloxy}-1-methyl-ethyl)-isobutyramide

starting from (3S,4R,5R)-3-((S)-2-methanesulfonyloxy-propoxy)-4-(4-methoxy-phenyl)-5-[4-(3-methoxy-propyl)-3,4-dihydro-2H-benzo[1,4]oxazin-6-ylmethoxy]-piperidine-1-carboxylic acid benzyl ester using isobutyryl chloride [79-30-1]

The starting materials are prepared as follows:

### a) (3S,4R,5R)-3-((S)-2-Methanesulfonyloxy-propoxy)-4-(4-methoxy-phenyl)-5-[4-(3-methoxy-propyl)-3,4-dihydro-2H-benzo[1,4]oxazin-6-ylmethoxy]-piperidine-1-carboxylic Acid Benzyl Ester

The title compound is obtained as a brown oil from (3S,4R,5R)-3-((S)-2-hydroxy-propoxy)-4-(4-methoxy-phenyl)-5-[4-(3-methoxy-propyl)-3,4-dihydro-2H-benzo[1,4]oxazin-6-ylmethoxy]-piperidine-1-carboxylic acid benzyl ester in analogy to the process used in Example 64b. Rt=5.24 (Gradient I).

### b) (3S,4R,5R)-3-((S)-2-Hydroxy-propoxy)-4-(4-methoxy-phenyl)-5-[4-(3-methoxy-propyl)-3,4-dihydro-2H-benzo[1,4]oxazin-6-ylmethoxy]-piperidine-1-carboxylic Acid Benzyl Ester

A solution of 6.78 mmol (3R,4R,5S)-4-(4-methoxy-phenyl)-3-[4-(3-methoxy-propyl)-3,4-dihydro-2H-benzo[1,4]oxazin-6-ylmethoxy]-5-((S)-1-oxiranylmethoxy)-piperidine-1-carboxylic acid benzyl ester in 70 ml ethanol is treated with 20.36 mmol sodium borohydrid. The solution is stirred at 45° C. over night, cooled to room temperature, diluted with tert-butyl methyl ether and washed successively with saturated ammonium chloride solution, water and brine. The aqueous phases are extracted (3×) with dichloromethane. The combined organic phases are dried with sodium sulfate and evaporated. The title compound is obtained as a yellow oil from the residue by means of flash chromatography (SiO₂ 60F). Rf=0.20 (EtOAc-heptane 2:1); Rt=4.96 (Gradient I).

### c) (3R,4R,5S)-4-(4-Methoxy-phenyl)-3-[4-(3-methoxy-propyl)-3,4-dihydro-2H-benzo[1,4]oxazin-6-ylmethoxy]-5-((S)-1-oxiranylmethoxy)-piperidine-1-carboxylic Acid Benzyl Ester

The title compound is obtained as a yellow oil from (3S,4S,5R)-3-hydroxy-4-(4-methoxy-phenyl)-5-[4-(3-methoxy-propyl)-3,4-dihydro-2H-benzo[1,4]oxazin-6-ylmethoxy]-piperidine-1-carboxylic acid benzyl ester (Example 19c) in analogy to the process used in Example 36a, using toluol-4-sulfonsäure (S)-1-oxiranymethyl ester [70987-78-9]. Rf=0.18 (EtOAc-heptane 1:1); Rt=5.12 (Gradient I).

### 132 N—((R)-2-{(3S,4R,5R)-4-(4-Methoxy-phenyl)-5-[4-(3-methoxy-propyl)-3,4-dihydro-2H-benzo[1,4]oxazin-6-ylmethoxy]-piperidin-3-yloxy}-1-methyl-ethyl)-propionamide

starting from (3S,4R,5R)-3-((S)-2-methanesulfonyloxy-propoxy)-4-(4-methoxy-phenyl)-5-[4-(3-methoxy-propyl)-3,4-dihydro-2H-benzo[1,4]oxazin-6-ylmethoxy]-piperidine-1-carboxylic acid benzyl ester (Example 131a) using propanoyl chloride [79-03-8]

### 135 N-Ethyl-N—((R)-2-{(3S,4R,5R)-4-(4-methoxy-phenyl)-5-[4-(3-methoxy-propyl)-3,4-dihydro-2H-benzo[1,4]oxazin-6-ylmethoxy]-piperidin-3-yloxy}-1-methyl-ethyl)-acetamide

starting from (3S,4R,5R)-3-((R)-2-ethylamino-propoxy)-4-(4-methoxy-phenyl)-5-[4-(3-methoxy-propyl)-3,4-dihydro-2H-benzo[1,4]oxazin-6-ylmethoxy]-piperidine-1-carboxylic acid benzyl ester

The starting materials are prepared as follows:

### a) (3S,4R,5R)-3-((R)-2-Ethylamino-propoxy)-4-(4-methoxy-phenyl)-5-[4-(3-methoxy-propyl)-3,4-dihydro-2H-benzo[1,4]oxazin-6-ylmethoxy]-piperidine-1-carboxylic Acid Benzyl Ester

A solution of 1.82 mmol (3S,4R,5R)-3-((S)-2-methanesulfonyloxy-propoxy)-4-(4-methoxy-phenyl)-5-[4-(3-methoxy-propyl)-3,4-dihydro-2H-benzo[1,4]oxazin-6-ylmethoxy]-piperidine-1-carboxylic acid benzyl ester (Example 131a) in 25 ml of ethanol is treated with 100.32 mmol of ethylamine. The solution is stirred at 50° C. for 4 days, cooled to room temperature, evaporated and dissolved in tert-butyl methyl ether. The organic phase is washed successively with water and brine. The aqueous phases are extracted (3×) with tert-butyl methyl ether. The combined organic phases are dried with sodium sulfate and evaporated. The title compound is obtained as a yellow oil from the residue by means of flash chromatography (SiO₂ 60F). Rf=0.16 (EtOAc-triethylamine 100:1); Rt=4.61 (Gradient I).

### 136 N—((R)-2-{(3S,4R,5R)-4-(4-Methoxy-phenyl)-5-[4-(3-methoxy-propyl)-3,4-dihydro-2H-benzo[1,4]oxazin-6-ylmethoxy]-piperidin-3-yloxy}-1-methyl-ethyl)-N-propyl-acetamide

starting from (3R,4R,5S)-4-(4-methoxy-phenyl)-3-[4-(3-methoxy-propyl)-3,4-dihydro-2H-benzo[1,4]oxazin-6-ylmethoxy]-5-((R)-2-propylamino-propoxy)-piperidine-1-carboxylic acid benzyl ester.

The starting materials are prepared as follows:

### a) (3R,4R,5S)-4-(4-Methoxy-phenyl)-3-[4-(3-methoxy-propyl)-3,4-dihydro-2H-benzo[1,4]oxazin-6-ylmethoxy]-5-((R)-2-propylamino-propoxy)-piperidine-1-carboxylic Acid Benzyl Ester

The title compound is obtained as a yellow oil from (3S,4R,5R)-3-((S)-2-methanesulfonyloxy-propoxy)-4-(4-methoxy-phenyl)-5-[4-(3-methoxy-propyl)-3,4-dihydro-2H-benzo[1,4]oxazin-6-ylmethoxy]-piperidine-1-carboxylic acid benzyl ester (Example 131a) in analogy to the process used in Example 135a, using propylamine. Rf=0.19 (EtOAc-triethylamine 100:1); Rt=4.75 (Gradient I).

### Example 93

### (R)-1-{(3S,4R,5R)-4-[4-((R)-4-Methoxy-3-methylbutylsulfanyl)phenyl]-5-[4-(3-methoxy-propyl)-3,4-dihydro-2H-benzo[1,4]oxazin-6-ylmethoxy]piperidin-3-yloxy}propan-2-ol

A solution of 0.4 mmol of methyl (3R,4R,5S)-4-[4-((R)-4-methoxy-3-methylbutylsulfanyl)-phenyl]-3-[4-(3-methoxypropyl)-3,4-dihydro-2H-benzo[1,4]oxazin-6-ylmethoxy]-5-((R)-1-oxiranylmethoxy)piperidine-1-carboxylate in 4 ml of ethanol and 1 ml of tetrahydrofuran is mixed with 12 mmol of sodium borohydride. The reaction solution is stirred at 50° C. for 15 hours and then mixed with 3 ml of dioxane, 3.6 ml of aqueous 40% potassium hydroxide solution and 1 ml of methanol. The reaction mixture is stirred at 90° C. for 1 hour and then, at room temperature, poured into water and diluted with tert-butyl methyl ether. The aqueous phase is extracted with tert-butyl methyl ether (2×). The combined organic phases are washed with brine, dried with sodium sulfate and evaporated. The title compound is obtained from the residue by flash chromatography (SiO₂ 60F).

The starting material is prepared as follows:

### a) Methyl (3R,4R,5S)-4-[4-((R)-4-methoxy-3-methylbutylsulfanyl)phenyl]-3-[4-(3-methoxy-propyl)-3,4-dihydro-2H-benzo[1,4]oxazin-6-ylmethoxy]-5-((R)-1-oxiranylmethoxy)piperidine-1-carboxylate

0.5 mmol of methyl (3S,4S,5R)-3-hydroxy-4-[4-((R)-4-methoxy-3-methylbutylsulfanyl)-phenyl]-5-[4-(3-methoxypropyl)-3,4-dihydro-2H-benzo[1,4]oxazin-6-ylmethoxy]piperidine-1-carboxylate (Example 83a) and 1 mmol of (R)-1-oxiranylmethyl toluene-4-sulfonate [113826-06-5] are reacted in analogy to Example 36a. The title compound is identified on the basis of the Rf.

The following compounds are prepared in an analogous manner to the process described in Example 93.

### Examples

### 94 (R)-1-{(3S,4R,5R)-5-[4-(3-Methoxypropyl)-3,4-dihydro-2H-benzo[1,4]oxazin-6-ylmethoxy]-4-[4-(3-methoxypropylsulfanyl)phenyl]piperidin-3-yloxy}propan-2-ol

### 95 (R)-1-{(3S,4R,5R)-4-[4-(4-Methoxybutylsulfanyl)phenyl]-5-[4-(3-methoxypropyl)-3,4-dihydro-2H-benzo[1,4]oxazin-6-ylmethoxy]piperidin-3-yloxy}propan-2-ol

### 102 (R)-1-{(3S,4R,5R)-5-[4-(3-Methoxypropyl)-3,4-dihydro-2H-benzo[1,4]oxazin-6-ylmethoxy]-4-[4-(3-methylsulfanylpropoxy)phenyl]piperidin-3-yloxy}propan-2-ol

starting from benzyl (3S,4S,5R)-3-hydroxy-5-[4-(3-methoxypropyl)-3,4-dihydro-2H-benzo[1,4]oxazin-6-ylmethoxy]-4-[4-(3-methylsulfanylpropoxy)phenyl]piperidine-1-carboxylate (Example 80a).

### Example 96

### (S)-4-{(3S,4S,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}butan-2-ol

The title compound is prepared from 0.63 g of benzyl (3S,4S,5R)-3-((S)-3-hydroxybutoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate in analogy to method B.

The starting materials are prepared as follows:

### a) Benzyl (3S,4S,5R)-3-((S)-3-hydroxybutoxy)-4-[4-(3-methoxypropoxy)phenyl]-5-[4-(3-methoxypropyl)-3,4-dihydro-2H-benzo[1,4]oxazin-6-ylmethoxy]piperidine-1-carboxylate

The title compound is obtained as a yellow oil from 0.807 g of benzyl (3S,4S,5R)-3-[(S)-3-(tert-butyldimethylsilanyloxy)butoxy]-4-[4-(3-methoxypropoxy)phenyl]-5-[4-(3-methoxy-propyl)-3,4-dihydro-2H-benzo[1,4]oxazin-6-ylmethoxy]piperidine-1-carboxylate in analogy to method J. Rf=0.12 (EtOAc-heptane 2:1); Rt=5.08 (Gradient I).

### b) Benzyl (3S,4S,5R)-3-[(S)-3-(tert-butyldimethylsilanyloxy)butoxy]-4-[4-(3-methoxy-propoxy)phenyl]-5-[4-(3-methoxypropyl)-3,4-dihydro-2H-benzo[1,4]oxazin-6-ylmethoxy]piperidine-1-carboxylate

0.15 g of sodium hydride (60% dispersion in oil) is added to a solution of 1.68 g of benzyl (3S,4S,5R)-3-hydroxy-4-[4-(3-methoxypropoxy)phenyl]-5-[4-(3-methoxypropyl)-3,4-dihydro-2H-benzo[1,4]oxazin-6-ylmethoxy]piperidine-1-carboxylate (Example 20b) in 10 ml of DMF at 0° C., and the mixture is stirred for 1 hour. It is then cooled to −5° C. and 1.25 g of tert-butyl ((S)-3-iodo-1-methylpropoxy)dimethylsilane [134510-70-6] are added dropwise over the course of 1 hour. The reaction mixture is stirred at −5° C. for 3 hours and then warmed to room temperature. It is then diluted with tert-butyl methyl ether and poured into ice-water. The resulting mixture is extracted three times with tert-butyl methyl ether. The combined organic phases are washed with brine, dried with sodium sulfate and evaporated. The title compound is obtained as a yellow oil from the residue by flash chromatography (SiO₂ 60F). Rf=0.45 (EtOAc-heptane 2:1).

The following compounds are prepared in an analogous manner to the process described in Example 96:

### Examples

### 97 (R)-4-{(3S,4S,5R)-4-[4-(3-Methoxypropoxy)phenyl]-5-[4-(3-methoxypropyl)-3,4-dihydro-2H-benzo[1,4]oxazin-6-ylmethoxy]piperidin-3-yloxy}butan-2-ol

### 107 (S)-4-{(3S,4S,5R)-4-[4-(4-Methoxybutoxy)phenyl]-5-[4-(3-methoxypropyl)-3,4-dihydro-2H-benzo[1,4]oxazin-6-ylmethoxy]piperidin-3-yloxy}butan-2-ol

starting from benzyl (3S,4S,5R)-3-hydroxy-4-[4-(4-methoxybutoxy)phenyl]-5-[4-(3-methoxy-propyl)-3,4-dihydro-2H-benzo[1,4]oxazin-6-ylmethoxy]piperidine-1-carboxylate (Example 78a)

### 109 (R)-4-{(3S,4S,5R)-4-[4-(4-Methoxybutoxy)phenyl]-5-[4-(3-methoxypropyl)-3,4-dihydro-2H-benzo[1,4]oxazin-6-ylmethoxy]piperidin-3-yloxy}butan-2-ol

starting from benzyl (3S,4S,5R)-3-hydroxy-4-[4-(4-methoxybutoxy)phenyl]-5-[4-(3-methoxy-propyl)-3,4-dihydro-2H-benzo[1,4]oxazin-6-ylmethoxy]piperidine-1-carboxylate (Example 78a)

### Example 98

### (R)-1-{(3S,4R,5R)-4-[4-(4-Methoxybutoxy)phenyl]-5-[4-(3-methoxypropyl)-3,4-dihydro-2H-benzo[1,4]oxazin-6-ylmethoxy]piperidin-3-yloxy}propan-2-ol

The title compound is prepared from 0.282 g of benzyl (3R,4R,5S)-4-[4-(4-methoxybutoxy)-phenyl]-3-[4-(3-methoxypropyl)-3,4-dihydro-2H-benzo[1,4]oxazin-6-ylmethoxy]-5-((R)-1-oxiranylmethoxy)piperidine-1-carboxylate in analogy to method B.

The starting material is prepared as follows:

### a) Benzyl (3R,4R,5S)-4-[4-(4-methoxybutoxy)phenyl]-3-[4-(3-methoxypropyl)-3,4-dihydro-2H-benzo[1,4]oxazin-6-ylmethoxy]-5-((R)-1-oxiranylmethoxy)piperidine-1-carboxylate

0.32 g of benzyl (3S,4S,5R)-3-hydroxy-4-[4-(4-methoxybutoxy)phenyl]-5-[4-(3-methoxy-propyl)-3,4-dihydro-2H-benzo[1,4]oxazin-6-ylmethoxy]piperidine-1-carboxylate (Example 78a) and 0.227 g of (R)-1-oxiranylmethyl toluene-4-sulfonate [113826-06-5] are reacted in analogy to Example 36a. The title compound is obtained as a yellowish oil. Rf=0.35 (EtOAc-heptan 2:1); Rt=5.36 (Gradient I).

The following compound is prepared in an analogous manner to the process described in Example 98:

### Example 105

### (S)-1-{(3S,4R,5R)-4-[4-(4-Methoxybutoxy)phenyl]-5-[4-(3-methoxypropyl)-3,4-dihydro-2H-benzo[1,4]oxazin-6-ylmethoxy]piperidin-3-yloxy}propan-2-ol

using (S)-1-oxiranylmethyl toluene-4-sulfonate [70987-78-9]

### Example 117

### Tetrahydropyran-4-carboxylic acid {(3S,4R,5R)-4-(4-methoxyphenyl)-5-[4-(3-methoxy-propyl)-3,4-dihydro-2H-benzo[1,4]oxazin-6-ylmethoxy]piperidin-3-ylmethyl}amide

The title compound is prepared from benzyl (3R,4R,5R)-4-(4-methoxyphenyl)-3-[4-(3-methoxypropyl)-3,4-dihydro-2H-benzo[1,4]oxazin-6-ylmethoxy]-5-{[(tetrahydropyran-4-carbonyl)amino]methyl}piperidine-1-carboxylate in analogy to method B.

The starting materials are prepared as follows:

### a) Benzyl (3R,4R,5R)-4-(4-methoxyphenyl)-3-[4-(3-methoxypropyl)-3,4-dihydro-2H-benzo[1,4]oxazin-6-ylmethoxy]-5-{[(tetrahydropyran-4-carbonyl)amino]-methyl}piperidine-1-carboxylate

5 mmol of triethylamine and 1 mmol of propanephosphonic anhydride [68957-94-8, T3P] (50% in ethyl acetate) are successively added to a solution of 1 mmol of benzyl (3R,4R,5R)-3-aminomethyl-4-(4-methoxyphenyl)-5-[4-(3-methoxypropyl)-3,4-dihydro-2H-benzo[1,4]oxazin-6-ylmethoxy]piperidine-1-carboxylate (Example 91b) and 1.1 mmol of tetrahydropyran-4-carboxylic acid [5337-03-1] in 20 ml of dichloromethane at room temperature. After 12 hours, the reaction mixture is diluted with dichloromethane and washed successively with 1 N HCl and brine, dried with sodium sulfate and evaporated. The title compound is obtained as a colourless oil from the residue by flash chromatography (SiO₂ 60F). Rf=0.13 (EtOAc); Rt=4.63 (Gradient I).

The following compounds are prepared in an analogous manner to the process described in Example 117:

### Examples

### 118 2-Cyclopentyl-N-{(3S,4R,5R)-4-(4-methoxyphenyl)-5-[4-(3-methoxypropyl)-3,4-dihydro-2H-benzo[1,4]oxazin-6-ylmethoxy]piperidin-3-ylmethyl}acetamide

using cyclopentylacetic acid [1123-00-8]

### 119 (meso-1S,5R,6R)-3-Oxabicyclo[3.1.0]hexan-6-carboxylic Acid {(3S,4R,5R)-4-(4-methoxyphenyl)-5-[4-(3-methoxypropyl)-3,4-dihydro-2H-benzo[1,4]oxazin-6-ylmethoxy]piperidin-3-ylmethyl}amide

using (meso-1S,5R,6R)-3-oxabicyclo[3.1.0]hexane-6-carboxylic acid [55780-88-6]

### 120 N-{(3S,4R,5R)-4-(4-Methoxyphenyl)-5-[4-(3-methoxypropyl)-3,4-dihydro-2H-benzo[1,4]oxazin-6-ylmethoxy]piperidin-3-ylmethyl}-2-(meso-1R,5S,6S)-3-oxa-bicyclo[3.1.0]hex-6-ylacetamide

using (meso-1R,5S,6S)-(3-oxabicyclo[3.1.0]hex-6-yl)acetic acid

The starting materials are prepared as follows:

### a) (meso-1R,5S,6S)-(3-Oxabicyclo[3.1.0]hex-6-yl)acetic Acid

3 mmol of triethylamine and 0.5 mmol of silver trifluoroacetate are added to a solution of 1 mmol of 1-diazo-3-(meso-1R,5S,6S)-3-oxabicyclo[3.1.0]hex-6-ylpropan-2-one in 70 ml 10:1 tetrahydrofuran-water at −15° C. The reaction mixture is warmed to room temperature and stirred at room temperature for 2 hours. It is diluted with tert-butyl methyl ether, washed with 1 M HCl and brine, dried with sodium sulfate and evaporated. The title compound is identified from the residue on the basis of the Rf by flash chromatography (SiO₂ 60F).

### b) 1-Diazo-3-(meso-1R,5S,6S)-3-oxabicyclo[3.1.0]hex-6-yl-propan-2-one

1.2 mmol of triethylamine and 1 mmol of ethyl chloroformate are added to a solution of 1 mmol of (meso-1S,5R,6R)-3-oxabicyclo[3.1.0]hexane-6-carboxylic acid [55780-88-6] in 60 ml of tetrahydrofuran at −15° C. The reaction mixture is warmed to −5° C. and stirred at this temperature for 1 hour. It is cooled to −30° C., and 2.5 mmol of a diazomethane solution in ether are added, and the mixture is stirred overnight. It is diluted with tert-butyl methyl ether, washed with saturated aqueous sodium bicarbonate solution and brine, dried with sodium sulfate and evaporated. The title compound is identified from the residue on the basis of the Rf by flash chromatography (SiO₂ 60F).

### 121 4-Methoxycyclohexanecarboxylic Acid {(3S,4R,5R)-4-(4-methoxyphenyl)-5-[4-(3-methoxypropyl)-3,4-dihydro-2H-benzo[1,4]oxazin-6-ylmethoxy]-piperidin-3-ylmethyl}-amide

using 4-methoxycyclohexanecarboxylic acid [99183-14-9]

### 122 N-{(3S,4R,5R)-4-(4-Methoxyphenyl)-5-[4-(3-methoxypropyl)-3,4-dihydro-2H-benzo[1,4]oxazin-6-ylmethoxy]piperidin-3-ylmethyl}-2-(tetrahydropyran-4-yl)acetamide

using (tetrahydropyran-4-yl)acetic acid [85064-61-5]

### Example 138

### N-{(3S,4R,5R)-4-[4-(4-Methoxy-butoxy)-phenyl]-5-[4-(3-methoxy-propyl)-3,4-dihydro-2H-benzo[1,4]oxazin-6-ylmethoxy]-piperidin-3-ylmethyl}-acetamide

The title compound is obtained from N-[(3R,4R,5R)-4-[4-(4-methoxy-butoxy)-phenyl]-5-[4-(3-methoxy-propyl)-3,4-dihydro-2H-benzo[1,4]oxazin-6-ylmethoxy]-1-(toluene-4-sulfonyl)-piperidin-3-ylmethyl]-acetamide in analogy to method L.

The starting materials are prepared as follows:

### a) N-[(3R,4R,5R)-4-[4-(4-Methoxy-butoxy)-phenyl]-5-[4-(3-methoxy-propyl)-3,4-dihydro-2H-benzo[1,4]oxazin-6-ylmethoxy]-1-(toluene-4-sulfonyl)-piperidin-3-ylmethyl]-acetamide

The title compound is obtained as a white foam from toluene-4-sulfonic acid (3S,4R,5R)-4-[4-(4-methoxy-butoxy)-phenyl]-5-[4-(3-methoxy-propyl)-3,4-dihydro-2H-benzo[1,4]oxazin-6-ylmethoxy]-1-(toluene-4-sulfonyl)-piperidin-3-ylmethyl ester in analogy to the process described in Example 91a, b and c. Rf=0.72 (Dichloromethane-methanol-25% conc. ammonia=200:20:1); Rt=4.86 (Gradient I).

### b) Toluene-4-sulfonic acid (3S,4R,5R)-4-[4-(4-methoxy-butoxy)-phenyl]-5-[4-(3-methoxy-propyl)-3,4-dihydro-2H-benzo[1,4]oxazin-6-ylmethoxy]-1-(toluene-4-sulfonyl)-piperidin-3-ylmethyl Ester

The title compound is obtained as a yellowish oil from [(3S,4R,5R)-4-[4-(4-methoxy-butoxy)-phenyl]-5-[4-(3-methoxy-propyl)-3,4-dihydro-2H-benzo[1,4]oxazin-6-ylmethoxy]-1-(toluene-4-sulfonyl)-piperidin-3-yl]-methanol in analogy to method H. Rf=0.46 (EtOAc-heptane=2:1); Rt=5.76 (Gradient I).

### c) [(3S,4R,5R)-4-[4-(4-Methoxy-butoxy)-phenyl]-5-[4-(3-methoxy-propyl)-3,4-dihydro-2H-benzo[1,4]oxazin-6-ylmethoxy]-1-(toluene-4-sulfonyl)-piperidin-3-yl]-methanol

The title compound is obtained as a yellow oil from 4-[(3S,4R,5R)-3-hydroxymethyl-5-[4-(3-methoxy-propyl)-3,4-dihydro-2H-benzo[1,4]oxazin-6-ylmethoxy]-1-(toluene-4-sulfonyl)-piperidin-4-yl]-phenol and 1-bromo-3-methoxy-propane [4457-67-4] in analogy to method F. Rf=0.28 (EtOAc-heptane=2:1); Rt=5.07 (Gradient I).

### d) 4-[(3S,4R,5R)-3-Hydroxymethyl-5-[4-(3-methoxy-propyl)-3,4-dihydro-2H-benzo[1,4]oxazin-6-ylmethoxy]-1-(toluene-4-sulfonyl)-piperidin-4-yl]-phenol

A solution of 1 mmol of [(3S,4R,5R)-4-(4-methoxy-phenyl)-5-[4-(3-methoxy-propyl)-3,4-dihydro-2H-benzo[1,4]oxazin-6-ylmethoxy]-1-(toluene-4-sulfonyl)-piperidin-3-yl]-methanol in 5 ml of N,N-dimethylformamide is mixed with 5 mmol of sodium ethanethiolate, and the resulting suspension is heated at 130° C. overnight. It is diluted with 1N HCl and extracted with ethyl acetate (2×). The combined organic phases are dried with sodium sulfate and evaporated. The title compound is obtained as a yellow foam from the residue by flash chromatography (SiO₂ 60F). Rf=0.13 (EtOAc-heptane=2:1); Rt=4.35 (Gradient I).

### e) [(3S,4R,5R)-4-(4-Methoxy-phenyl)-5-[4-(3-methoxy-propyl)-3,4-dihydro-2H-benzo[1,4]oxazin-6-ylmethoxy]-1-(toluene-4-sulfonyl)-piperidin-3-yl]-methanol

The title compound is obtained as a colourless oil from {(3S,4R,5R)-4-(4-methoxy-phenyl)-5-[4-(3-methoxy-propyl)-3,4-dihydro-2H-benzo[1,4]oxazin-6-ylmethoxy]-piperidin-3-yl}-methanol in analogy to the process described in Example if. Rf=0.25 (EtOAc-heptane=2:1); Rt=4.83 (Gradient I).

### f) {(3S,4R,5R)-4-(4-Methoxy-phenyl)-5-[4-(3-methoxy-propyl)-3,4-dihydro-2H-benzo[1,4]oxazin-6-ylmethoxy]-piperidin-3-yl}-methanol

The title compound is obtained as a yellow oil from (3S,4R,5R)-3-hydroxymethyl-4-(4-methoxy-phenyl)-5-[4-(3-methoxy-propyl)-3,4-dihydro-2H-benzo[1,4]oxazin-6-ylmethoxy]-piperidin-1-carboxylic acid benzyl ester (Example 87c) in analogy to method B. Rt=3.34 (Gradient I).

The following compounds are prepared in an analogous manner to the process described in Example 138:

### Examples

### 144 N-{(3S,4R,5R)-4-[4-((S)-4-Methoxy-3-methyl-butoxy)-phenyl]-5-[4-(3-methoxy-propyl)-3,4-dihydro-2H-benzo[1,4]oxazin-6-ylmethoxy]-piperidin-3-ylmethyl}-acetamide

using toluene-4-sulfonic acid (S)-4-methoxy-3-methyl-butyl ester

The starting materials are prepared as follows:

### a) Toluene-4-sulfonic Acid (S)-4-methoxy-3-methyl-butyl Ester

The title compound is prepared from (S)-4-methoxy-3-methyl-butan-1-ol in analogy to method H and identified from the residue on the basis of the Rf by flash chromatography (SiO₂ 60F).

### b) (S)-4-Methoxy-3-methyl-butan-1-ol

A solution of 1 mmol of (S)-4-methoxy-3-methyl-butyronitrile in 10 ml of methanol and 10 ml of a 2M aqueous sodium hydroxide solution is stirred for 16 hours at 80°. The methanol is mostly evaporated and the remaining aqueous phase is acidified to pH 2 with 2M HCl solution and extracted with ethylacetate (3×50 ml). The combined organic phases are washed with brine, dried with sodium sulfate and evaporated. The title compound is identified from the residue on the basis of the Rf by flash chromatography (SiO₂ 60F).

### c) (S)-4-Methoxy-3-methyl-butyronitrile

A mixture of 1 mmol of methanesulfonic acid (R)-3-methoxy-2-methyl-propyl ester and 5 mmol of sudium cyanide in 5 ml of dimethylsulfoxide is stirred for 16 hours at 60° C., then cooled to room temperature and 10 ml of water are added. The reaction mixture is extracted with tert-butyl methyl ether (3×50 ml), dried with sodium sulfate and evaporated. The title compound is identified from the residue on the basis of the Rf by flash chromatography (SiO₂ 60F).

### d) Methanesulfonic Acid (R)-3-methoxy-2-methyl-propyl Ester

To a solution of 1 mmol of (S)-3-methoxy-2-methyl-propan-1-ol [913969-31-0] and 5 mmol of triethylamine in 10 ml of dichloromethane are added dropwise 2 mmol of methanesulfonyl chlorid at 0° C. The reaction mixture is stirred for 3 hours at 0° C., then, the mixture is washed with water and 1 M aqueous citric acid solution, dried with sodium sulfate and evaporated. The title compound is obtained as a colourless oil from the residue by flash chromatography (SiO₂ 60F) and used crude in the next step. Rf=0.57 (diethyl ether).

### 149 N-{(3S,4R,5R)-4-[4-((R)-4-Methoxy-pentyloxy)-phenyl]-5-[4-(3-methoxy-propyl)-3,4-dihydro-2H-benzo[1,4]oxazin-6-ylmethoxy]-piperidin-3-ylmethyl}-acetamide

using toluene-4-sulfonic acid (R)-4-methoxy-pentyl ester

The starting material is prepared as follows:

### a) Toluene-4-sulfonic Acid (R)-4-methoxy-pentyl Ester

The title compound is obtained from (R)-3-methoxy-butan-1-ol [119784-98-4] in analogy to the process described in Example 144a, b, c and d and identified from the residue on the basis of the Rf by flash chromatography (SiO₂ 60F).

### Example 139

### 3-{(3S,4S,5R)-4-[4-(4-Methoxy-butoxy)-phenyl]-5-[4-(3-methoxy-propyl)-3,4-dihydro-2H-benzo[1,4]oxazin-6-ylmethoxy]-piperidin-3-yloxy}-propan-1-ol

The title compound is prepared from (3S,4S,5R)-3-(3-hydroxy-propoxy)-4-[4-(4-methoxy-butoxy)-phenyl]-5-[4-(3-methoxy-propyl)-3,4-dihydro-2H-benzo[1,4]oxazin-6-ylmethoxy]-piperidine-1-carboxylic acid benzyl ester in analogy to method B.

### a) (3S,4S,5R)-3-(3-Hyd roxy-propoxy)-4-[4-(4-methoxy-butoxy)-phenyl]-5-[4-(3-methoxy-propyl)-3,4-dihydro-2H-benzo[1,4]oxazin-6-ylmethoxy]-piperidine-1-carboxylic Acid Benzyl Ester

The title compound is obtained as a colourless oil from (3R,4S,5S)-4-[4-(4-methoxy-butoxy)-phenyl]-3-[4-(3-methoxy-propyl)-3,4-dihydro-2H-benzo[1,4]oxazin-6-ylmethoxy]-5-(3-triisopropylsilanyloxy-propoxy)-piperidine-1-carboxylic acid benzyl ester in analogy to method J. Rf=0.06 (EtOAc-heptane 2:1); Rt=5.01 (Gradient I).

### b) (3R,4S,5S)-4-[4-(4-Methoxy-butoxy)-phenyl]-3-[4-(3-methoxy-propyl)-3,4-dihydro-2H-benzo[1,4]oxazin-6-ylmethoxy]-5-(3-triisopropylsilanyloxy-propoxy)-piperidine-1-carboxylic Acid Benzyl Ester

The title compound is obtained as a colourless oil from (3S,4S,5R)-3-hydroxy-4-[4-(4-methoxy-butoxy)-phenyl]-5-[4-(3-methoxy-propyl)-3,4-dihydro-2H-benzo[1,4]oxazin-6-ylmethoxy]-piperidine-1-carboxylic acid benzyl ester (Example 78a) in analogy to the process described in Example 33a. Rf=0.25 (EtOAc-heptane 1:1); Rt=8.04 (Gradient 111).

1-11. (canceled)

12. A compound of the general formula 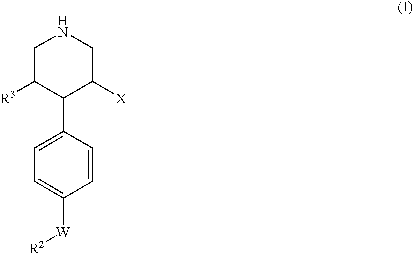
in which
(A) R¹ is heterocyclyl, in particular azepanyl, benzo[1,3]dioxolyl, benzofuranyl, benzoimidazolyl, 4H-benzo[1,4]oxazinyl, benzooxazolyl, 4H-benzo[1,4]thiazinyl, quinolinyl, chromenyl, dihydro-benzo[e][1,4]diazepinyl, dihydrobenzofuranyl, 3,4-dihydro-2H-benzo[1,4]oxazinyl, dihydro-3H-benzo[1,4]oxazinyl, dihydrobenzo[d][1,3]oxazinyl, 3,4-dihydro-2H-benzo[1,4]thiazinyl, dihydro-2H-1λ6-benzo[1,4]thiazinyl, dihydro-1H-quinazolinyl, 1a,7b-dihydro-1H-cyclopropa[c]chromenyl, dihydroimidazolyl, 1,3-dihydroindolyl, 2,3-dihydroindolyl, dihydro-1H-pyrido[2,3-b][1,4]oxazinyl, indazolyl, indolyl, 3H-isobenzofuranyl, [1,5]naphthyridyl, oxazolyl, phthalazinyl, piperidinyl, pyrazolyl, 1H-pyrido[2,3-b][1,4]oxazinyl, pyridyl, 1H-pyrrolizinyl, 1H-pyrrolo[2,3-b]pyridyl, pyrrolyl, tetrahydrobenzo[e][1,4]diazepinyl, 3H-thieno[2,3-d]pyrimidinyl, tetrahydro-quinoxalinyl, 1,1a,2,7b-tetrahydrocyclopropa[c]chromenyl, tetrahydropyranyl or triazinyl, substituted by oxo or oxide or
by 1-4-acetamidinyl-C₁₋₈alkyl, acyl-C₁₋₈alkoxy-C₁₋₈alkyl, (N-acyl)-C₁₋₈alkoxy-C₁₋₈alkylamino, C₁₋₈alkoxy, C₁₋₈alkoxy-C₁₋₈alkoxy, C₁₋₈alkoxy-C₁₋₈alkoxy-C₁₋₈alkyl, C₁₋₈alkoxy-C₁₋₈alkyl, (N—C₁₋₈alkoxy)-C₁₋₈alkylaminocarbonyl-C₁₋₈alkoxy, (N—C₁₋₈alkoxy)-C₁₋₈alkylaminocarbonyl-C₁₋₈alkyl, C₁₋₈alkoxy-C₁₋₈alkyl-carbamoyl, C₁₋₈alkoxy-C₁₋₈alkylcarbonyl, C₁₋₈alkoxy-C₁₋₈alkylcarbonylamino, 1-C₁₋₈-alkoxy-C₁₋₈alkylimidazol-2-yl, 2-C₁₋₈alkoxy-C₁₋₈alkyl-4-oxoimidazol-1-yl, 1-C₁₋₈alkoxy-C₁₋₈-alkyltetrazol-5-yl, 5-C₁₋₈alkoxy-C₁₋₈-alkyltetrazol-1-yl, 6-alkoxy-aminocarbonyl-C₁₋₈, C₁₋₈alkoxyaminocarbonyl-C₁₋₈alkyl, C₁₋₈, C₁₋₈alkoxycarbonyl-C₁₋₈alkoxy, C₁₋₈alkoxycarbonyl-C₁₋₈, C₁₋₈alkoxycarbonylamino-C₁₋₈alkoxy, C₁₋₈alkoxycarbonylamino-C₁₋₈alkyl, C₁₋₈alkyl, (N—C₁₋₈alkyl)-C₁₋₈alkoxy-C₁₋₈alkylcarbamoyl, (N—C₁₋₈alkyl)-C₁₋₁₈alkoxy-C₁₋₈alkylcarbonylamino, (N—C₁₋₈alkyl)-C₁₋₈alkoxycarbonylamino, (N—C₁₋₈alkyl)-C₀₋₈alkylcarbonylamino-C₁₋₈alkoxy, (N—C₁₋₈alkyl)-C₀₋₈alkylcarbonylamino-C₁₋₈alkyl, (N—C₁₋₈alkyl)-C₁₋₈alkylsulfonylamino-C₁₋₈alkoxy, (N—C₁₋₈alkyl)-C₁₋₈alkylsulfonylamino-C₁₋₈alkyl, C₁₋₈alkylamidinyl, C₁₋₈alkylaminocarbonyl-C₁₋₈, di-C₁₋₈alkylaminocarbonyl-C₁₋₈alkoxy, C₁₋₈alkylaminocarbonyl-C₁₋₈alkoxy-C₁₋₈alkyl, C₁₋₈alkylaminocarbonyl-C₁₋₈alkyl, C₁₋₈alkylaminocarbonylamino-C₁₋₈alkoxy, C₁₋₈alkylaminocarbonylamino-C₁₋₈alkyl, di-C₁₋₈alkylaminocarbonyl-C₁₋₈ alkyl, C₁₋₈alkylamino-C₂₋₈alkoxy, di-C₁₋₈alkylamino-C₂₋₈alkoxy, C₁₋₈alkylamino-C₁₋₈alkyl, di-C₁₋₈alkylamino-C₁₋₈alkyl, C₁₋₈alkylcarbamoyl, di-C₁₋₈alkylcarbamoyl, C₀₋₈alkylcarbonylamino-C₁₋₈alkoxy, C₀₋₈alkylcarbonylamino, C₀₋₈alkylcarbonylamino-C₁₋₈alkyl, C₁₋₈alkylcarbonyloxy-C₁₋₈alkoxy, C₁₋₈alkylcarbonyloxy-C₀₋₈alkyl, C₁₋₈alkylsulfonyl, C₁₋₈alkylsulfonyl-C₁₋₈alkoxy, C₁₋₈alkylsulfonyl-C₁₋₈alkyl, C₁₋₈alkylsulfonylamino-C₁₋₈alkoxy, C₁₋₈alkylsulfonylamino-C₁₋₈alkyl, carbamoyl, carbamoyl-C₁₋₈alkoxy, carbamoyl-C₁₋₈alkyl, carboxy-C₁₋₈alkoxy, carboxy-C₁₋₈alkoxy-C₁₋₈alkyl, carboxy-C₁₋₈alkyl, cyano, cyano-C₁₋₈alkoxy, cyano-C₁₋₈alkyl, C₃₋₈cycloalkyl-C₁₋₈alkoxy, C₃₋₈cycloalkyl-C₁₋₈alkyl, C₃₋₈cycloalkylcarbonyl-amino-C₁₋₈alkoxy, C₃₋₈cycloalkylcarbonylamino-C₁₋₈alkyl, O,N-dimethylhydroxylamino-C₁₋₈alkyl, halogen, hydroxy-C₁₋₈ alkoxy-C₁₋₈alkoxy, hydroxy-C₁₋₈alkoxy-C₁₋₈alkyl, hydroxy-C₁₋₈alkyl, (N-hydroxy)-C₁₋₈alkylaminocarbonyl-C₁₋₈alkoxy, (N-hydroxy)-C₁₋₈alkylaminocarbonyl-C₁₋₈alkyl, (N-hydroxy)aminocarbonyl-C₁₋₈alkoxy, (N-hydroxy)aminocarbonyl-C₁₋₈alkyl, 2-oxooxazolidinyl-C₁₋₈alkoxy, 2-oxooxazolidinyl-C₁₋₈alkyl, O-methyloximyl-C₁₋₈ alkyl, polyhalo-C₁₋₈alkoxy or polyhalo-C₁₋₈alkyl; or by 3-acetamidomethylpyrrolidinyl, 3-C₁₋₈alkoxy-C₁₋₈alkyl-pyrrolidinyl, 3,4-dihydroxypyrrolidinyl, 2,6-dimethylmorpholinyl, 3,5-dimethylmorpholinyl, dioxanyl, dioxolanyl, 4,4-dioxothiomorpholinyl, dithianyl, dithiolanyl, 2-hydroxymethylpyrrolidinyl, 4-hydroxypiperidinyl, 3-hydroxypyrrolidinyl, imidazolyl-alkoxy, imidazolylalkyl, 2-methylimidazolylalkoxy, 2-methylimidazolylalkyl, 3-methyl-[1,2,4]-oxadiazol-5-ylalkoxy, 5-methyl-[1,2,4]-oxadiazol-3-ylalkoxy, 3-methyl-[1,2,4]-oxadiazol-5-ylalkyl, 5-methyl-[1,2,4]-oxadiazol-3-ylalkyl, 4-methylpiperazinyl, 5-methyltetrazol-1-ylalkoxy, 5-methyltetrazol-1-ylalkyl, morpholinyl, [1,2,4]-oxadiazol-5-ylalkoxy, [1,2,4]-oxadiazol-5-ylalkyl, oxazol-4-ylalkoxy, oxazol-4-ylalkyl, 2-oxo-[1,3]oxazinyl, 2-oxooxazolidinyl, 2-oxoimidazolidinyl, 2-oxopyrrolidinyl, 4-oxo-piperidinyl, 2-oxopyrrolidinylalkoxy, 2-oxopyrrolidinylalkyl, 2-oxotetrahydropyrimidinyl 4-oxothiomorpholinyl, piperazinyl, piperidinyl, pyrrolidinyl, pyrrolyl, [1,2,4]-triazol-1-yl-alkoxy, [1,2,4]-triazol-4-ylalkoxy, [1,2,4]-triazol-1-ylalkyl, [1,2,4]-triazol-4-ylalkyl, tetrazol-1-ylalkoxy, tetrazol-2-ylalkoxy, tetrazol-5-ylalkoxy, tetrazol-1-ylalkyl, tetrazol-2-ylalkyl, tetrazol-5-ylalkyl, thiazol-4-ylalkoxy, thiazol-4-ylalkyl or thiomorpholinyl;
R² a) is absent when W is cyano; or
b) is C₁₋₈alkyl, C₂₋₈alkenyl, C₂₋₈alkynyl, C₁₋₈alkoxy-C₁₋₈alkyl, C₁₋₈alkoxy-C₃₋₈cycloalkyl-C₁₋₈-alkyl, C₁₋₈alkylsulfanyl-C₁₋₈alkyl, C₁₋₈alkylsulfonyl-C₁₋₈alkyl when W is —O— or —S—;
R³ a) is halogen- and/or hydroxy-substituted C₁₋₈alkoxy, halogen- and/or hydroxy-substituted C₁₋₈alkoxy-C₁₋₈alkoxy, branched C₁₋₈alkoxy-C₁₋₈alkoxy, optionally N-mono- or N,N-di-C₁₋₈-alkylated amino-C₁₋₈alkoxy, optionally N—C₁₋₈alkylated C₁₋₈alkoxy-C₁₋₈alkylamino-C₁₋₈alkoxy, optionally N-mono- or N,N-di-C₁₋₈alkylated amino-C₀₋₈-alkylcarbonyl-C₁₋₈alkoxy, hydroxy-C₀₋₈-alkylcarbonyl-C₀₋₈alkoxy, C₁₋₈alkoxy-C₀₋₈alkylcarbonyl-C₀₋₈alkoxy, C₁₋₈-alkylcarbonylamino-C₁₋₈alkoxy, cyano-C₁₋₈alkoxy, substituted C₃₋₈cycloalkyl-C₀₋₈alkoxy, heterocyclyl-C₀₋₈alkoxy, optionally N—C₁₋₈alkylated heterocyclyl-C₀₋₈alkylamino-C₀₋₈alkyl-carbonyl-C₀₋₈alkoxy, C₁₋₈alkyl-sulfonyl-C₁₋₈alkoxy, C₂₋₈alkynyloxy, heterocyclyl-C₂₋₈alkynyl-oxy, optionally N-mono- or N,N-di-C₁₋₈alkylated amino-C₂₋₈alkynyloxy, N-mono- or N,N-di-C₁₋₈alkylated aminocarbonyl-C₂₋₈alkynyloxy, heterocyclylcarbonyl-C₀₋₈alkoxy, optionally N-mono- or N,N-di-C₁₋₈alkylated amino-C₁₋₈alkyl, optionally N—C₁₋₈alkylated C₁₋₈alkoxy-C₁₋₈alkylamino-C₁₋₈alkyl, optionally N-mono- or N,N-di-C₁₋₈alkylated and optionally hydroxy-substituted amino-C₀₋₈alkyl-carbonyl-C₀₋₈alkyl, optionally N—C₁₋₈alkylated heterocyclyl-C₀₋₈alkylamino-C₀₋₈alkylcarbonyl-C₀₋₈alkyl, optionally halogen- or hydroxy-substituted C₁₋₈alkoxy-C₁₋₈alkyl, optionally halogen- or hydroxy-substituted hydroxy-C₁₋₈alkyl, optionally N—C₁₋₈alkylated hydroxy-C₁₋₈alkylamino-C₁₋₈alkyl, heterocyclylcarbonyl-C₀₋₈alkyl, heterocyclylcarbonyl-C₀₋₈alkylamino-C₁₋₈alkyl, heterocyclyl-C₁₋₈alkyl, C₁₋₈alkoxy-carbonylamino-C₁₋₈alkyl, optionally halogen-substituted heterocyclyl-C₀₋₈alkylcarbonyl-amino-C₁₋₈alkyl, optionally halogen-substituted C₃₋₈cycloalkyl-C₀₋₈alkylcarbonylamino-C₁₋₈alkyl or optionally halogen-substituted C₁₋₈alkylcarbonylamino-C₁₋₈alkyl; or additionally b) is hydroxy, unsubstituted C₁₋₈alkoxy, unsubstituted, unbranched C₁₋₈alkoxy-C₁₋₈-alkoxy or unsubstituted C₃₋₈cycloalkyl-C₀₋₈alkoxy if —W—R² is not C₁₋₈alkoxy;
R⁴ is acyl, C₁₋₈alkoxy-C₁₋₈alkyl, C₁₋₈alkyl, aryl-C₁₋₈alkyl, C₃₋₈cycloalkyl-C₀₋₈alkyl, or hydrogen;
R⁵ is C₁₋₈alkoxycarbonyl-C₁₋₈alkyl, C₁₋₈alkyl, carboxy-C₁₋₈alkyl or hydrogen;
R⁶ is acyl, C₂₋₈alkenyl, C₁₋₈alkyl, aryl-C₁₋₈alkyl or hydrogen;
X is Z, —O-Z or —S-Z, where the bond originating from an oxygen or sulfur atom leads to a saturated C atom of the group Z, or is a group —CHR⁶-Z, —CHOR⁴-Z, —O—CO-Z, —O—CO—R¹, —CO-Z, —C═NOR⁵-Z, —O—CHR⁶-Z, —O—CHR⁶—CO—NR⁴-Z, —O—CHR⁶—CO—NR⁴—R¹, or —O—CHR⁶—R¹;
W is —O—, —S— or cyano;
Z is C₁₋₈-Alk-R¹, C₂₋₈alkenylene-R¹, hydroxy-substituted −Alk-R¹, —O—R¹, —S—R¹, —O-Alk-R¹, —S-Alk-R¹, −Alk-O—R¹, −Alk-S—R¹ or −Alk-NR⁴—R¹, where Alk is C₁₋₈alkylene; and where
(a) X is —CH—R⁶-Z if Z is —O—R¹ or —S—R¹
(b) X is —CH—R⁶-Z if Z is —O-Alk-R¹ or —S-Alk-R¹; and
(c) Z is C₂₋₈alkenylene-R¹, −Alk-O—R¹, −Alk-S—R¹ or −Alk-NR⁴—R¹ if X is Z;
and a salt thereof, preferably a pharmaceutically acceptable salt thereof.

13. A compound according to claim 12, which corresponds to the general formula (IA) 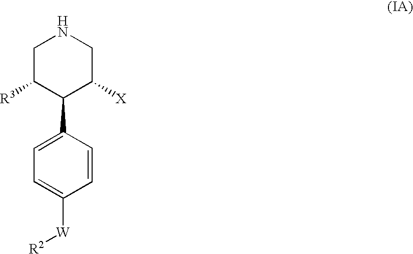
and a salt thereof, preferably a pharmaceutically acceptable salt thereof, where the meanings of the substituents R², R³, W and X are as indicated for compounds of the formula (I) according to claim 12.

14. A compound according to claim 12 or 13, wherein
X is —CHR⁶-Alk-R¹, −Alk-NR⁴—R¹, −Alk-O—R¹, −Alk-S—R₁, C₂₋₈-Alkenylen-R¹, —CH(OR⁴)-Alk-R¹, —CHR⁶-Alk-R¹, —CHR⁶—O—R¹, —CHR⁶—O-Alk-R¹, —CHR⁶—S—R¹, —CHR⁶—S-Alk-R¹, —CO-Alk-R¹, —C(═NOR⁵)-Alk-R¹, —O-Alk-NR⁴—R¹, —O-Alk-R¹, —O-Alk-O—R¹, —O—CO—R¹, —O—CO-Alk-R¹, —O—CHR⁶—R¹, —O—CHR⁶-Alk-R¹, —O—CHR⁶—CO—NR⁴—R¹ or —O—CHR⁶—CO—NR⁴-Alk-R¹, where Alk is C₁₋₈alkylene.

15. A compound according to claim 12 or 13, wherein
R³ a) is halogen- and/or hydroxy-substituted C₁₋₈alkoxy, halogen- and/or hydroxy-substituted C₁₋₈alkoxy-C₁₋₈alkoxy, branched C₁₋₈alkoxy-C₁₋₈alkoxy, optionally N-mono- or N,N-di-C₁₋₈alkylated amino-C₁₋₈alkoxy, optionally N-mono- or N,N-di-C₁₋₈alkylated amino-C₀₋₈alkylcarbonyl-C₁₋₈alkoxy, substituted C₃₋₈cycloalkyl-C₀₋₈alkoxy, optionally C₁₋₈alkoxy or hydroxy-substituted heterocyclyl-C₀₋₈alkoxy, heterocyclylcarbonyl-C₀₋₈alkoxy, heterocyclyl-carbonyl-C₀₋₈alkyl, optionally halogen-substituted heterocyclyl-C₀₋₈alkyl-carbonylamino-C₁₋₈alkyl, optionally halogen-substituted C₃₋₈cycloalkyl-C₀₋₈alkylcarbonyl-amino-C₁₋₈alkyl or optionally halogen-substituted C₁₋₈alkylcarbonylamino-C₁₋₈alkyl; or additionally b) hydroxy, unsubstituted C₁₋₈alkoxy, unsubstituted, unbranched C₁₋₈alkoxy-C₁₋₈alkoxy or unsubstituted C₃₋₈cycloalkyl-C₀₋₈alkoxy if-W—R² is not C₁₋₈alkoxy.

16. A compound according to claim 12 or 13, wherein
R² is C₁₋₈alkyl, C₂₋₈alkenyl, C₁₋₈alkoxy-C₁₋₈alkyl, C₁₋₈alkoxy-C₃₋₈cycloalkyl-C₁₋₈alkyl, C₃₋₈cycloalkyl-C₀₋₈alkoxy-C₁₋₈alkyl-, C₁₋₈alkylsulfanyl-C₁₋₈alkyl, C₁₋₈alkylsulfonyl-C₁₋₈alkyl;
R³ a) hydroxy-substituted C₁₋₈alkoxy, hydroxy-substituted C₁₋₈alkoxy-C₁₋₈alkoxy, branched C₁₋₈alkoxy-C₁₋₈alkoxy or C₁₋₈alkylcarbonylamino-C₁₋₈alkyl; or additionally
b) hydroxy, unsubstituted C₁₋₈alkoxy or unsubstituted, unbranched C₁₋₈alkoxy-C₁₋₈alkoxy if R² is not C₁₋₈alkyl;
and
W is —O—.

17. A compound according to claim 12 or 13, wherein
R¹ is substituted chromenyl or 3,4-dihydro-2H-benzo[1,4]oxazinyl;
R² is C₁₋₈alkyl, C₂₋₈alkenyl, C₁₋₈alkoxy-C₁₋₈alkyl, C₁₋₈alkoxy-C₃₋₈cycloalkyl-C₁₋₈alkyl, C₃₋₈cycloalkyl-C₀₋₈alkoxy-C₁₋₈alkyl, C₁₋₈alkylsulfanyl-C₁₋₈alkyl;
R³ a) hydroxy-substituted C₁₋₈alkoxy, hydroxy-substituted C₁₋₈alkoxy-C₁₋₈alkoxy, branched C₁₋₈alkoxy-C₁₋₈alkoxy or C₁₋₈alkylcarbonylamino-C₁₋₈alkyl; or additionally
b) hydroxy, unsubstituted C₁₋₈alkoxy or unsubstituted, unbranched C₁₋₈alkoxy-C₁₋₈alkoxy if R² is not C₁₋₈alkyl;
R⁶ is C₁₋₈alkyl or hydrogen;
X is —CHR⁶-Alk-R¹ or —O-Alk-R¹, where Alk is C₁₋₈alkylene; and
W is —O—.

18. A method for preventing, for delaying the progression of or for treating high blood pressure, heart failure, glaucoma, myocardial infarction, renal failure, restenoses or stroke, where a therapeutically effective amount of a compound of the general formula (I) or of its pharmaceutically acceptable salt, according to claim 12 or 13 is used.

19. A pharmaceutical product comprising a compound of the general formula (I) or its pharmaceutically acceptable salt, according to claim 12 or 13, and conventional excipients.

20. A pharmaceutical combination in the form of a product or of a kit composed of individual components consisting a) of a compound of the general formula (I) or of its pharmaceutically acceptable salt, according to claim 12 or 13, and b) at least one pharmaceutical form as active ingredient having a cardiovascular effect.
